(19) United States
(12) Patent Application Publication
(10) Pub. No.: US 2026/0317439 A1
(43) Pub. Date: Oct. 15, 2026

(54) MULTI-SPECIFIC POLYPEPTIDE COMPLEXES

(71) Applicants: SHANGHAI KAIJIN BIOTECHNOLOGY LTD, Shanghai (CN); CHIMAGEN BIOSCIENCES, LTD, Shanghai (CN)

(72) Inventors: Zhenhao ZHOU, Shanghai (CN); Jie ZHANG, Shanghai (CN); Gong WANG, Shanghai (CN)

(21) Appl. No.: 18/998,128

(22) PCT Filed: Feb. 7, 2023
(86) PCT No.: PCT/CN2023/074836

§ 371 Date: Jan. 24, 2025

(30) Foreign Application Priority Data
Aug. 5, 2022 (WO) PCT/CN2022/110697

(51) Int. Cl. C07K 16/28; C07K 14/47; C07K 14/705

(52) U.S. Cl. CPC C07K 16/2887; C07K 14/4702; C07K 14/4716; C07K 14/7051; C07K 16/2809; C07K 2317/31; C07K 2317/522; C07K 2317/624; C07K 2319/00

(57) ABSTRACT

The present disclosure provides novel covalent multi-specific antibodies and uses thereof.

### FIELD OF THE INVENTION

The present invention relates to novel covalent multi-specific antibodies and uses thereof.

### BACKGROUND

A bispecific antibody is an artificial antibody capable of binding to at least two antigens. By simultaneous engagement of at least two targets of interest, the bi-specific antibody can deliver benefits superior to conventional monospecific antibodies via novel and unique mechanisms. For example, Blinatumomab (CD3×CD19, Amgen) that targets CD3 and CD19 can efficiently engage T cells in the killing of CD19-expressing tumor cells via its CD3-recognizing Fv and showed superior efficacy over conventional antibodies in treating ALL (acute lymphoid leukemia) etc. Blinatumomab was approved to launch for ALL treatment by FDA in 2014.

A number of bi-specific antibody technology platforms have been developed, to name a few among them: BiTE Bi-specific T-cell Engaging (Micromet, acquired by Amgen in 2012), CrossMab (Roche), DVD-Ig (Abbvie), TandAb (Affimed), and DART (Dual Antigen Re-Targeting, Macrogenics).

However, despite of the advantages conferred by bi-specific or multi-specific antibodies, they also introduce challenges, for example, in manufacture. Mispairing can happen between heavy chains and/or between heavy chain and light chain. How to efficiently and effectively remove the mispaired by-products has been an insurmountable challenge for bispecific or multi-specific antibodies. Therefore, there exists great needs to develop novel constructs that can provide for both good binding affinity to targets of interest but are also convenient for manufacture and downstream purification.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides, among others, novel fusion polypeptides, nucleotide sequences encoding such, and the uses thereof.

In one aspect, the present disclosure provides a fusion polypeptide comprising from C terminus to N terminus: a) a first target-binding fragment A1; b) a polypeptide linker; c) a second target-binding fragment B2, wherein the polypeptide linker has a length that is sufficiently short to minimize potential intramolecular interaction between A1 and B2. In some embodiments, the A1 is capable of pairing with a first pairing fragment B1, to form a first target binding domain; the B2 is capable of pairing with a second pairing fragment A2, to form a second target binding domain. In such embodiments, the A1 is configured to exhibit less binding to the B2 relative to the B1, and the B2 is configured to exhibit less binding to the A1 relative to the A2.

In another aspect, the present disclosure provides a polypeptide complex comprising: a) the fusion polypeptide provided herein; b) a second polypeptide comprising the first pairing fragment B1; c) a third polypeptide comprising the second target binding fragment B2; and d) a fourth polypeptide and the fifth polypeptide each comprising the second pairing fragment A2, wherein the A1 in the fusion polypeptide pairs with the B1 in the second polypeptide to form a first target binding domain; the B2 in the fusion polypeptide pairs with the A2 in the fourth polypeptide to form a second target binding domain; and the A2 in the fifth polypeptide pairs with the B2 in the third polypeptide to form another second target binding domain.

In some embodiments, at least one of the pair of B1 and A1 and the pair of B2 and A2 contains at least one configuration capable of discouraging mispairing between B1 and A2 and/or between B2 and A1.

In some embodiments, the A1 comprises a first antibody variable region VA1 selected from VH1 or VL1. In such embodiments, the B1 comprises a first pairing antibody variable region VB1 capable of pairing with VA1 to form the first target-binding domain, wherein the VB1 is selected from VH1 or VL1.

In some embodiments, the B2 comprises a second antibody variable region VB2 selected from VH2 or VL2. In such embodiments, the A2 comprises a second pairing antibody variable region VA2 capable of pairing with VB2 to form the second target-binding domain, wherein the VA2 is selected from VH2 or VL2.

In some embodiments, the VB1 comprises VH1, the VA1 comprises VL1, the VB2 comprises VH2, and the VA2 comprises VL2.

In some embodiments, the VB1 comprises VH1, the VA1 comprises VL1, the VB2 comprises VL2, and the VA2 comprises VH2. In some other embodiments, the VB1 comprises VL1, the VA1 comprises VH1, the VB2 comprises VH2, and the VA2 comprises VL2.

In some embodiments, the A1 further comprises a first scaffold region SRₐ operably linked to the VA1, and the B2 further comprises a second scaffold region SR_{b} operably linked to the VB2, wherein the SRₐ and the SR_{b} are configured such that pairing between the SRₐ and the SR_{b} is discouraged.

In some embodiments, the B1 further comprises a first pairing scaffold region PSRₐ that is operably linked to the VB1 and is capable of binding to the SRₐ, and wherein the A2 further comprises a second pairing scaffold region PSR_{b} that is operably linked to the VA2 and is capable of binding to the SR_{b}.

In some embodiments, the pair of SRₐ/PSRₐ or the pair of SR_{b}/PSR_{b} is/are selected from the group consisting of: a) a pair of heavy chain constant region 1 (CH1) and light chain constant region (CL); b) a pair of T cell receptor (TCR) constant region alpha (Cₐₗₚₕₐ) and TCR constant region beta (C_{beta}); c) a pair of TCR constant region gamma (C_{gamma}) and TCR constant region delta (C_{delta}); d) a pair of ligand-binding domain of a receptor and the ligand; and e) a pair of PRD (proline rich domain) and SH3 domain; f) a pair of obscurin and titin.

In some embodiments, the pair of the SRₐ and the PSRₐ is distinct from the pair of the SR_{b} and the PSR_{b}. In some of these embodiments, the pair of the SRₐ and the PSRₐ is a pair of CH1 and CL, and the pair of the SR_{b} and the PSR_{b} is a pair of i) Cₐₗₚₕₐ and C_{beta}, ii) C_{gamma} and C_{delta}, iii) ligand-binding domain of a receptor and the ligand, iv) PRD (proline rich domain) and SH3 domain, or v) obscurin and titin. In some other of these embodiments, the pair of the SR_{b} and the PSR_{b} is a pair of CH1 and CL, and the pair of the SRₐ and the PSRₐ is a pair of i) Cₐₗₚₕₐ and C_{beta}, ii) C_{gamma} and C_{delta}, iii) ligand-binding domain of a receptor and the ligand, iv) PRD (proline rich domain) and SH3 domain, or v) obscurin and titin.

In some embodiments, both the pair of SRₐ/PSRₐ and the pair of SR_{b}/PSR_{b} are the same, and the pair of SRₐ/PSRₐ and/or the pair of SR_{b}/PSR_{b} are configured such that mispairing between SRₐ/PSR_{b} or between SR_{b}/PSRₐ is discouraged. In some of these embodiments, the pair of SRₐ/PSRₐ comprises a CH1 domain CH1a and a CL domain CLa, and the pair of SR_{b}/PSR_{b} comprises a CH1 domain CH1b and a CL domain CLb.

In some embodiments, the VB1 comprises VH1, the VA1 comprises VL1, the VB2 comprises VH2, and the VA2 comprises VL2. In some of these embodiments, one pair of the CH1 domain and the CL domain is crossed. For example, the PSRₐ is CL domain CLa, the SRₐ is CH1 domain CH1a, the SR_{b} is CH1 domain CH1b, the PSR_{b} is CL domain CLb. Alternatively, the PSRₐ is CH1 domain CH1a, the SRₐ is CL domain CLa, the SR_{b} is CL domain CLb, the PSR_{b} is CH1 domain CH1b.

In some embodiments, the PSRₐ is CH1 domain CH1a, the SRₐ is CL domain CLa, the SR_{b} is CH1 domain CH1b, the PSR_{b} is CL domain CLb. In some of these embodiments, one pair of the VH domain and the VL domain is crossed. For example, the VB1 comprises VH1, the VA1 comprises VL1, the VB2 comprises VL2, and the VA2 comprises VH2. Alternatively, the VB1 comprises VL1, the VA1 comprises VH1, the VB2 comprises VH2, and the VA2 comprises VL2.

In certain embodiments, the VB1 comprises VH1, the VA1 comprises VL1, the VB2 comprises VH2, and the VA2 comprises VL2, the PSRₐ is CH1 domain CH1a, the SRₐ is CL domain CLa, the SR_{b} is CH1 domain CH1b, the PSR_{b} is CL domain CLb, and wherein: a) the fusion polypeptide comprises an amino acid sequence of Formula (I): VH2-CH1b-Linker-VL1-CLa; b) the second polypeptide comprises an amino acid sequence of Formula (II): VH1-CH1a; c) the third polypeptide comprises an amino acid sequence of Formula (III): VH2-CH1b; d) the fourth and fifth polypeptides each comprising an amino acid sequence of Formula (IV): VL2-CLb; and wherein the pair of CH1b/CLb and/or the pair of CH1a/CLa are configured such that mispairing between CH1b and CLa and/or between CH1a and CLb is discouraged.

In some embodiments, at least one of the pair of CH1b/CLb and the pair of CH1a/CLa have one or more characteristics shown below: 1) having at least one non-natural disulfide bond that discourages mispairing between CH1b and CLa and/or between CH1a and CLb; 2) having at least one or more introduced charged amino acid residues that discourages mispairing between CH1b and CLa and/or between CH1a and CLb; or 3) having one or more introduced amino acid mutations that form an orthogonal CH1-CL interface that discourages mispairing between CH1b and CLa or between CH1a and CLb. These characteristics are useful for discouraging mispairing between CH1b and CLa and/or between CH1a and CLb.

In some embodiments, at least one of the pair of CH1b/CLb and the pair of CH1a/CLa has at least one non-natural disulfide bond that discourages mispairing between CH1b and CLa and/or between CH1a and CLb. In some of these embodiments, a first CH1/CL pair and a second CH1/CL pair are selected from CH1b/CLb and CH1a/CLa, and wherein the first CH1/CL pair is associated by a first disulfide bond that is non-natural, and optionally lacks a natural disulfide bond or has a disrupted natural disulfide bond. In some embodiments, the second CH1/CL pair is associated by a second disulfide bond which is formed at a position different from the first disulfide bond, and optionally the second disulfide bond is a natural disulfide bond. In some embodiments, the first disulfide bond is formed by two cysteine residues introduced at a set of heavy chain-light chain EU positions selected from the group consisting of: a) heavy chain EU position 126—light chain EU position 121, b) heavy chain EU position 173—light chain EU position 160, and c) heavy chain EU position 128—light chain EU position 118. In some embodiments, the natural disulfide bond is between heavy chain EU position 220 and light chain EU position 214. In certain embodiments, the first CH1/CL pair comprises a CH1 comprising substitution at EU position 126 for a cysteine residue, and substitution at EU position 220 for a non-cysteine residue; and a CL comprising substitution at EU position 121 for a cysteine residue, and substitution at EU position 214 for a non-cysteine residue.

In some embodiments, at least one of the pair of CH1b/CLb and the pair of CH1a/CLa has at least one or more introduced charged amino acid residues that discourages mispairing between CH1b and CLa and/or between CH1a and CLb. In some embodiments, a first CH1/CL pair and a second CH1/CL pair are selected from CH1b/CLb and CH1a/CLa, and wherein the first CH1/CL pair contains at least one substitution of a non-charged residue to a charged residue, and/or at least one substitution of a charged residue to an oppositely charged residue, such that the first CH1/CL pair contains a first pair of oppositely charged residues that favors pairing of the first CH1/CL pair. In some embodiments, the second CH1/CL pair contains at least one substitution of a non-charged residue to a charged residue, and/or at least one substitution of a charged residue to an oppositely charged residue, such that the second CH1/CL pair contains a second pair of oppositely charged residues that favors pairing of the second CH1/CL pair, and optionally the first pair of oppositely charged residues and the second pair of oppositely charged residues discourages pairing of CH1a with CLb or CH1b and CLa. In some embodiments, the first pair of oppositely charged residues and/or the second pair of oppositely charged residues are configured such that CH1a and CLb have both positive or both negative charged residues, and/or CH1b and CLa have both positive or both negative charged residues. In certain embodiments, wherein the first pair of oppositely charged residues and/or the second pair of oppositely charged residues is introduced at a set of heavy chain-light chain EU positions selected from the group consisting of: a) heavy chain EU position 183: light chain EU position 176, b) heavy chain EU position 183: light chain EU position 133, c) heavy chain EU position 147: light chain EU position 176, d) heavy chain EU position 141: light chain EU position 116, e) heavy chain EU position 126: light chain EU position 121, and f) heavy chain EU position 218: light chain EU position 122. In certain embodiments, the pair of oppositely charged residues comprises a positive-charged amino acid residue and a negative-charged amino acid residue, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

In some embodiments, at least one of the pair of CH1b/CLb and the pair of CH1a/CLa has one or more introduced amino acid mutations that form an orthogonal CH1-CL interface that discourages mispairing between CH1b and CLa or between CH1a and CLb. In some embodiments, a first CH1/CL pair and a second CH1/CL pair are selected respectively from CH1b/CLb and CH1a/CLa, and wherein the first CH1/CL pair comprises one or more introduced amino acid mutations that form an orthogonal CH1-CL interface. In some embodiments, the orthogonal CH1-CL interface introduced at a set of heavy chain-light chain EU positions including heavy chain EU positions H168A, F170G, and light chain EU positions L135Y, S176W. In some embodiments, the first CH1/CL pair comprises one or more introduced amino acid mutations that form orthogonal Fab designs at a set of heavy chain-light chain EU positions selected from the group consisting of: a) substitutions at heavy chain EU positions A141I, F170S, S181M, S183A, and V185A, and substitutions at light chain EU positions F116A, A235V, S174A, S176F, and T178V.

In some embodiments, the pair of VH1/VL1 and/or the pair of VH2/VL2 are configured such that mispairing between VH1 and VL2 and/or between VH2 and VL1 is discouraged. In some of these embodiments, a first VH/VL pair and a second VH/VL pair are selected from VH1/VL1 and VH2/VL2, and wherein the first VH/VL pair has at least one substitution of a non-charged residue to a charged residue, and/or at least one substitution of a charged residue to an oppositely charged residue, such that the first VH/VL contains a third pair of oppositely charged residues that favors pairing of first VH/VL pair. In some embodiments, the second VH/VL pair has at least one substitution of a non-charged residue to a charged residue, and/or at least one substitution of a charged residue to an oppositely charged residue, such that the second VH/VL contains a fourth pair of oppositely charged residues that favors pairing of second VH/VL pair, and optionally the third pair of oppositely charged residues and the fourth pair of oppositely charged residues discourages pairing of VH1 with VL2 or VH2 with VL1. In some of these embodiments, the third pair of oppositely charged residues and the fourth pair of oppositely charged residues are configured such that VH1 and VL2 have both positive or both negative charged residues, and/or VH2 and VL1 have both positive or both negative charged residues. In some embodiments, the third pair of oppositely charged residues and/or the fourth pair of oppositely charged residues is introduced at a set of heavy chain-light chain EU positions selected from the group consisting of: a) heavy chain EU position 39: light chain EU position 38; b) heavy chain EU position 105: light chain EU position 43, and c) heavy chain EU position 62: light chain EU position 1, or any combination thereof. In some embodiments, the pair of oppositely charged residues comprises a positive-charged amino acid residue and a negative-charged amino acid residue, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

In some embodiments, the second polypeptide and the third polypeptide further comprise an operably linked first dimerization domain and an operably linked second dimerization domain, respectively, that are associated to form a dimer; optionally, the first dimerization domain comprises a first Fc region, and/or the second dimerization domain comprises a second Fc region. In some embodiments, the first Fc region and/or the second Fc region of the polypeptide complex is derived from IgG1, IgG2, IgG3 or IgG4. In some embodiments, the first Fc region and the second Fc region of the polypeptide complex have different amino acid sequences and have at least one configuration that facilitates heterodimerization of the first Fc region and the second Fc region.

In some embodiments, the first Fc region of the polypeptide complex comprises a first Fc mutation, and/or the second Fc region of the polypeptide complex comprises a second Fc mutation, wherein: a) the first Fc mutations comprises T366W or S354C, and the second Fc mutation comprises Y349C, T366S, L368A, or Y407V; b) the first Fe mutation comprises D399K or E356K, and the second Fc mutation comprises K392D, or K409D; c) the first Fc mutation comprises E356K, E357K, or D399K, and the second Fc mutation comprises K370E, K409D, or K439E; d) the first Fc mutation comprises S364H, or F405A, and the second Fc mutation comprises Y349T, or T394F; e) the first Fc mutation comprises S364H, or T394F, and the second Fc mutation comprises Y394T, or F405A; f) the first Fc mutation comprises K370D, or K409D, and the second Fc mutation comprises E357K, or D399K; or g) the first Fc mutation comprises L351D, or L368E, and the second Fc mutation comprises L351K, or T366K, wherein numbering is according to the EU index.

In another aspect, at least one of the first target binding domain and the second target binding domain is chimeric, humanized, or fully human.

In some embodiments, the first target binding domain and the second target binding domain bind to different targets. In some embodiments, at least one of the first target binding domain and the second target binding domain binds to a tumor-associated antigen, or an immune-related target. In some embodiments, one of the first target binding domain and the second target binding domain binds to a tumor-associated antigen, and the other binds to an immune-related target. In some embodiments, at least one of the first target binding domain and the second target binding domain binds to a disease-associated antigen or an immune related target, optionally, the disease-associated antigen is a tumor-associated antigen, an antigen associated with an autoimmune diseases or an inflammatory disease, or an antigen associated with an eye disorder, an antigen associated with central nervous system disease, an antigen associated with an infectious disease or an antigen associated with a coagulation disease.

Another aspect of the present disclosure provides a nucleic acid comprising a nucleotide sequence encoding the fusion polypeptide or the polypeptide complex as described herein.

In yet another aspect, the present disclosure provides a vector comprising the nucleic acid described herein.

In yet another aspect, the present disclosure provides a host cell comprising the nucleic acid and the vector described herein.

In yet another aspect, the present disclosure provides a pharmaceutical composition comprising the polypeptide complex described herein and a pharmaceutically acceptable carrier.

In yet another aspect, the present disclosure provides a conjugate comprises the polypeptide complex described herein, and a payload conjugated thereto, wherein the payload is selected from the group consisting of a radioactive label, a fluorescent label, an enzyme-substrate label, an affinity purification tag, a tracer molecule, an anticancer drug, an immune-related molecule, and a cytotoxic molecule.

In yet another aspect, the present disclosure provides a composition comprising the polypeptide complex or the conjugate described herein, and a pharmaceutically acceptable carrier.

In yet another aspect, the present disclosure provides a method of treating or preventing from a disease, condition, or symptom. In some embodiments, the method comprises administering to a subject in need thereof a therapeutically effective amount of the polypeptide complex described herein, the pharmaceutical composition described herein, the conjugate described herein, or the composition described herein.

In some embodiments, the disease is selected from the group consisting of a cancer, an inflammatory disease, an infectious or parasitic disease, a cardiovascular disease, eye disease, central nerves system (CNS) disease, an injury, metabolic disease, autoimmune disease, or a coagulation disorder. In some embodiments, the CNS disease is neuropathy, a neuropsychiatric condition, neuroblastoma, glioblastoma, or Alzheimer Disease.

In yet another aspect, the present disclosure provides a method of detecting presence or level of an antigen. In some embodiments, the method comprises contacting a sample suspected of containing the antigen with the polypeptide complex described herein and determining the formation of a complex between the antigen and the polypeptide complex.

### BRIEF DESCRIPTION OF THE DRAWINGS

FIG. 1 depicts each polypeptide fragment of an exemplary polypeptide complex which is constructed by two pairs of monoclonal antibodies and a pair of Fc regions.

FIG. 2 depicts diagrammatically the structure of an exemplary polypeptide complex constructed by the method of the present invention (Method B).

FIG. 3 further depicts the structure of an exemplary polypeptide complex constructed by the method of the present invention (Method B) with the Fab fragments from both monoclonal antibodies subdivided into VH/VL and CH1/CL pairs.

FIGS. 4A and 4B further depict the structure of an exemplary polypeptide complex constructed by the method of the present invention (Method B) with defined VH/VL and CH1/CL pairs of Fab fragments from both monoclonal antibodies. FIGS. 4C-4E depict the antibody structures with B1-A2 mispairing (FIG. 4C), B2-A1 mispairing (FIG. 4D), and B1-A2 and B2-A1 mispairing (FIG. 4E), respectively.

FIGS. 5A and 5B depict the diagram of the structure of an exemplary bispecific antibody constructed by the conventional approach (Method A). FIGS. 5C-5E depict the antibody structures with B1-A2 mispairing (FIG. 5C), B2-A1 mispairing (FIG. 5D), and B1-A2 and B2-A1 mispairing (FIG. 5E), respectively.

FIGS. 6A and 6B indicate the expression results of exemplary polypeptide complexes (FORMAT EX1-8 and FORMAT NEW1-8) analyzed by SDS-Page.

FIGS. 7A-7P indicate the expression results of exemplary polypeptide complexes (FORMAT EX1-8 and FORMAT NEW1-8) analyzed by SEC-HPLC.

FIG. 8 depicts the purity of the expressed polypeptide complex anti-CD20×CD3 (FORMAT NEW7) analyzed by SDS-Page.

FIG. 9 depicts the purity of the expressed polypeptide complex anti-CD20×CD3 (FORMAT NEW7) analyzed by CE-SDS.

FIG. 10 depicts the purity of the expressed polypeptide complex anti-CD20×CD3 (FORMAT NEW7) analyzed by SEC-HPLC.

FIG. 11 depicts the purification profile of the expressed polypeptide complex anti-CD20×CD3 (FORMAT NEW7) conducted by Affinity Chromatography.

FIG. 12 shows the SEC-HPLC purity analysis of the expressed polypeptide complex anti-CD20×CD3 (FORMAT NEW7) purified by Affinity Chromatography.

FIG. 13 shows the SDS-Page purity analysis of the expressed polypeptide complex anti-CD20×CD3 (FORMAT NEW7) purified by Affinity Chromatography.

FIG. 14 depicts the purification profile of the expressed polypeptide complex anti-CD20×CD3 (FORMAT NEW7) under linear gradient elution by CEX.

FIG. 15 shows the SDS-Page purity analysis of the expressed polypeptide complex anti-CD20×CD3 (fractions C01-C04) purified by the CEX.

FIG. 16 shows the SEC-HPLC purity analysis of the expressed polypeptide complex anti-CD20×CD3 (fractions C01-C04) purified by the CEX.

FIG. 17 shows the sequences of exemplary scaffold region pairs used in the bispecific antibodies.

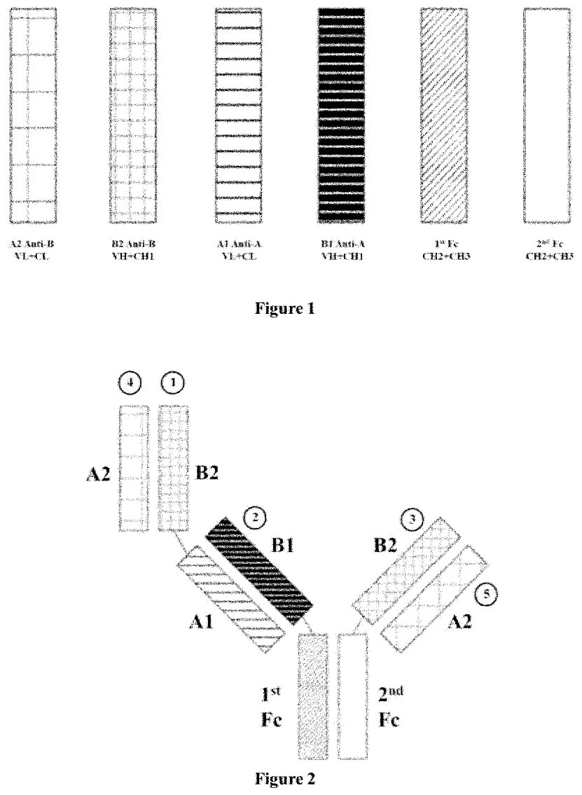

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events.

Furthermore, not all illustrated acts or events are required to implement a methodology in accordance with the present invention.

### I. Definitions and Abbreviations

Before the detailed description of the inventions is provided, the following are noted and defined.

All the description provided herein is merely intended to illustrate various embodiments of the inventions provided in the present disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein.

All references cited in the present disclosure, including patent applications, issued patents, published articles or other publications, are incorporated by reference in their entirety, which are for the purpose of providing methodologies that might be used in connection with the description provided herein. With respect to any term that is presented in one or more publications that is similar to, or identical with, a term that has been expressly defined in this disclosure, the definition of the term as expressly provided in this present disclosure will control in all respects.

All technical and scientific terms used, unless expressly defined otherwise, in this present disclosure, are generally deemed to have the same meaning as commonly understood to one of ordinary skill in the art to which this disclosure belongs.

As used herein, i.e., throughout the whole disclosure, the articles “a,” “an,” and “the” are to be construed to mean “one or more” or “at least one” unless specified otherwise. By way of example, “a polypeptide complex” means one polypeptide complex or more than one polypeptide complex.

As used herein, the terms “about,” “approximately,” “around” or alike, refer to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the terms “comprise,” “comprises,” “comprising,” “include,” “includes,” “including,” “contain,” “contains,” “containing”, “have,” “has,” “having” and the like, are synonymous and are used inclusively, in an open-ended fashion, and do not exclude additional elements, features, steps, acts, operations, and so forth.

As used herein, the term “or” is used in its inclusive sense (and not in its exclusive sense) so that when used, for example, to connect a list of elements, the term “or” means one, some, or all of the elements in the list.

As used herein, the phrase “at least one” means one or more, i.e. 1, 2, 3, 4, 5, 6, 7, 8, 9, 10, or more. A phrase referring to “at least one of” a list of items is construed to refer to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiment requires at least one of X, at least one of Y, and at least one of Z to each be present.

As used herein, the references “one embodiment,” “an embodiment,” “a particular embodiment,” “a related embodiment,” “a certain embodiment,” “an additional embodiment,” “some embodiments,” “certain embodiments,” or “a further embodiment” or combinations thereof, are to be understood to mean that a particular feature, structure or characteristic described in connection with this particular embodiment is included in at least one embodiment of the present disclosure. Thus, the presences or appearances of the foregoing phrases in various places throughout this disclosure are not necessarily all referring to the same embodiment. Furthermore, the particular features, structures, or characteristics may be combined in any suitable manner in one or more embodiments.

Conditional language used herein, such as, among others, “can,” “could,” “might,” “may,” “e.g.,” and the like, unless stated expressly otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

In this section, definitions for some general terms are provided. Definition for other terms may be found in other sections of the disclosure that follow.

The terms “polypeptide”, “peptide”, and “protein” are used interchangeably herein to designate a linear series of amino acid residues connected one to the other by peptide bonds, which includes proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

As used herein, the term “amino acid” refers to a building block of a protein, a peptide, a polypeptide or an amino acid polymer, and the term “amino acid” further refers to a naturally occurring or synthetic amino acid, as well as any amino acid analog and amino acid mimetics that functions in a manner similar to the naturally occurring amino acid. Naturally occurring amino acids are those encoded by the genetic codes, as well as those amino acids that are later modified, e.g., hydroxyproline, gamma-carboxyglutamate, and O-phosphoserine. As used within this application, naturally occurring amino acids include the group of naturally occurring carboxy alpha-amino acids comprising alanine (three letter code: Ala, one letter code: A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V).

As used herein, the term “domain” refers to a globular structure formed by one or more regions of one or more polypeptide chains comprising peptide loops (e.g., comprising 3 to 4 peptide loops) that are stabilized, for example, by β-pleated sheet and/or intrachain disulfide bond(s). Examples may include a Fab domain (see below for more details). It is noted that in the present disclosure, the two terms “domain” and “region” may be used interchangeably.

As used herein, the term “nucleic acid,” “nucleic acid molecule,” “nucleotide,” “polynucleotide” or alike, is construed to refer to a nucleotide polymer of any length, can include both DNA and RNA, can be single-stranded or double-stranded, and includes analogs of naturally occurring polynucleotides in which one or more nucleotides are modified over naturally occurring nucleotides.

The term “antibody” as used herein encompasses any immunoglobulin, monoclonal antibody, polyclonal antibody, chimeric antibody, humanized antibody, multispecific antibody, bispecific antibody, bivalent antibody or multivalent antibody that binds to a specific antigen. In the following, a description of an antibody, as well as terms relevant thereto, is provided in more detail.

In mammals such as human, depending on the different types of heavy chains present in the immunoglobulins, there are five different classes/isotypes (i.e. IgA, IgD, IgE, IgG, and IgM, corresponding to the five Ig heavy chain types α, δ, ε, γ, and μ, respectively) of antibodies, which typically have different molecular and biological properties, functional locations, physiological functionalities, and pathological implications in diseases. Certain antibody classes may further include subclasses. For example, in human, IgA may include IgA1 and IgA2 subclasses, and IgG may include four subclasses denoted as IgG1, IgG2, IgG3, and IgG4, respectively. With an immunoglobulin monomer as its basic functional unit, a mammalian antibody may exist as a monomer (e.g. IgD, IgE, and IgG), a dimer (IgA), a tetramer (IgM), or a pentamer (IgM). In mammals, two types of light chain exist, including kappa (κ) chain and lambda (λ) chain.

Within the basic immunoglobulin unit, a native or naturally occurring antibody such as IgG generally includes two identical heavy (H) chains and two identical light (L) chains. Each light chain is linked to a heavy chain by one covalent disulfide bond or linkage formed between a pair of cysteine residues present respectively in the each light chain and the heavy chain, and the two heavy chains are further linked to one another through several disulfide bonds formed between cysteine residues in each heavy chain. The tetramer thus formed substantially takes a Y-like shape for the antibody, with the end of each fork arm containing an identical antigen-binding site (i.e. paratope) that interacts specifically with a corresponding epitope of the antigen.

More specifically, in a native antibody, in a N-terminal-to-C-terminal direction, each heavy chain includes a variable region (VH, or HCVR), followed by three or four constant regions (“CHs”, with IgA, IgD, IgG containing three CH regions CH1, CH2 and CH3; and IgE and IgM containing four CH regions CH1, CH2, CH3 and CH4), and each light chain includes a variable region (VL, or LCVR) and a constant region (CL). In the Y-shaped antibody, the variable region of each light chain (i.e. the VL region) aligns or associates with the variable region of its pairing heavy chain (i.e. the VH region) to together form an antigen-binding site for the antibody.

The term “variable region” or “VR” as used herein means the region in heavy chain or light chain of an antibody that are responsible for antigen binding. In a native antibody, the heavy chain variable region (VH or HCVR) contains three highly variable loops called “complementarity determining regions” (CDRs), i.e., heavy (H) chain CDRs including HCDR1, HCDR2, HCDR3, and the light chain variable region (VL or LCVR) contains three light (L) chain CDRs including LCDR1, LCDR2, and LCDR3. CDR boundaries for antibodies may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol. Biol. Dec 5; 186(3):651-63 (1985); Chothia, C. and Lesk, A. M., J. Mol. Biol., 196,901 (1987); Chothia, C. et al., Nature. Dec. 21-28; 342(6252):877-83 (1989); Kabat E. A. et al., National Institutes of Health, Bethesda, Md. (1991)). The three CDRs are interposed between flanking stretches known as “framework regions” (FRs), which are more highly conserved than the CDRs and form a scaffold to support the hypervariable loops. In a native antibody, each VH and VL comprises four FRs, and the CDRs and FRs are arranged from amino terminus to carboxyl terminus in the order: FR1, CDR1, FR2, CDR2, FR3, CDR3, FR4. However, it should be understood that the term “variable region” as used herein does not necessarily need to include all of the three CDRs or all of the four FRs, and should be construed to encompass any variant or derivative of a native variable region from a native antibody, as long as such variant or derivative retains antigen-binding activity.

The term “constant region” or “constant moiety” as used herein means the region in heavy chain or light chain of an antibody that are not directly involved in antigen binding. It should be understood that the term “constant region” or “constant moiety” as used herein does not necessarily need to include the full length of a native constant region of a native antibody, and should be construed to encompass any variant or derivative of such a native constant region or constant moiety, as long as such variant or derivative retains the ability to, for example, support stability of the antigen-binding domain, or retains the intended biological function such as effector functions such as secretion, transplacental mobility, Fc receptor binding, complement binding, and the like.

The term “CL region” refers to the constant region of an immunoglobulin light chain that is adjacent to the VL region. CL region may span from about EU position 108 to about EU position 216 in an immunoglobulin light chain. In a native antibody, the constant region of each light chain (i.e. the CL region) associates with the first constant region of a pairing heavy chain (i.e. the CH1 region).

The term “CH1 region” as used herein encompasses the first (most amino terminal) constant region of an immunoglobulin heavy chain that extends from, about EU position 118 to at least about EU position 220 (e.g. can be extended to EU position 221, and so on). The CH1 region is adjacent to the VH region, and is amino terminal to the hinge region of an immunoglobulin heavy chain molecule.

The term “hinge region” in terms of an antibody includes the portion of a heavy chain molecule that joins the CH1 region to the CH2 region. The length of hinge region may vary depending on the defined boundaries of the CH1 region and of the CH2 region. The hinge region is normally flexible, thus allowing the two N-terminus antigen binding regions to move independently.

The term “CH2 region” as used herein refers to the portion of a heavy chain immunoglobulin molecule that extends, e.g., from about EU position 231 to EU position 340.

The term “CH3 region” as used herein refers to the portion of a heavy chain immunoglobulin molecule that extends approximately 110 residues from N-terminus of the CH2 domain, e.g., from about EU position 341 to EU position 445, 446 or 447. The CH3 domain typically forms the C-terminal portion of the antibody like IgG, IgA, and IgD. In some immunoglobulins like IgE and IgM, however, additional domains may extend from CH3 domain to form the C-terminal portion of the molecule (e.g. the CH4 domain in the chain of IgM and the $ chain of IgE).

“Fc” as used herein refers to a portion derived from an antibody, for example, IgG, mainly composed of the second (CH2) and third (CH3) constant regions of a first heavy chain bound to the CH2 and CH3 of a second heavy chain via one or more covalent bonds which are non-peptide bonds, for example, via disulfide bonding. The Fc portion of the antibody is responsible for various effector functions such as antibody-dependent cellular cytotoxicity (ADCC) and complement-dependent cytotoxicity (CDC), etc., but does not function in antigen binding.

An “antigen” or “Ag” as used herein refers to a compound, composition, peptide, polypeptide, protein or substance (e.g., polypeptide, carbohydrate, nucleic acid, lipid, or other naturally occurring or synthetic compound) that can be specifically recognized and bound by a component of the immune system, e.g., an antibody. As used herein, the term “antigen” encompasses antigenic epitopes, e.g., fragments of an antigen which are antigenic epitopes. The term “antigen” and “target” are used interchangeably in the present disclosure.

“Fv” with regard to an antibody refers to the smallest fragment of the antibody to bear the complete antigen-binding site. An Fv fragment consists of the variable domain of a single light chain bound to the variable domain of a single heavy chain. A number of Fv designs have been provided, including dsFvs, in which the association between the two domains is enhanced by an introduced disulfide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerized to form diabodies and triabodies (Maynard et al., Annu Rev Biomed Eng 2 339-376 (2000)).

“Fab” as used herein refers to a single antigen-binding domain derived from an antibody, where the domain has a single heavy chain fragment associated with a single light chain fragment via one or more covalent bonds which are non-peptide bonds. In some embodiments, the single heavy chain fragment in the Fab domain comprises an HCVR and a CH1 region. In some embodiments, the single light chain fragment in the Fab domain comprises an LCVR and a CL domain. In some embodiments, the CH1 region associates with the HCVR by a covalent bond such as a disulfide bond. In a native antibody, the Fab domain corresponds substantially to one arm of the antibody, typically retains the ability to recognize and bind to its corresponding antigen.

As used herein, the term “multispecific antibody” refers to an artificial or engineered antibody that can simultaneously bind to at least two different epitopes. A bispecific antibody is substantially a type of a multispecific antibody. In addition, other multispecific antibodies may include trispecific antibodies, which have three different antigen-binding specificities, tetraspecific antibodies, which have four different antigen-binding specificities, and so on.

As used herein, the term “bispecific antibody” refers to an antibody that comprises two physically separable antigen-binding domains which differ from one another in their antigen specificity. Usually, a bispecific antibody is an artificial antibody which has fragments derived from two different monoclonal antibodies and is capable of binding to two different epitopes. The two epitopes may present on the same antigen, or they may present on two different antigens. It is in contrast to a naturally occurring antibody which has two physically separable antigen-binding moieties that are structurally identical and thus have the same antigen specificity.

Throughout the disclosure, the numbers indicating the positions of amino acid residues in a constant region of an antibody, such as those in the heavy chain constant region 1 (CH1) and the light chain constant region (CL) in a constant moiety, are based on the EU numbering, as described in Edelman, G. M. et al., Proc. Natl. Acad. USA, 63, 78-85 (1969). As indicated, some positions use IMGT numbering or Kabat index, as described in Kabat et al., Sequences of Proteins of Immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015). These numberings are also available from the IMGT scientific chart, accessible from the website of international ImMunoGeneTics information system.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

Antibody and fragments thereof according to the present disclosure encompass bispecific and multi-specific antibodies and fragments thereof. Bispecific or multi-specific antibodies may resemble single antibodies (or antibody fragments) but have two or more different antigen binding sites. Bispecific antibodies may have binding specificities for at least two different epitopes. Bispecific antibodies and fragments can also be in form of heteroantibodies. Heteroantibodies are two or more antibodies, or antibody binding fragments (e.g., Fab) linked together, each antibody or fragment having a different specificity.

The term “specific binding of an antibody” or “antigen-specific antibody” in the context of a characteristic of an antibody refers to the ability of an antibody to preferentially bind to a particular antigen that is present in a mixture of different antigens. In certain embodiments, a specific binding interaction will discriminate between desirable and undesirable antigens (or “target” and “non-target” antigens) in a sample, in some embodiments more than about 10 to 100-fold or more (e.g., more than about 1000- or 10,000-fold). In certain embodiments, the affinity between an antibody and antigen when they are specifically bound in an antibody antigen complex is characterized by a K_{D} (dissociation constant) of less than 10⁻⁶M, less than 10⁻⁷ M, less than 10⁻⁸ M, less than 10⁻⁹ M, less than 10⁻⁹ M, less than 10⁻¹¹M, or less than about 10⁻¹² M or less.

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or an antigen-binding fragment thereof, at least one promoter (e.g., SV40, CMV, EF-1α) operably linked to the nucleic acid sequence, and at least one selection marker.

The phrase “host cell” as used herein refers to a cell into which an exogenous polynucleotide and/or a vector can be or has been introduced.

Antibody conjugates are also provided. The terms “conjugated” and “joining” generally refer to a chemical linkage, either covalent or non-covalent that proximally associates one molecule with second molecule. The conjugates include any antibody of the present disclosure and an agent. The agent may be selected from a therapeutic agent, an imaging agent, a labeling agent, or an agent useful for therapeutic and/or labeling purposes.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from heart, liver, spleen, lung, kidney, skin or blood vessels of the subject.

### II. Novel Multispecific (or Bispecific) Polypeptide Complexes

In one aspect, the present disclosure provides a novel multispecific (or bispecific) polypeptide complex that is convenient to manufacture and purify, therefore can be provided with high purity and high yields.

Multispecific (or bispecific) antibodies are of great interest in the field of antibody engineering and have prospects of broad applications such as in anti-tumor immunotherapy. Different formats for bispecific antibodies have been developed, including IgG-like bispecific antibodies. IgG-like bispecific antibodies are bivalent, comprising two pairs of heavy chain (HC) and light chain (LC) derived from two different antibodies.

An IgG-like bispecific antibody provides two monovalent Fabs, each binding to a different antigen. This, however, can lead to decrease in affinity to each antigen and even loss of certain functions, when compared with conventional IgG antibodies that provide bivalent binding to a single antigen.

Another challenge relates to differentiating the binding affinity of an IgG-like bispecific antibody to the two target antigens. Depending on the intended mechanism of action (i.e., MOA) for the two target antigens, there could be different requirements on binding affinity to the two target antigens. For example, for CD3/CD20 bispecific antibodies, it is desirable to achieve nano-molar level affinity for CD20, but sub-micromolar level (for example 30-100 nm) for CD3. To increase affinity to a target of interest, an additional Fab domain targeting such a target can be fused to one of the heavy chains of the IgG-like bispecific antibody (see FIG. 5A). The structure of such a bispecific but trivalent antibody is referred to herein as 2:1 structure, that is, the binding to one antigen of interest is bivalent, while the binding to the other antigen remains monovalent. Such bivalent binding restores binding affinity to that antigen target, and in the meantime can confer affinity difference between the two antigen targets.

Despite of the benefits provided by a 2:1 structure bispecific antibody, there are some insurmountable challenges in its expression and production. For example, LC-HC binding instability and LC mispairing may occur during the assembling of such 2:1 structure bispecific antibodies. There may be different LC mispairing products (i.e. impurities) that are similar or undistinguishable from the target product (i.e. correct-paired) in terms of molecular weight as well as physical and chemical characteristics, making it difficult to separate or purify the target product from these impurities. This could result in low purity of the expressed target product and reduced yield.

The present disclosure provides a novel 2:1 structure bispecific antibody platform that can not only meet the requirements for the affinity difference between two antigen targets but is also much easier to purify than conventional 2:1 structure bispecific antibodies. This novel 2:1 structure is advantageous because the potential mispaired products (if any) are sufficiently different from the correct-paired product, for example, in molecular weight as well as in physical and chemical characteristics, making it easier to remove the mispaired products. Therefore, the bispecific antibody constructed according to this novel 2:1 structure platform can achieve higher purities and yields with relatively simple purification process.

### III. The Novel 2:1 Structure for the Multi-Specific Antibodies

The novel 2:1 structure provided herein is based on an IgG-like bispecific antibody, fused with an additional Fab domain binding to one of the two targets. Specifically, unlike conventional 2:1 structure bispecific antibody in which the additional Fab domain is fused to a heavy chain of the Ig-like bispecific antibody (see, the second polypeptide chain in FIG. 5A), the novel 2:1 structure provided herein has the additional Fab domain fused to a light chain of the IgG like bispecific antibody (see the first polypeptide chain in FIG. 2).

Specifically, as illustrated in FIG. 2, the novel 2:1 structure bispecific antibodies comprise a polypeptide complex composed of five polypeptide chains, including: 1) a first polypeptide chain comprising a fusion polypeptide comprising a first targeting binding fragment A1 and a second target binding fragment B2, 2) a second polypeptide comprising the first pairing fragment B1; 3) a third polypeptide comprising the second target binding fragment B2; 4) a fourth polypeptide comprising the second pairing fragment A2, and 5) a fifth polypeptide identical to the fourth polypeptide, i.e. also comprising the second target binding fragment A2. The A1 in the fusion polypeptide pairs with the B1 in the second polypeptide to form a first target binding domain, the B2 in the fusion polypeptide pairs with the A2 in the fourth polypeptide to form a second target binding domain, and the A2 in the fifth polypeptide pairs with the B2 in the third polypeptide to form another second target binding domain.

The uniqueness of the polypeptide complex provided herein at least lies in part in the fusion polypeptide (i.e. the first polypeptide chain) comprised therein, which is novel and not comprised in any of the conventional 2:1 structure bispecific antibodies. This fusion polypeptide makes it possible that any mispaired products would have significantly different molecular weights as well as physical and chemical characteristics from the target bispecific product.

In some embodiments, the fusion polypeptide comprises from C terminus to N terminus: a first target-binding fragment A1; a polypeptide linker; a second target-binding fragment B2; wherein, the polypeptide linker has a length that is sufficiently short to minimize potential intramolecular interaction between A1 and B2, the A1 is capable of pairing with a first pairing fragment B1, to form a first target binding domain; the B2 is capable of pairing with a second pairing fragment A2, to form a second target binding domain; and the A1 is configured to exhibit less binding to the B2 relative to the B1, and the B2 is configured to exhibit less binding to the A1 relative to the A2.

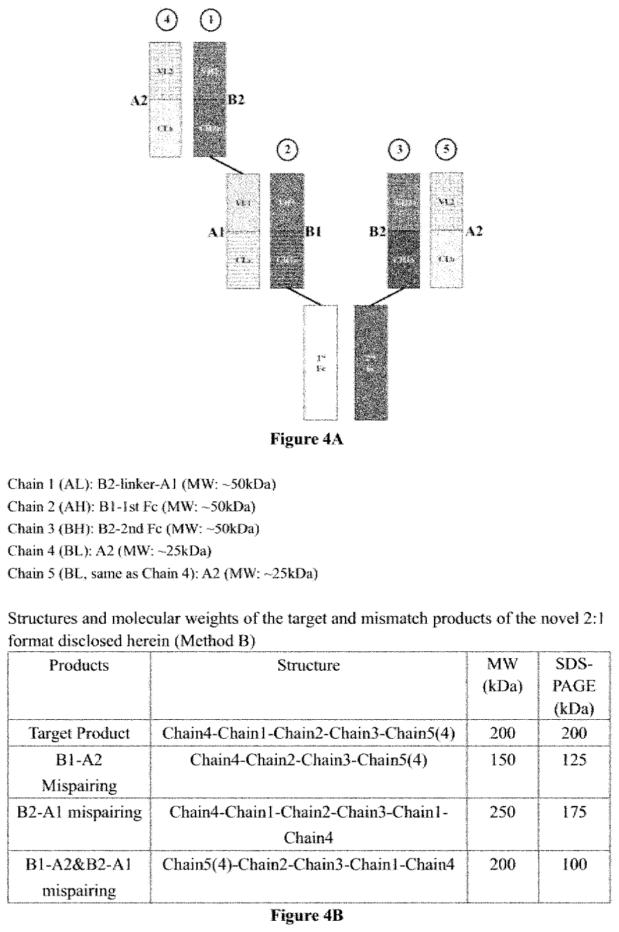

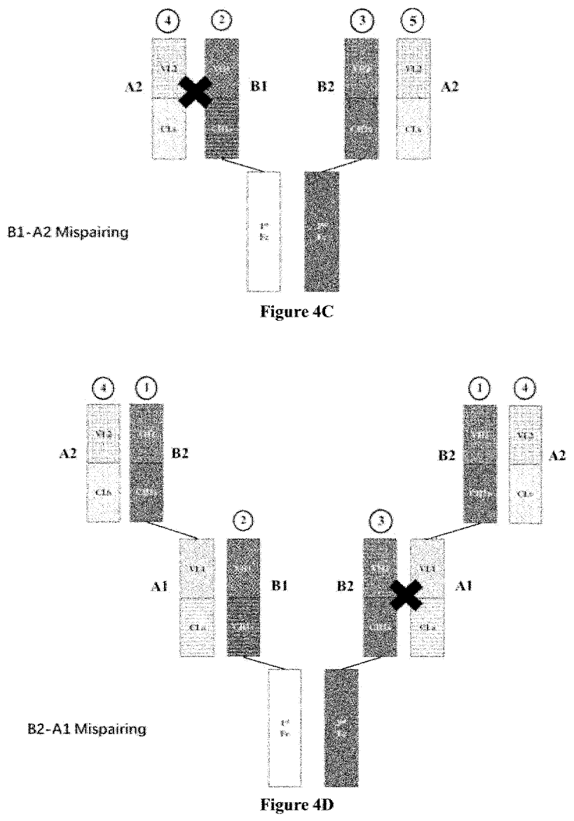

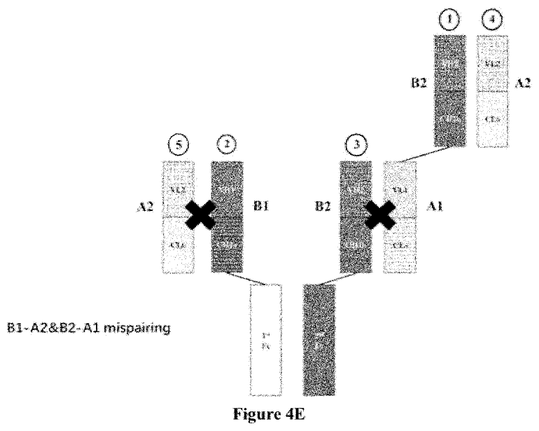

In the novel 2:1 structure as shown in FIG. 2, the two light chains are identical and therefore are useful to reduce unwanted mispairing. In an illustrative novel 2:1 structure provided herein (see FIG. 4A), a total of 3 different mispaired products (see FIGS. 4C-4E) are expected, which are estimated to have molecular weights of about 150 KDa, 250 KDa, and 200 KDa, respectively (see FIG. 4B), and therefore most of the potential mispaired products have different molecular weights from the correct paired product. Furthermore, these potentially mispaired products also are shown to have different physical and chemical characteristics from the correct paired bispecific product, and therefore can be readily removed by conventional purification methods.

In contrast, the conventional 2:1 structure bispecific antibody, as shown in FIG. 5A, has two different light chains that may potentially mispair with different heavy chains, rendering potentially a total of 5 different mispaired products (see FIGS. 5C-5E), which are estimated to all have the same molecular weights (i.e. 200 KDa) as the correct paired product (see FIG. 5B). These similar mispaired products are difficult to be separated from the target product, therefore compromising not only the purity but also the yield of the target product.

The novel 2:1 polypeptide complex provided herein binds to a first target and a second target. It has two antigen-binding domains that both bind to the second target, and a third antigen-binding domain that binds to the first target. In other words, it is bivalent for the second target and monovalent for the first target, and hence is referred to as “2:1”. The two antigen binding domains for the second target are located on different arms of the polypeptide complex provided herein.

In some embodiments, the target-binding domains in the polypeptide complex provided herein are formed by pairing of two polypeptide fragments. In certain embodiments, the first target binding domain is formed by the pairing of the first target-binding fragment A1 and the first pairing fragment B1 (i.e. A1/B1 pair). In certain embodiments, the second target binding domain is formed by the pairing of the second target-binding fragment A2 and the second pairing fragment B2 (i.e. A2/B2 pair).

In some embodiments, the first target-binding fragment A1 and the first pairing fragment B1 (i.e. A1/B1) can be paired by, for example, a disulfide bond, a hydrogen bond, electrostatic interaction, a salt bridge, or hydrophobic-hydrophilic interaction, a connecter, or the combination thereof, that is formed between A1 and B1, or more specifically, between at least two amino acid residues from A1 and B1.

Similarly, in some embodiments, the second target-binding fragment B2 and the second pairing fragment A2 (i.e. A2/B2) can be paired by, for example, a disulfide bond, a hydrogen bond, electrostatic interaction, a salt bridge, or hydrophobic-hydrophilic interaction, a connecter, or the combination thereof, that is formed between A2 and B2, or more specifically, between at least two amino acid residues from A2 and B2.

A “disulfide bond” refers to a covalent bond with the structure R—S—S—R′. The amino acid cysteine comprises a thiol group that can form a disulfide bond with a second thiol group, for example from another cysteine residue. The disulfide bond can be formed between the thiol groups of two cysteine residues residing respectively on the two polypeptide chains, thereby forming an interchain disulfide bond. Disulfide bond can be formed between known scaffold polypeptide fragments, such as antibody CH1 domain and CL domain, or TCR constant regions (such as TCR alpha/TCR beta, TCR gamma/TCR delta), to name just a few.

Electrostatic interaction is non-covalent interaction and is important in protein folding, stability, flexibility and function, including ionic interactions, hydrogen bonding and halogen bonding. Electrostatic interactions can be formed in a polypeptide, for example, between Lys and Asp, between Lys and Glu, between Glu and Arg, or between Glu, Trp on the first polypeptide chain and Arg, Val or Thr on the second polypeptide chain.

A salt bridge is close-range electrostatic interactions that mainly arises from the anionic carboxylate of either Asp or Glu and the cationic ammonium from Lys or the guanidinium of Arg, which are spatially proximal pairs of oppositely charged residues in native protein structures. Charged and polar residues in largely hydrophobic interfaces may act as hot spots for binding. Among others, residues with ionizable side chains such as His, Tyr, and Ser can also participate the formation of a salt bridge.

A hydrophobic interaction can be formed between one or more Val, Tyr and Ala on one polypeptide chain and one or more Val, Leu, and Trp on the second chain, or His and Ala on the first chain and Thr and Phe on another polypeptide chain.

A hydrogen bond is formed by electrostatic attraction between two polar groups when a hydrogen atom covalently bound to a highly electronegative atom such as nitrogen, oxygen, or fluorine. A hydrogen bond can be formed, for example, a nitrogen group in Asn and an oxygen group in His, or an oxygen group in Asn and a nitrogen group in Lys.

In some embodiments, the cognate pairing of A1 and B1 as shown in the novel 2:1 structure of FIG. 4A is facilitated by a native or an introduced non-native disulfide bond, whereas a mispaired product (such as mispaired A1 and B2, or mispaired A2 and B1) would lack such a disulfide bond. Without wishing to be bound by any theory, it is expected that such a disulfide bond would be particularly advantageous to permit easy identification and/or characterization of presence of mispaired products, for example, by using conventional methods such as sodium dodecyl sulfate-polyacrylamide gel electrophoresis (SDS-PAGE). This is at least partly because the disulfide bonded cognate pairing products can withstand the SDS-PAGE treatment, whereas the mispaired products lacking such disulfide bonding would suffer from dissociation of mispaired polypeptide chains, resulting in difference in molecular weight as observed with SDS-PAGE for the cognate pairing products and the mispaired products, as illustrated in FIGS. 4B-4E. Furthermore, the novel 2:1 structure of FIG. 4A permits easy separation of mispaired products from the cognate paired products using relatively conventional purification methods such as affinity purification and/or ion exchange purification, thereby achieving high purity of the cognate paired product that is otherwise not possible or requiring tedious purification process.

In certain embodiments, the A1/B1 pair and/or the A2/B2 pair comprise an antibody Fv domain.

In some embodiments, in the fusion polypeptide or the polypeptide complex provided herein, the A1 comprises a first antibody variable region VA1 selected from VH1 or VL1, the B1 comprises a first pairing antibody variable region VB1 capable of pairing with VA1 to form the first target-binding domain, wherein the VB1 is selected from VH1 or VL1. In other words, the first target-binding domain comprises a first Fv domain comprising VH1 and VL1 associated together that is capable of binding to the first target.

In some embodiments, in the fusion polypeptide or the polypeptide complex provided herein, the B2 comprises a second antibody variable region VB2 selected from VH2 or VL2, and the A2 comprises a second pairing antibody variable region VA2 capable of pairing with VB2 to form the second target-binding domain, wherein the VA2 is selected from VH2 or VL2. In other words, the second target-binding domain comprises a second Fv domain comprising VH2 and VL2 associated together that is capable of binding to the second target.

In some embodiments, the VB1 comprises VH1, the VA1 comprises VL1, the VB2 comprises VH2, and the VA2 comprises VL2. In some embodiments, the VB1 comprises VL1, the VA1 comprises VH1, the VB2 comprises VL2, and the VA2 comprises VH2.

In some embodiments, the first Fv domain or the second Fv domain is crossed. In some embodiments, the VB1 comprises VH1, the VA1 comprises VL1, the VB2 comprises VL2, and the VA2 comprises VH2. In some embodiments, the VB1 comprises VL1, the VA1 comprises VH1, the VB2 comprises VH2, and the VA2 comprises VL2.

The Fv domains can be associated by any suitable means known in the art, for example without limitation, by a non-native disulfide bond introduced in the Fv domain, or by a non-native electrostatic interaction introduced in the Fv domain. In some other embodiments, the Fv domains can be associated by scaffold domains fused to the Fv domains. For example a scaffold region can be fused to one chain of the Fv domain, and a pairing scaffold region can be fused to the other chain of the Fv domain. Binding of the scaffold region to the pairing scaffold region can allow association of the VH region and the VL region, thereby forming the Fv domain that is capable of binding to the target antigen.

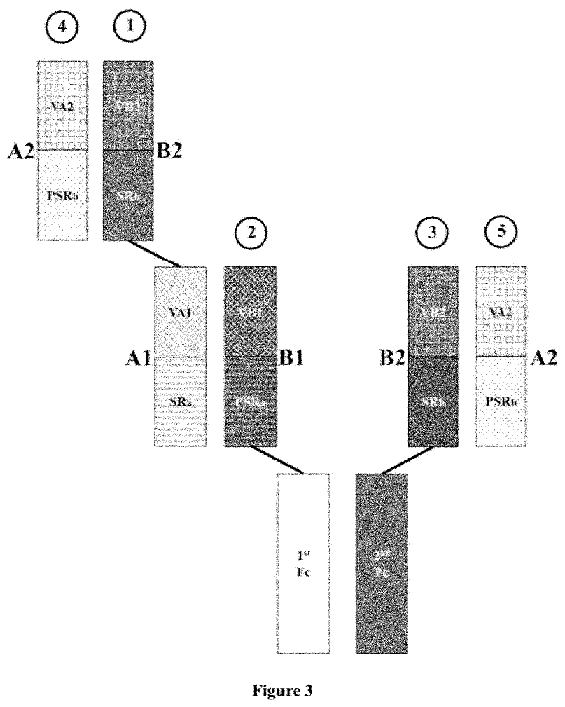

In some embodiments, in the polypeptide complex disclosed herein, the A1 further comprises a first scaffold region SRₐ operably linked to the VA1, and the B2 further comprises a second scaffold region SR_{b} operably linked to the VB2, wherein the SRₐ and the SR_{b} are configured such that pairing between the SRₐ and the SR_{b} is discouraged. In some embodiments, in the polypeptide complex disclosed herein, the B1 further comprises a first pairing scaffold region PSRₐ that is operably linked to the VB1 and is capable of binding to the SRₐ, and wherein the A2 further comprises a second pairing scaffold region PSR_{b} that is operably linked to the VA2 and is capable of binding to the SR_{b}. Binding of SRₐ and PSRₐ forms the first scaffold domain, and binding of SR_{b} and PSR_{b} forms the second scaffold domain. An illustrative drawing is shown in FIG. 3.

Any suitable binding partners can be used as the scaffold regions. In certain embodiments, the scaffold region can be selected from antibody CH1 domain and CL domain, pairing TCR constant regions (such as TCR alpha/TCR beta, TCR gamma/TCR delta), or PRD (proline rich domain) and SH3 domain, or obscurin and titin.

TCR belongs to the immunoglobulin superfamily, and is similar to a half antibody with a single heavy chain and a single light chain. Native TCR consists of two polypeptide chains, and has in general two types: one consists of alpha and beta chains (i.e. alpha/beta TCR), and the other consists of gamma and delta chains (i.e. gamma/delta TCR). The two TCR chains are linked by disulfide bonds formed between constant regions in the extracellular portion of the TCR chains. In other words, disulfide bonds are formed between TCR constant region alpha and TCR constant region beta, or between TCR constant region gamma and TCR constant region delta.

The SH3 domain, short for SRC Homology 3 Domain, is a small protein domain of about 60 amino acid residues, which was initially was described as a conserved sequence in the viral adaptor protein v-Crk. The classical SH3 domain is usually found in proteins that interact with other proteins and mediate assembly of specific protein complexes, typically via binding to proline-rich domains (i.e., PRD) in their respective binding partner (Liubov V Gushchina et al., J Biomol Struct Dyn. 2011 December; 29(3):485-95.).

Titin (also called connectin) is a protein that in humans is encoded by the TTN gene. Titin is a giant protein, greater than 1 μm in length, that functions as a molecular spring which is responsible for the passive elasticity of muscle. Obscurin (~800 kDa), the newest member of the titin family, was initially identified as a ligand of a Zdisk portion of titin, although it primarily localizes to the sarcomeric M-band in mature muscle. The giant muscular proteins titin and obscurin bind to each other at the Zdisk during muscle development. This interaction helps stabilize and organize the sarcomere; ablation of this binding leads to muscular dystrophy (Allyn G Letourneau et al., Protein Pept Lett. 2018; 25(11):973-979.). Specifically, the titin Ig-like 152 domain of titin interacts with the obscurin Ig-like 1 domain of obscurin, and they can form a complex. In some previously reported studies, a titin T-chain and an obscurin O-chain can be used to substitute the CH1 and CL regions of an antibody, wherein titin T-chain is a peptide containing titin Ig-like 152 domain with a length of 78-118 amino acids, or functional variants thereof, while obscurin O-chain is a peptide containing obscurin Ig-like 1 domain with a length of 87-117 amino acids, or functional variants thereof (see, e.g., WO2021/139757A, which is incorporated herein to its entirety).

Suitable pairs of scaffold regions useful in the present invention (i.e., the pair of SRₐ/PSRₐ or the pair of SR_{b}/PSR_{b}) include: a) a pair of heavy chain constant region 1 (CH1) and light chain constant region (CL); b) a pair of T cell receptor (TCR) constant region alpha (Calpha) and TCR constant region beta (Cbeta); c) a pair of TCR constant region gamma (Cgamma) and TCR constant region delta (Cdelta); d) a pair of ligand-binding domain of a receptor and the ligand; e) a pair of PRD (proline rich domain) and SH3 domain; f) a pair of obscurin and titin, or the like. It should be understood that, as used herein throughout the disclosure, the expression “a pair of X and Y” or “the X/Y pair” does not intend to limit the sequential order. For example, by stating that “the pair of SRₐ/PSRₐ can be a pair of CH1 and CL”, it is intended to mean that the SRₐ can be either CH1 or CL, and accordingly, when the SRₐ is CH1 then the PSRₐ can be CL, or when the SRₐ is CL then the PSRₐ can be CH1.

In some embodiments, the SRₐ and the PSRₐ are paired by a first disulfide bond. In such embodiments, mispairing of the SRₐ and the PSR_{b} or the SR_{b} and the PSRₐ would lack the first disulfide bond. In some embodiments, the SR_{b} and the PSR_{b} are paired by a second disulfide bond. In such embodiments, mispairing of the SRₐ and the PSR_{b} or the SR_{b} and the PSRₐ would lack the second disulfide bond. Any suitable pairs of scaffold regions capable of pairing by a disulfide bond can be used as the pair of SRₐ and the PSRₐ or the pair of SR_{b}/PSR_{b}. For example, antibody CH1 domain and CL domain can be paired by a disulfide bond; TCR constant regions (such as Calpha and Cbeta, or Cgamma and Cdelta) can be paired by a disulfide bond; and obscurin and titin can be paired by a disulfide bond.

In some embodiments, the pair of SRₐ/PSRₐ comprises a pair of a CH1 domain CH1a and a CL domain CLa, and the pair of SR_{b}/PSR_{b} comprises a pair of a CH1 domain CH1b and a CL domain CLb. An illustrative drawing is shown in FIG. 4A. In certain embodiments, the first target binding domain (formed by A1/B1 pair) and the second target binding domain (formed by A2/B2 pair) comprise antibody Fab domains.

In certain embodiments, the pairing between A1 and B1 can be formed between two amino acid residues in VA1 and VB1, in VH1 and VL1, in SRₐ and PSRₐ, or in CH1a and CLa. The pairing between A2 and B2 can be formed between two amino acid residues in VA2 and VB2, in VH2 and VL2, in SR_{b} and PSR_{b}, or in CH1b and CLb.

### IV. Configurations for Avoiding Mispairing

While it is intended that a target-binding polypeptide fragment binds to its pairing polypeptide fragment to form a target-binding domain (i.e. cognate pairing), mispairing nevertheless may happen between polypeptide fragments that are not intended to pair. For example, the first target-binding fragment A1 may mispair with the second target-binding fragment B2, or the first target-binding fragment A2 may mispair with the second target-binding fragment B1, thereby failing to form the intended target-binding domain.

For example, it is intended that VH1 pairs with VL1 to form the first target-binding domain, and VH2 pairs with VL2 to form the second target-binding domain, and such cognate pairing is intended to be facilitated. However, it is possible that VH1 may mispair with VL2, and VH2 may mispair with VL1, and such mispairing is preferably discouraged or reduced.

To facilitate the cognate pairing and hopefully reduce mispairing, the polypeptide fragments can be designed or engineered to contain certain configurations that helps achieve the purpose. In some embodiments, in the fusion polypeptide or the polypeptide complex disclosed herein, at least one of the pair of A1 and B1 (referred to as A1/B1) and the pair of A2 and B2 (referred to as A2/B2) contains at least one configuration capable of: a) facilitating cognate pairing between A1 and A2 and/or B1 and B2, and/or b) discouraging mispairing between B1 and A2 and/or between B2 and A1.

Any suitable methods known in the art can be used in the polypeptide complexes disclosed herein, to facilitate cognate pairing and/or reduce mispairing. Several strategies have been applied into designing orthogonal interfaces to facilitate cognate pairing. For example, Roche swapped the CH1 and CL domains and created the CrossMab platform (Schaefer et al. Proceedings of the National Academy of Sciences of the United States of America, 108(27), pp. 11187-11192 (2011)), MedImmune introduced non-native disulfide bond (Mazor et al. mAbs, 7(2), pp. 377-389 (2015)), Amgen further introduced electrostatic interactions in the CH1-CL region (Liu et al. Journal of Biological Chemistry, 290(12), pp. 7535-7562 (2015)), Eli Lilly (Lewis et al. Nature Biotechnology, 32(2), pp. 191-198 (2014)), Genentech (Dillon et al. mAbs, 9(2), pp. 213-230 (2017)) introduced mutations in both variable and constant domains, and Wuxi Biologics replaced one pair of CH1-CL region with T cell receptor (TCR) constant region (Guo et al., Protein Expr Purif, 173:105647 (2020)). These strategies have been shown to be useful to facilitate cognate pairing, and can be useful in the present disclosure.

In certain embodiments, in the fusion polypeptide or the polypeptide complex disclosed herein, both the pair of SRₐ/PSRₐ and the pair of SR_{b}/PSR_{b} are the same or of the same type, the pair of SRₐ/PSRₐ and/or the pair of SR_{b}/PSR_{b} are configured such that mispairing between SRₐ/PSR_{b} or between SR_{b}/PSRₐ is discouraged.

Any suitable means to discourage or reduce mispairing can be used. For example, the A1/B1 pair and the A2/B2 pair can be designed to incorporate crossed VA1/VB1 configuration (e.g. CrossMab), or to incorporate distinct pairing scaffold regions, or to incorporate crossed pairing scaffold regions, or to introduce at least one mutation in the otherwise identical pairing scaffold regions. More details are discussed below.

### A). Cross VA1/VB1 Configuration

In certain embodiments, the B1/A1 pair and the B2/A2 pair comprise antibody Fv domains and the Fv domains are crossed to discourage or reduce mispairing between B2 and A1, or between B1 and A2. Fv domains comprises a heavy chain variable domain VH and a light chain variable domain VL. The two Fv domains can be in crossed configuration such that when mispairing between B2 and A1 occurs and/or mispairing between B1 and A2 occurs, it would result in pairing of two VH domains or pairing of two VL domains, which are believed to be less stable than pairing between VH and VL, and hence less likely to form.

In some embodiments, the VB1 comprises VH1, the VA1 comprises VL1, the VB2 comprises VL2, and the VA2 comprises VH2. In some embodiments, the VB1 comprises VL1, the VA1 comprises VH1, the VB2 comprises VH2, and the VA2 comprises VL2. In these cross VA/VB1 configurations, when B2 mispairs with A1, or when B1 mispairs with A2, it would result in VH1 pairing with VH2, or VL1 pairing with VL2, which are believed to be less likely to form stable products.

In certain of these embodiments, the VB1 is operably linked to a first pairing scaffold region PSRₐ, and the VA1 is operably linked to the SRₐ, wherein the SRₐ binds to the PSRₐ to form the first pairing scaffold domain. In certain of these embodiments, the VB2 is operably linked to a second pairing scaffold region SR_{b}, and the VA2 is operably linked to the PSR_{b}, wherein the SR_{b} binds to the PSR_{b} to form the second pairing scaffold domain.

In some embodiments, the PSRₐ is CH1 domain CH1a, the SRₐ is CL domain CLa, the SR_{b} is CH1 domain CH1b, the PSR_{b} is CL domain CLb, and wherein: a) the VB1 comprises VH1, the VA1 comprises VL1, the VB2 comprises VL2, and the VA2 comprises VH2; orb) the VB1 comprises VL1, the VA1 comprises VH1, the VB2 comprises VH2, and the VA2 comprises VL2.

### B). Distinct Pairing Scaffold Regions

In certain embodiments, the B1/A1 pair comprises a first antibody Fv domain and a first pair of scaffold region (i.e. SRₐ/PSRₐ), and the B2/A2 pair comprises a second antibody Fv domain and a second pair of scaffold region (i.e. SR_{b}/PSR_{b}), wherein the first pair of scaffold region and the second scaffold regions are distinct, or are different types of scaffold regions. Accordingly, mispairing between A2 and B1, or mispairing between A1 and B2 would result in non-compatible scaffold regions that do not naturally bind to each other.

In some embodiments, the pair of the SRₐ and the PSRₐ is distinct from the pair of the SR_{b} and the PSR_{b}. In certain embodiments, the pair of SRₐ/PSRₐ or the pair of SR_{b}/PSR_{b} is selected from the group consisting of: a) a pair of heavy chain constant region 1 (CH1) and light chain constant region (CL); b) a pair of T cell receptor (TCR) constant region alpha (Calpha) and TCR constant region beta (Cbeta); c) a pair of TCR constant region gamma (Cgamma) and TCR constant region delta (Cdelta); d) a pair of ligand-binding domain of a receptor and the ligand; e) a pair of PRD (proline rich domain) and SH3 domain; and f) a pair of obscurin and titin. In some embodiments, the TCR constant region alpha (i.e., Calpha) comprises the amino acid sequence of SEQ ID NO: 97; and the TCR constant region beta (i.e., Cbeta) comprises the amino acid sequence of SEQ ID NO: 98.

In some embodiments, the pair of the SRₐ and the PSRₐ is a pair of CH1 and CL, and the pair of the SR_{b} and the PSR_{b} is a pair of: i) Calpha and Cbeta, ii) Cgamma and Cdelta, iii) ligand-binding domain of a receptor and the ligand; iv) PRD (proline rich domain) and SH3 domain, or v) obscurin and titin. In some embodiments, the pair of the SR_{b} and the PSR_{b} is a pair of CH1 and CL, and the pair of the SRₐ and the PSRₐ is a pair of: i) Calpha and Cbeta, ii) Cgamma and Cdelta, iii) ligand-binding domain of a receptor and the ligand, iv) PRD (proline rich domain) and SH3 domain, or v) obscurin and titin.

In some embodiments, the pair of the SRₐ and the PSRₐ is a pair of Calpha and Cbeta, and the pair of the SR_{b} and the PSR_{b} is a pair of: i) CH1 and CL, ii) Cgamma and Cdelta, iii) ligand-binding domain of a receptor and the ligand; iv) PRD (proline rich domain) and SH3 domain, or v) obscurin and titin. In some embodiments, the pair of the SR_{b} and the PSR_{b} is a pair of Calpha and Cbeta, and the pair of the SRₐ and the PSRₐ is a pair of: i) CH1 and CL, ii) Cgamma and Cdelta, iii) ligand-binding domain of a receptor and the ligand, iv) PRD (proline rich domain) and SH3 domain, or v) obscurin and titin.

In some embodiments, the pair of the SRₐ and the PSRₐ is a pair of Cgamma and Cdelta, and the pair of the SR_{b} and the PSR_{b} is a pair of: i) CH1 and CL, ii) Calpha and Cbeta, iii) ligand-binding domain of a receptor and the ligand; iv) PRD (proline rich domain) and SH3 domain, or v) obscurin and titin. In some embodiments, the pair of the SR_{b} and the PSR_{b} is a pair of Cgamma and Cdelta, and the pair of the SRₐ and the PSRₐ is a pair of: i) CH1 and CL, ii) Calpha and Cbeta, iii) ligand-binding domain of a receptor and the ligand, iv) PRD (proline rich domain) and SH3 domain, or v) obscurin and titin.

In some embodiments, the pair of the SRₐ and the PSRₐ is a pair of ligand-binding domain of a receptor and the ligand, and the pair of the SR_{b} and the PSR_{b} is a pair of: i) CH1 and CL, ii) Calpha and Cbeta, iii) Cgamma and Cdelta; iv) PRD (proline rich domain) and SH3 domain, or v) obscurin and titin. In some embodiments, the pair of the SR_{b} and the PSR_{b} is a pair of ligand-binding domain of a receptor and the ligand, and the pair of the SRₐ and the PSRₐ is a pair of: i) CH1 and CL, ii) Calpha and Cbeta, iii) Cgamma and Cdelta, iv) PRD (proline rich domain) and SH3 domain, or v) obscurin and titin.

In some embodiments, the pair of the SRₐ and the PSRₐ is a pair of PRD (proline rich domain) and SH3 domain, and the pair of the SR_{b} and the PSR_{b} is a pair of: i) CH1 and CL, ii) Calpha and Cbeta, iii) Cgamma and Cdelta; iv) ligand-binding domain of a receptor and the ligand, or v) obscurin and titin. In some embodiments, the pair of the SR_{b} and the PSR_{b} is a pair of PRD (proline rich domain) and SH3 domain, and the pair of the SRₐ and the PSRₐ is a pair of: i) CH1 and CL, ii) Calpha and Cbeta, iii) Cgamma and Cdelta, iv) ligand-binding domain of a receptor and the ligand, or v) obscurin and titin.

In some embodiments, the pair of the SRₐ and the PSRₐ is a pair of obscurin and titin, and the pair of the SR_{b} and the PSR_{b} is a pair of: i) CH1 and CL, ii) Calpha and Cbeta, iii) Cgamma and Cdelta; iv) ligand-binding domain of a receptor and the ligand, or v) PRD (proline rich domain) and SH3 domain. In some embodiments, the pair of the SR_{b} and the PSR_{b} is a pair of obscurin and titin, and the pair of the SRₐ and the PSRₐ is a pair of: i) CH1 and CL, ii) Calpha and Cbeta, iii) Cgamma and Cdelta, iv) ligand-binding domain of a receptor and the ligand, or v) PRD (proline rich domain) and SH3 domain.

In some embodiments, the cognate pairing of the scaffold regions are facilitated by introduction of non-native disulfide bonds. In such embodiments, the scaffold regions are CH1 and CL, Calpha and Cbeta or Cgamma and Cdelta.

### C). Cross Scaffold Regions

In certain embodiments, the first pair of scaffold regions and the second pair of the scaffold regions are of the same type of scaffold domain, but are configured such that mispairings between the first pair of scaffold regions and the second pair of the scaffold regions are discouraged.

In some embodiments, in the fusion polypeptide or the polypeptide complex provided herein, both the pair of SRₐ/PSRₐ and the pair of SR_{b}/PSR_{b} are of the same type, for example, both are CH1/CL domain, or Calpha/Cbeta domain, or Cgamma and Cdelta domain, and the like, but the pair of SRₐ/PSRₐ and/or the pair of SR_{b}/PSR_{b} are configured such that mispairing between SRₐ/PSR_{b} or between SR_{b}/PSRₐ is discouraged. Any suitable means can be used to discourage mispairing between two pairs of scaffold regions, for example, the pair of SRₐ/PSRₐ and the pair of SR_{b}/PSR_{b} can be configured to be crossed.

In certain embodiments, for example, both the pair of SRₐ/PSRₐ and the pair of SR_{b}/PSR_{b} are antibody constant domain formed by CH1/CL pairing. In certain embodiments, the A1/B1 pair and the A2/B2 pair comprise antibody Fab domains, and one of the VH/VL pairs in the Fab domains is crossed to discourage or reduce mispairing between B2 and A1, or between B1 and A2.

In some embodiments, the pair of SRₐ/PSRₐ comprises a CH1 domain CH1a and a CL domain CLa, and the pair of SR_{b}/PSR_{b} comprises a CH1 domain CH1b and a CL domain CLb. In some embodiments, the pair of CH1a/CLa and the pair of CH1b/CLb are configured to be crossed, such that when mispairing between B2 and A1 occurs and/or mispairing between B1 and A2 occurs, it would result in pairing of two non-pairing scaffold regions, such as CH1a and CH1b, or CLa and CLb, which are less stable than mispairing between CH1 and CL, and hence less likely to form.

In some embodiments, the VB1 comprises VH1, the VA1 comprises VL1, the VB2 comprises VH2, and the VA2 comprises VL2, and wherein: a) the PSRₐ is CL domain CLa, the SRₐ is CH1 domain CH1a, the SR_{b} is CH1 domain CH1b, the PSR_{b} is CL domain CLb; or b) the PSRₐ is CH1 domain CH1a, the SRₐ is CL domain CLa, the SR_{b} is CL domain CLb, the PSR_{b} is CH1 domain CH1b.

### D). Mutations Introduced in Otherwise Identical Pairing Scaffold Regions

In some embodiments, both the first pair of scaffold region and the second pair of the scaffold regions are the same type of scaffold domain, with one or more introduced mutations to reduce or discourage mispairing.

In some embodiments, the VB1 comprises VH1, the VA1 comprises VL1, the VB2 comprises VH2, and the VA2 comprises VL2, the PSRₐ is CH1 domain CH1a, the SRₐ is CL domain CLa, the SR_{b} is CH1 domain CH1b, the PSR_{b} is CL domain CLb, and wherein: a) the fusion polypeptide comprises an amino acid sequence of Formula (I): VH2-CH1b-Linker-VL1-CLa, b) the second polypeptide comprises an amino acid sequence of Formula (II): VH1-CH1a, c) the third polypeptide comprises an amino acid sequence of Formula (III): VH2-CH1b, d) the fourth and fifth polypeptides each comprising an amino acid sequence of Formula (IV): VL2-CLb; wherein the pair of CH1b/CLb and/or the pair of CH1a/CLa are configured such that mispairing between CH1b and CLa and/or between CH1a and CLb is discouraged.

In some embodiments, one or more mutations are introduced to the pair of CH1b/CLb and/or the pair of CH1a/CLa, discouraging the mispairing between CH1b and CLa and/or between CH1a and CLb. In certain embodiments the one or more mutations introduces a non-native binding interaction, such as for example, a non-native covalent bond, a non-native electrostatic interaction, a non-native salt bridge, a non-native hydrophobic-hydrophilic interaction, a non-native connecter, or any combination thereof. Preferably, the non-native binding interaction is introduced to either the pair of CH1b/CLb or the pair of CH1a/CLa, such that mispairing between the pair of CH1b/CLb and the pair of CH1a/CLa is discouraged.

In certain embodiments, at least one of the pair of CH1b/CLb and the pair of CH1a/CLa have one or more characteristics shown below: 1) having at least one non-natural disulfide bond that discourages mispairing between CH1b and CLa and/or between CH1a and CLb; 2) having one or more introduced amino acid mutations that form at least one or more introduced charged amino acid residues that discourages mispairing between CH1b and CLa and/or between CH1a and CLb; or 3) having one or more introduced amino acid mutations that form an orthogonal CH1-CL interface that discourages mispairing between CH1b and CLa or between CH1a and CLb.

### i) Disulfide Bonds Introduced in CH1/CL Regions

In certain embodiments, at least one of the pair of CH1b/CLb and the pair of CH1a/CLa have at least one non-natural disulfide bond that discourages mispairing between CH1b and CLa and/or between CH1a and CLb.

In some embodiments, a first CH1/CL pair and a second CH1/CL pair are selected from CH1b/CLb and CH1a/CLa. For example, the first CH1/CL pair can be CH1b/CLb and the second CH1/CL pair can be CH1ₐ/CLₐ; or alternatively, the first CH1/CL pair can be CH1ₐ/CLₐ and the second CH1/CL pair can be CH1b/CLb. In some embodiments, the first CH1/CL pair is associated by a first disulfide bond that is non-natural. In some embodiments, and the first CH1/CL pair lacks a natural disulfide bond or has a disrupted natural disulfide bond, for example, by mutation of the cysteine residues that form the natural disulfide bond.

In some embodiments, the second CH1/CL pair is associated by a second disulfide bond which is formed at a position different from the first disulfide bond. In some embodiments, the second disulfide bond formed in the second CH1/CL pair is a natural disulfide bond, or a non-natural disulfide bond.

In some embodiments, the first disulfide bond is formed by two cysteine residues introduced at a set of heavy chain-light chain EU positions selected from the group consisting of: a) heavy chain position 134 and light chain position 116, b) heavy chain position 141 and light chain position 116, c) heavy chain position 128 and light chain position 118, d) heavy chain position 126 and light chain position 121, e) heavy chain position 127 and light chain position 121, f) heavy chain position 126 and light chain position 124, g) heavy chain position 170 and light chain position 162, h) heavy chain position 171 and light chain position 162, and i) heavy chain position 173 and light chain position 162.

In some embodiments, the first disulfide bond is formed by two cysteine residues introduced at a set of heavy chain-light chain EU positions selected from the group consisting of: j) heavy chain position 133 and light chain position 209, k) heavy chain position 131 and light chain position 119, 1) heavy chain position 133 and light chain position 207, m) heavy chain position 170 and light chain position 176, n) heavy chain position 173 and light chain position 160, o) heavy chain position 133 and light chain position 117, and p) heavy chain position 129 and light chain position 121.

In some embodiments, the first disulfide bond is formed by two cysteine residues introduced at a set of heavy chain-light chain EU positions selected from the group consisting of: a) heavy chain EU position 126 and light chain EU position 121, b) heavy chain EU position 173 and light chain EU position 160, and c) heavy chain EU position 128—light chain EU position 118.

In some embodiments, the natural disulfide bond is between heavy chain EU position selected from 131, 219 and 220, and light chain EU position 214. In some embodiments, the natural disulfide bond is between heavy chain EU position 220 and light chain EU position 214.

In some embodiments, the first CH1/CL pair comprises a CH1 comprising substitution at EU position 126 for a cysteine residue, and substitution at EU position 220 for a non-cysteine residue; and a CL comprising substitution at EU position 121 for a cysteine residue, and substitution at EU position 214 for a non-cysteine residue.

In some embodiments, the CH1b comprises substitution at EU position 126 for a cysteine residue, and substitution at EU position 220 for a non-cysteine residue; and the CLb comprises substitution at EU position 121 for a cysteine residue, and substitution at EU position 214 for a non-cysteine residue. In some embodiments, the CH1b comprises the amino acid sequence of SEQ ID NO: 77, and/or the CLb comprises the amino acid sequence of SEQ ID NO: 79. In some embodiments, the CH1a comprises substitution at EU position 126 for a cysteine residue, and substitution at EU position 220 for a non-cysteine residue; and the CLa comprises substitution at EU position 121 for a cysteine residue, and substitution at EU position 214 for a non-cysteine residue. In some embodiments, the CH1a comprises the amino acid sequence of SEQ ID NO: 77, and/or the CLa comprises the amino acid sequence of SEQ ID NO: 79.

ii) Substitution with Charged Amino Acids

In certain embodiments, at least one of the pair of CH1b/CLb and the pair of CH1a/CLa have at least one or more introduced charged amino acid residues that discourages mispairing between CH1b and CLa and/or between CH1a and CLb.

In some embodiments, a first CH1/CL pair and a second CH1/CL pair are selected from CH1b/CLb and CH1a/CLa. For example, the first CH1/CL pair can be CH1b/CLb and the second CH1/CL pair can be CH1ₐ/CLₐ; or alternatively, the first CH1/CL pair can be CH1a/CLa and the second CH1/CL pair can be CH1b/CLb. In some embodiments, the first CH1/CL pair contains at least one substitution of a non-charged residue to a charged residue, and/or at least one substitution of a charged residue to an oppositely charged residue, such that the first CH1/CL pair contains a first pair of oppositely charged residues that favors pairing of first CH1/CL pair. In some embodiments, the first CH1/CL pair can contain a combination of substitutions, which together provide a first a first pair of oppositely charged residues that favors pairing of first CH1/CL pair.

In other words, a pair of oppositely charged residues can be introduced to the first CH1/CL pair, to facilitate cognate pairing between the CH1 domain and the CL domain in the first CH1/CL pair. For example, a charged residue can be introduced to replace a non-charged residue at a position in the CH1 (or CL), such that the introduced charged residue would form electrostatic interaction with another oppositely charged residue, which is already present or is to be introduced, in the CL (or CH1), so as to favor pairing of the first CH1/CL pair. For another example, an existing charged residue at a position in the CH1 (or CL) can be replaced with an oppositely charged residue, such that the charged residue after substitution would form electrostatic interaction with another oppositely charged residue, which is already present or is to be introduced, in the CL (or CH1), so as to favor pairing of the first CH1/CL pair. In certain embodiments, an existing charged residue in the CH1 or the CL may be replaced with a non-charged residue, to reduce potential interference with the electrostatic interaction between the first CH1/CL pair.

In some embodiments, the second CH1/CL pair contains at least one substitution of a non-charged residue to a charged residue, and/or at least one substitution of a charged residue to an oppositely charged residue, such that the second CH1/CL pair contains a second pair of oppositely charged residues that favors pairing of second CH1/CL pair, and optionally the first pair of oppositely charged residues and the second pair of oppositely charged residues discourages pairing of CH1a with CLb or CH1b and CLa.

In some embodiments, the first pair of oppositely charged residues and the second pair of oppositely charged residues are configured such that CH1a and CLb have both positive or both negative charged residues at the corresponding positions of the first and the second pair of oppositely charged residues, and/or CH1b and CLa have both positive or both negative charged residues at the corresponding positions of the first and the second pair of oppositely charged residues.

In some embodiments, the first pair of oppositely charged residues and/or the second pair of oppositely charged residues is introduced at a set of heavy chain-light chain EU positions selected from the group consisting of: a) heavy chain EU position 183 and light chain EU position 176, b) heavy chain EU position 183 and light chain EU position 133, c) heavy chain EU position 147 and light chain EU position 176, d) heavy chain EU position 141 and light chain EU position 116, e) heavy chain EU position 126 and light chain EU position 121, and f) heavy chain EU position 218 and light chain EU position 122.

In some embodiments, the first pair of oppositely charged residues and/or the second pair of oppositely charged residues is introduced at a set of heavy chain-light chain EU positions selected from the group consisting of: g) heavy chain EU position 147 and light chain EU position 131, h) heavy chain EU position 168 and light chain EU position 174, i) heavy chain EU positions 147 and 168, and light chain EU positions 131 and 174.

In some embodiments, the pair of oppositely charged residues comprises a positive-charged amino acid residue and a negative-charged amino acid residue, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

In some embodiments, the CH1b comprises substitution of lysine at EU position 147 for a negatively charged residue; the CLb comprises substitution of serine at EU position 176 for a positively charged residue; the CH1a comprises substitution of serine at EU position 183 for a positively charged residue; and the CLa comprises substitution of serine at EU position 176 for a negatively charged residue. In some embodiments, the CH1b comprises substitution K147D; the CLb comprises substitution S176K; the CH1a comprises substitution S183K; and the CLa comprises substitution S176D. In some embodiments, the CH1b comprises the amino acid sequence of SEQ ID NO: 88; the CLb comprises the amino acid sequence of SEQ ID NO: 90; the CH1a comprises the amino acid sequence of SEQ ID NO: 87; and the CLa comprises the amino acid sequence of SEQ ID NO: 92.

In some embodiments, the CH1a comprises substitution of lysine at EU position 147 for a negatively charged residue; the CLa comprises substitution of serine at EU position 176 for a positively charged residue; the CH1b comprises substitution of serine at EU position 183 for a positively charged residue; and the CLb comprises substitution of serine at EU position 176 for a negatively charged residue. In some embodiments, the CH1a comprises substitution K147D; the CLa comprises substitution S176K; the CH1b comprises substitution S183K; and the CLb comprises substitution S176D.

In some embodiments, the CH1a comprises the amino acid sequence of SEQ ID NO: 88; the CLa comprises the amino acid sequence of SEQ ID NO: 90; the CH1b comprises the amino acid sequence of SEQ ID NO: 87; and the CLb comprises the amino acid sequence of SEQ ID NO: 92.

In some embodiments, the first CH1/CL pair comprises CH1b and CLb.

In some embodiments, with respect to the CH1b, the amino acid residue at EU position 173 is replaced with a cysteine residue, the amino acid residue at EU position 183 is replaced with a positively charged residue, and the amino acid residue at EU position 220 is replaced with a non-cysteine residue, and with respect to the CLb, the amino acid residue at EU position 160 is replaced with a cysteine residue, the amino acid residue at EU position 176 is replaced with a negatively charged residue, and the amino acid residue at EU position 214 is replaced with a non-cysteine residue.

In some embodiments, with respect to the CH1b, the amino acid residue at EU position 173 is replaced with a cysteine residue, the amino acid residue at EU position 183 is replaced with a negatively charged residue, and the amino acid residue at EU position 220 is replaced with a non-cysteine residue, and with respect to the CLb, the amino acid residue at EU position 160 is replaced with a cysteine residue, the amino acid residue at EU position 176 is replaced with a positively charged residue, and the amino acid residue at EU position 214 is replaced with a non-cysteine residue.

In some embodiments, the CH1b comprises substitutions of V173C, S183K, and C220S, and the CLb comprises substitutions of Q160C, S176D, and C214S. In some embodiments, the CH1b comprises the amino acid sequence of SEQ ID NO: 103; and the CLb comprises the amino acid sequence of SEQ ID NO: 104.

In some embodiments, first CH1/CL pair comprises a combination of at least one non-natural disulfide bond and non-natural electrostatic interaction.

In some embodiments, the first CH1/CL pair comprises CH1a and CLa.

In some embodiments, with respect to the CH1a, the amino acid residue at EU position 173 is replaced with a cysteine residue, the amino acid residue at EU position 183 is replaced with a positively charged residue, and the amino acid residue at EU position 220 is replaced with a non-cysteine residue, and with respect to the CLa, the amino acid residue at EU position 160 is replaced with a cysteine residue, the amino acid residue at EU position 176 is replaced with a negatively charged residue, and the amino acid residue at EU position 214 is replaced with a non-cysteine residue. In some embodiments, the CH1a comprises substitutions of V173C, S183K, and C220S, and the CLa comprises substitutions of Q160C, S176D, and C214S. In some embodiments, the CH1a comprises the amino acid sequence of SEQ ID NO: 103; and the CLa comprises the amino acid sequence of SEQ ID NO: 104.

In some embodiments, with respect to the CH1a, the amino acid residue at EU position 173 is replaced with a cysteine residue, the amino acid residue at EU position 183 is replaced with a negatively charged residue, and the amino acid residue at EU position 220 is replaced with a non-cysteine residue, and with respect to the CLa, the amino acid residue at EU position 160 is replaced with a cysteine residue, the amino acid residue at EU position 176 is replaced with a positively charged residue, and the amino acid residue at EU position 214 is replaced with a non-cysteine residue.

iii) Formation of Orthogonal CH1/CL Interface

In certain embodiments, at least one of the pair of CH1b/CLb and the pair of CH1a/CLa have one or more introduced amino acid mutations that form an orthogonal CH1-CL interface that discourages mispairing between CH1b and CLa or between CH1a and CLb.

In certain embodiments, a first CH1/CL pair and a second CH1/CL pair are selected from CH1b/CLb and CH1a/CLa, and wherein the first CH1/CL pair comprises one or more introduced amino acid mutations that form an orthogonal CH1-CL interface.

In some embodiments, the at least one modification comprises one or more introduced amino acid mutations that form an orthogonal CH1-CL interface that favors pairing of CH1b and CLb, and optionally discourages pairing of CH1b with CLa or CH1a and CLb. In some embodiments, the at least one modification comprises one or more introduced amino acid mutations that form an orthogonal CH1-CL interface that favors pairing of CH1a and CLa, and optionally discourages pairing of CH1a with CLb or CH1b and CLa.

In some embodiments, the orthogonal CH1-CL interface comprises mutations at heavy chain EU positions H168A, F170G, and light chain EU positions L135Y, S176W. In some embodiments, the orthogonal CH1-CL interface comprises mutations at heavy chain EU positions H168A and F170G and light chain EU positions L135Y and S176W, wherein the heavy chain CH1 domain comprises the amino acid sequence of SEQ ID NO: 83, and the light chain CL domain comprises the amino acid sequence of SEQ ID NO: 95.

In some embodiments, the CH1a comprises substitutions of S183E, and the CLa comprises substitutions of V133K, the CH1b comprises substitutions of A141I, F170S, S181M, S183A, and V185A, and the CLb comprises substitutions of F116A, L235V, S174A, S176F, and T178V. In some embodiments, the CH1b comprises the amino acid sequence of SEQ ID NO: 68, and/or the CLb comprises the amino acid sequence of SEQ ID NO: 71.

In some embodiments, the CH1b comprises substitutions of S183E, and the CLb comprises substitutions of V133K, the CH1a comprises substitutions of A141I, F170S, S181M, S183A, and V185A, and the CLa comprises substitutions of F116A, L235V, S174A, S176F, and T178V. In some embodiments, the CH1a comprises the amino acid sequence of SEQ ID NO: 68, and/or the CLa comprises the amino acid sequence of SEQ ID NO: 71.

In some embodiments, the first CH1/CL pair comprises one or more introduced amino acid mutations that form orthogonal Fab designs at a set of heavy chain-light chain EU positions selected from the group consisting of: substitutions at heavy chain EU positions A141I, F170S, S181M, S183A, and V185A, and substitutions at light chain EU positions F116A, A235V, S174A, S176F, and T178V.

In some embodiments, the first CH1/CL pair comprises introduced amino acid mutations that form orthogonal Fab designs at a set of heavy chain-light chain EU positions selected from the group consisting of: substitutions at heavy chain EU positions A141I, F170S, S181M, S183A, and V185A, and substitutions at light chain EU positions F116A, A235V, S174A, S176F, and T178V; and the second CH1/CL pair comprises introduced amino acid mutations that form a pair of oppositely charged residues including S183E in CH1 and V133K in CL. In some embodiments, the CH1a comprises the amino acid sequence of SEQ ID NO: 68, the CLa comprises the amino acid sequence of SEQ ID NO: 71, the CH1b comprises the amino acid sequence of SEQ ID NO: 66, and/or the CLb comprises the amino acid sequence of SEQ ID NO: 73.

### E). Mutations Introduced in Fab Variable Regions Facilitating Specific Pairings

In addition to the scaffold regions, mutations can also be introduced in variable regions in any one of the VA1/VB1 and VA2/VB2 to discourage the mispairing of B1/A2 or B2/A1.

In some embodiments, in the fusion polypeptide or the polypeptide complex disclosed herein, a first VH/VL pair and a second VH/VL pair are selected from VH1/VL1 and VH2/VL2, and wherein the first VH/VL pair has at least one substitution of a non-charged residue to a charged residue, and/or at least one substitution of a charged residue to an oppositely charged residue, such that the first VH/VL contains a third pair of oppositely charged residues that favors pairing of first VH/VL pair.

In some embodiments, the second VH/VL pair has at least one substitution of a non-charged residue to a charged residue, and/or at least one substitution of a charged residue to an oppositely charged residue, such that the second VH/VL contains a fourth pair of oppositely charged residues that favors pairing of second VH/VL pair, and optionally the third pair of oppositely charged residues and the fourth pair of oppositely charged residues discourages pairing of VH1 with VL2 or VH2 and VL1.

In some embodiments, the third pair of oppositely charged residues and the fourth pair of oppositely charged residues are configured such that VH1 and VL2 have both positive or both negative charged residues, and/or VH2 and VL1 have both positive or both negative charged residues.

In some embodiments, the third pair of oppositely charged residues and/or the fourth pair of oppositely charged residues is introduced at a set of heavy chain-light chain EU positions selected from the group consisting of: a) heavy chain EU position 39: light chain EU position 38; b) heavy chain EU position 105: light chain EU position 43, c) heavy chain EU position 62: light chain EU position 1, or d) any combination thereof.

In some embodiments, the pair of oppositely charged residues comprises a positive-charged amino acid residue and a negative-charged amino acid residue, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

### F). Modification in Fc Regions

In some embodiments, in the polypeptide complex disclosed herein, the second polypeptide and the third polypeptide further comprise an operably linked first dimerization domain and an operably linked second dimerization domain, respectively, that are associated to form a dimer.

In some embodiments, the first dimerization domain and the second dimerization domain comprise CH3 domain of IgG. In some embodiments, the first dimerization domain and the second dimerization domain further comprise a hinge region.

In some embodiments, in the polypeptide complex disclosed herein, the second polypeptide comprises an operably linked first dimerization domain, and/or the third polypeptide further comprises an operably linked second dimerization domain. In some embodiments, in the polypeptide complex disclosed herein, the second polypeptide comprises an operably linked second dimerization domain, and/or the third polypeptide further comprises an operably linked first dimerization domain.

In some embodiments, in the polypeptide complex disclosed herein, the first dimerization domain comprises an operably linked first Fc region, and/or the second dimerization domain comprises an operably linked second Fc region. In some embodiments, the first Fc region and/or the second Fc region is derived from IgG1, IgG2, IgG3 or IgG4.

In some embodiments, the first Fc region and the second Fc region have different amino acid sequences and have at least one configuration that facilitates heterodimerization of the first Fc region and the second Fc region.

In certain embodiments, the polypeptide complex disclosed herein comprise one or more amino acid substitution(s) in the interface of the Fc region to facilitate and/or promote heterodimerization. These modifications comprise introduction of a protuberance into a first Fc region and a cavity into a second Fc region, wherein the protuberance can be positioned in the cavity so as to promote interaction of the first and second Fc polypeptides to form a heterodimer or a complex (also referred to as knob-into-hole structure). Methods of generating antibodies with these modifications are known in the art, e.g. as described in U.S. Pat. No. 5,731,168.

In some embodiments, a “knob” is generated by replacing one or more small amino acid side chains from the interface of the first antibody molecule with larger side chains (e.g., tyrosine or tryptophan). Compensatory “holes” of identical or similar size to the large side chain(s) are created on the interface of the second antibody molecule by replacing large amino acid side chains with smaller ones (e.g., alanine or threonine).

In some embodiments, the first Fc region comprises a first Fc mutation, and/or the second Fc region comprises a second Fc mutation, wherein: a) the first Fc mutations comprises T366W or S354C, and the second Fc mutation comprises Y349C, T366S, L368A, or Y407V; b) the first Fc mutation comprises D399K or E356K, and the second Fc mutation comprises K392D, or K409D; c) the first Fc mutation comprises E356K, E357K, or D399K, and the second Fc mutation comprises K370E, K409D, or K439E; d) the first Fc mutation comprises S364H, or F405A, and the second Fc mutation comprises Y349T, or T394F; e) the first Fc mutation comprises S364H, or T394F, and the second Fc mutation comprises Y394T, or F405A; f) the first Fc mutation comprises K370D, or K409D, and the second Fc mutation comprises E357K, or D399K; or g) the first Fc mutation comprises L351D, or L368E, and the second Fc mutation comprises L351K, or T366K, wherein numbering is according to the EU index.

In some embodiments, the first Fc region comprises a first Fc mutation, and/or the second Fc region comprises a second Fc mutation, wherein: a) the first Fc mutations comprises T366S/L368A/Y407V, and the second Fc mutation comprises T366W; b) the first Fc mutations comprises S354C/T366W, and the second Fc mutation comprises Y349C/T366S/L368A/Y407V; c) the first Fc mutations comprises T366Y, and the second Fc mutation comprises Y407T; d) the first Fc mutations comprises T366W, and the second Fc mutation comprises Y407A; e) the first Fc mutations comprises T394W, and the second Fc mutation comprises F405A; f) the first Fc mutations comprises T366Y/F405A, and the second Fc mutation comprises T394W/Y407T; g) the first Fe mutations comprises T366W/F405W, and the second Fe mutation comprises T394S/Y407A; g) the first Fe mutations comprises F405W, and the second Fe mutation comprises T394S; h) the first Fe mutations comprises D399C, and the second Fe mutation comprises K392C; i) the first Fc mutations comprises T366W/D399C, and the second Fe mutation comprises T366S/L368A/K392C/Y407V; j) the first Fe mutations comprises T366W/K392C, and the second Fe mutation comprises T366S/L368A/D399C/Y407V; k) the first Fe mutations comprises S354C/T366W, and the second Fe mutation comprises Y349C/T366S/L368A/Y407V; 1) the first Fe mutations comprises Y349C/T366W, and the second Fe mutation comprises S354C/T366S/L368A/Y407V; m) the first Fe mutations comprises E356C/T366W, and the second Fe mutation comprises Y349C/T366S/L368A/Y407V; n) the first Fe mutations comprises Y349C/T366W, and the second Fe mutation comprises E356C/T366S/L368A/Y407V; o) the first Fe mutations comprises E357C/T366W, and the second Fe mutation comprises Y349C/T366S/L368A/Y407V; p) the first Fe mutations comprises Y349C/T366W, and the second Fe mutation comprises E357C/T366S/L368A/Y407V.

In some embodiments, the polypeptide complex disclosed herein comprises a first CH3 region and a second CH3 region, wherein the first CH3 region or the second CH3 region comprises an amino acid sequence differing from wild-type IgG amino acid sequence such that one or more positive-charged amino acids (e.g., lysine, histidine and arginine) in the wild-type human IgG amino acid sequence are replaced with one or more negative-charged amino acids (e.g., aspartic acid and glutamic acid) at the corresponding position(s) in the CH3 region. Alternatively, the first CH3 region or the second CH3 region comprises van amino acid sequence differing from wild-type IgG amino acid sequence such that one or more negative-charged amino acids in the wild-type human IgG amino acid sequence are replaced with one or more positive-charged amino acids at the corresponding position(s) in the CH3 region.

In some embodiments, the first Fc region comprises a first Fc mutation, and/or the second Fe region comprises a second Fe mutation, wherein: a) the first Fc mutations comprises K370E/D399K/K439D, and the second Fc mutation comprises D356K/E357K/K409D; b) the first Fc mutations comprises K409D, and the second Fe mutation comprises D399K; c) the first Fe mutations comprises K409E, and the second Fe mutation comprises D399K; d) the first Fe mutations comprises K409E, and the second Fe mutation comprises D399R; e) the first Fe mutations comprises K409D, and the second Fe mutation comprises D399R; f) the first Fe mutations comprises D339K, and the second Fc mutation comprises E356K; g) the first Fc mutations comprises E356K/D399K, and the second Fe mutation comprises K392D/K409D; h) the first Fe mutations comprises E356K/D399K, and the second Fe mutation comprises K409D/K439D; i) the first Fe mutations comprises E357K/D399K, and the second Fe mutation comprises K370D/K409D; j) the first Fe mutations comprises E356K/E357K/D399K, and the second Fc mutation comprises K370D/K392D/K409D; k) the first Fe mutations comprises E357K/D399K, and the second Fe mutation comprises K392D/K409D; 1) the first Fe mutations comprises K392D/K409D, and the second Fe mutation comprises D399K; m) the first Fe mutations comprises K360D/K409D, and the second Fe mutation comprises D399K.

### V. Targets of the Polypeptide Complex

In some embodiments, in the polypeptide complex disclosed herein, at least one of the first target binding domain and the second target binding domain is chimeric, humanized, or fully human.

In some embodiments, the “chimeric target binding domain” herein means a recombinant protein that has the variable domains including the complementarity determining regions (CDRs) of an antibody derived from one species, such as a rodent antibody or a murine antibody, while the constant domains of the antibody molecule are derived from those of an antibody of a different species such as human antibody. For veterinary applications, the constant domains of the chimeric antibody may be derived from that of other species, such as a subhuman primate, eat or dog.

In some embodiments, the “humanized target binding domain” herein means a recombinant protein in which the CDRs from an antibody from one species, e.g., a rodent antibody, are transferred into framework regions of a human heavy and light variable domains. The constant domains of the antibody molecule are derived from those of a human antibody. In some embodiments, specific residues of the framework region of the humanized antibody, particularly those that are touching or close to the CDR sequences, may be modified, for example replaced with the corresponding residues from the original rodent, subhuman primate, or other antibody.

In some embodiments, at least one of the first target binding domain and the second target binding domain is fully human, which can be generated using any suitable methods known in the art, for example, from transgenic mice that have been genetically engineered to produce specific human antibodies in response to antigenic challenge. Methods for obtaining human antibodies from transgenic mice are described by Green et al, Nature Genet. 7: 13(1994), Lonberg et al, Nature 368: 856 (1994), and Taylor et al, Int. Immun. 6: 579 (1994). A fully human target binding domain also can be constructed by genetic or chromosomal transfection methods, as well as phage display technology, all of which are known in the art. See for example, McCafferty et al, Nature 348: 552-553 (1990) for the production of human antibodies and fragments thereof in vitro, from immunoglobulin variable domain gene repertoires from unimmunized donors. Phage display can be performed in a variety of formats, for their review, see e.g., Johnson and Chiswell, Current Opinion in Structural Biology 3: 5564-571 (1993). Human target binding domains may also be generated by in vitro activated B cells. See U.S. Pat. Nos. 5,567,610 and 5,229,275, which are incorporated herein by reference in their entirety.

The polypeptide complexes provided herein are based on novel 2:1 structure, and are bivalent for the second target and monovalent for the first target. Specifically, an additional Fab region is introduced on the basis of the IgG like bispecific antibody, so that one of the two target binding sites would be bivalent and regain its target binding affinity, which works effectively to adjust the affinity difference between two target binding sites without any adversely impact on the antibody specificity or any safety risks. Such affinity difference can be useful, because bivalent binding to the second target confers avidity and allows enhanced differentiation between cells with high and low expression of the second target. Moreover, the comparatively low affinity binding to the first target can be useful to reduce or avoid undesirable effects of strong binding to the first target (for example, non-specific activation of the first target which could lead to unwanted biological effects).

In some embodiments, the first target binding domain and the second target binding domain bind to different targets. In some embodiments, at least one of the first target and the second target is a disease-associated antigen. For example, a disease-associated antigen can be a tumor-associated antigen, or an antigen associated with an autoimmune diseases or an inflammatory disease, or an antigen associated with an eye disorder, an antigen associated with a central nervous system disease, an antigen associated with an infectious disease, or an antigen associated with a coagulation disorder.

In certain embodiments, one of the first target and the second target is a tumor-associated antigen. In some embodiments, the second target is tumor-associated antigen. In some embodiments, a tumor-associated antigen is an antigen that is present in a tumor that is not present in normal organs, tissues, and/or cells. In some embodiments, a tumor-associated antigen is an antigen that is more prevalent in a tumor than in normal organs, tissues, and/or cells. In some embodiments, a tumor-associated antigen is an antigen that is more prevalent in malignant cancer cells than in normal cells.

In some embodiments, the second target binding domain binds to a tumor-associated antigen. A tumor-associated antigen include antigens presented on a tumor cell surface, located on or within tumor cells, presented only by tumor cells and not by normal, i.e. non-tumor cells, representing a protein harboring one or more tumor-specific mutations compared to non-tumor cells, overexpressed in tumor cells when compared to non-tumor cells; accessible for antibody binding in tumor cells due to the less compact structure of the tumor tissue compared to non-tumor tissue, and presented on the vasculature of a tumor, etc. is meant an antigenic substance produced in tumor cells, i.e., it triggers an immune response in the host.

Examples of tumor-associated antigen include, but are not limited to: CD19, CD20, CD38, CD30, Her2/neu/ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin, etc.

In certain embodiments, one of the first target and the second target is a tumor-associated antigen, and the other is an immune related target. In some embodiments, the first target binding domain binds to an immune-related target. In some embodiments, the second target is tumor-associated antigen, and the first target binding domain binds to an immune-related target.

The immune related target provided herein may be selected from the group consisting of CD2, CD3, CD7, CD16, CD27, CD30, CD70, CD83, CD28, CD80 (B7-1), CD86 (B7-2), CD40, CD40L (CD154), CD47, CD122, CD137, CD137L, OX40 (CD134), OX40L (CD252), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), BTLA (CD272), BTLA, CD160, CTLA-4 (CD152), IDO1, IDO2, TDO, KIR, LAIR-1, NOX2, PD-1, PD-L1, PD-L2, TIM-3, VISTA, SIGLEC-7 (CD328), TIGIT, PVR (CD155), TGFβ, SIGLEC9 (CD329), and any combination thereof.

In some preferred embodiments of the bispecific polypeptide complexes provided herein, the second target relates to a receptor on the cytotoxic T lymphocytes (e.g. CD3), and the first target relates to a cell surface tumor antigen such as CD19, CD20, CD33, CD123, HER1, HER2, CEA, disialoganglioside GD2, PSMA, gpA33, EpCAM, P-cadherin, or B7H3 (Sedykh S E, et al., Drug Des Devel Ther. 2018; 12: 195-208.). As such, the bispecific polypeptide complexes provided herein can be used to form a link between T cells and tumor cells, which can cause the T cells to exert cytotoxic activity on tumor cells.

In certain embodiments, at least one of the first target and the second target is an antigen associated with an autoimmune disease or an inflammatory disease, such as, for example, rheumatoid arthritis (RA), psoriasis, osteoporosis, idiopathic pulmonary fibrosis, asthma, Sjogren syndrome, Type II diabetes. In some embodiments, the antigen associated with an autoimmune disease or an inflammatory disease is, for example but not limited to, HSA, TNF, IL6R, IL17A/F, RANKL, IL-13, IL4-IL17, BAFF, ICOSL, IL-17A, NGF, CD32b, CD79b, FGFR1, KLB, AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), myostatin, OX-40, scleroscin, SOST, TGF beta 1, TNF-α, or VEGF-A.

In certain embodiments, at least one of the first target and the second target is an antigen associated with an eye disease, such as, for example, age-related macular degeneration (AMD) and diabetic macular edema. In some embodiments, the antigen associated with an eye disease is, for example but not limited to, VEGF or ANG-2.

In certain embodiments, at least one of the first target and the second target is an antigen associated with a central nerves system disease, such as, for example, neuroblastoma, glioblastoma, Alzheimer Disease. In some embodiments, the antigen associated with an eye disease is, for example but not limited to, GD2, EGFRvIII, Aβ40, or Aβ42.

In certain embodiments, at least one of the first target and the second target is an antigen associated with an infectious disease, such as, for example, pneumonia, virus infection (such as COVID-19 infection). In some embodiments, the antigen associated with an autoimmune disease or an inflammatory disease is, for example but not limited to, Psl, Pcrv, or Spike protein of COVID-19 virus.

In certain embodiments, at least one of the first target and the second target is an antigen associated with a coagulation disorder, such as, for example, hemophilia A. In some embodiments, the antigen associated with hemophilia A is, for example but not limited to, FIXa, or FX.

Other examples of antigen pairs that can be targeted by the bispecific polypeptide complexes provided herein to thereby have potential therapeutic effects can include, but are not limited to, PD-L1:TGFβ, CD38:EGFR, HER2:VEGF, HER2:EGFR, PD-1:CTLA-4, PD-1:TIM3, OX40:PD-L1, FIXa:FX, CD32B:CD79B, Angiopoietin 2:VEGF, IL13:IL4, TNF:IL17A, DLL4:VEGF, IL1α:IL1β, FAP:DR5, CD30:gpA33, TNF:HSA, IL6R:HSA, IL17A/F:HSA, RANKL:HSA, Aβ40:Aβ42, IL13:IL17, FGFR1:KLB, PsI:PcrV, BAFF:B7RP1, NGF:TNF, and TNF:IL17A (Sedykh S E, et al., Drug Des Devel Ther. 2018; 12: 195-208).

In certain embodiments, the first target is CD3 and the second target is CD20.

In certain embodiments, the first target binding domain is capable of binding to CD3, and comprises a heavy chain CDR1 comprising the amino acid sequence of SEQ ID NO: 113, a heavy chain CDR2 comprising the amino acid sequence of SEQ ID NO: 114, a heavy chain CDR3 comprising the amino acid sequence of SEQ ID NO: 115, a light chain CDR1 comprising the amino acid sequence of SEQ ID NO: 116, a light chain CDR2 comprising the amino acid sequence of SEQ ID NO: 117, and a light chain CDR3 comprising the amino acid sequence of SEQ ID NO: 118.

In certain embodiments, the second target binding domain is capable of binding to CD20, and comprises a heavy chain CDR1 comprising the amino acid sequence of SEQ ID NO: 107, a heavy chain CDR2 comprising the amino acid sequence of SEQ ID NO: 108, a heavy chain CDR3 comprising the amino acid sequence of SEQ ID NO: 109, a light chain CDR1 comprising the amino acid sequence of SEQ ID NO: 110, a light chain CDR2 comprising the amino acid sequence of SEQ ID NO: 111, and a light chain CDR3 comprising the amino acid sequence of SEQ ID NO: 112.

### Exemplary Polypeptide Complexes

In certain embodiments of the polypeptide complex provided herein comprises: a) the fusion polypeptide comprises an amino acid sequence of Formula (I): VH2-CH1b-Linker-VL1-CLa, b) the second polypeptide comprises an amino acid sequence of Formula (II): VH1-CH1a, c) the third polypeptide comprises an amino acid sequence of Formula (III): VH2-CH1b, d) the fourth and fifth polypeptides each comprising an amino acid sequence of Formula (IV): VL2-CLb; wherein the pair of CH1b/CLb and/or the pair of CH1a/CLa are configured such that mispairing between CH1b and CLa and/or between CH1a and CLb is discouraged.

### Format New 1

In certain embodiments of the polypeptide complex provided herein, the CH1a has substitutions at heavy chain EU positions A141I, F170S, S181M, S183A, and V185A, and the CLa has substitutions at light chain EU positions F116A, A235V, S174A, S176F, and T178V; and the CH1b has substitution at heavy chain EU position S183E and the CLb has substitution at light chain EU position V133K. In certain embodiments, the CH1ₐ comprises the amino acid sequence of SEQ ID NO: 68, the CLₐ comprises the amino acid sequence of SEQ ID NO: 71, the CH1_{b} comprises the amino acid sequence of SEQ ID NO: 66, and the CL_{b} comprises the amino acid sequence of SEQ ID NO: 73.

In certain embodiments, the VH1 has substitution at heavy chain EU position Q39E, and the VL1 has substitution at light chain EU position Q38K. In certain embodiments, the VH2 has substitution at heavy chain EU position Q39K, and the VL1 has substitution at light chain EU position Q38E. In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 67, the VL1 comprises the amino acid sequence of SEQ ID NO: 70, the VH2 comprises the amino acid sequence of SEQ ID NO: 65, the VL2 comprises the amino acid sequence of SEQ ID NO: 72.

In certain of these embodiments, the first Fc and the second Fc comprises the amino acid sequences of SEQ ID NOs: 69 and 84. In certain of these embodiments, the fusion polypeptide, the second polypeptide, the third polypeptide, the fourth polypeptide comprise respectively the amino acid sequences of SEQ ID NOs: 6, 5, 7, and 8. This polypeptide complex is also referred to as Anti CD20×CD3 FORMAT NEW1.

### Format New 2

In certain embodiments of the polypeptide complex provided herein, the CH1a has substitution at EU position 126 for a cysteine residue, and substitution at EU position 220 for a non-cysteine residue; the CLa has substitution at EU position 121 for a cysteine residue, and substitution at EU position 214 for a non-cysteine residue. In certain of these embodiments, the CH1a comprises the amino acid sequence of SEQ ID NO: 77, the CLa comprises the amino acid sequence of SEQ ID NO: 79, the CH1b comprises the amino acid sequence of SEQ ID NO: 75, the CLb comprises the amino acid sequence of SEQ ID NO: 81.

In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 76, the VL1 comprises the amino acid sequence of SEQ ID NO: 78, the VH2 comprises the amino acid sequence of SEQ ID NO: 74, the VL2 comprises the amino acid sequence of SEQ ID NO: 80. In certain of these embodiments, the first Fc and the second Fc comprises the amino acid sequences of SEQ ID NOs: 69 and 84. In certain of these embodiments, the fusion polypeptide, the second polypeptide, the third polypeptide, the fourth polypeptide comprise respectively the amino acid sequences of SEQ ID NOs: 14, 13, 15, and 16. This polypeptide complex is also referred to as Anti CD20×CD3 FORMAT NEW2.

### Format New 3

In certain embodiments of the polypeptide complex provided herein, the CH1a has substitution at heavy chain EU position K147D, and the CLa has substitutions at light chain EU position S176K; and the CH1b has substitution at heavy chain EU position S183K and the CLb has substitution at light chain EU position S176D. In certain of these embodiments, the CH1ₐ comprises the amino acid sequence of SEQ ID NO: 88, the CLa comprises the amino acid sequence of SEQ ID NO: 90, the CH1b comprises the amino acid sequence of SEQ ID NO: 87, the CL_{b} comprises the amino acid sequence of SEQ ID NO: 92.

In certain embodiments, the VH1 has substitution at heavy chain EU positions Q39K and Q105K, and the VL1 has substitutions at light chain EU position Q38D and A43D. In certain embodiments, the VH2 has substitution at heavy chain EU positions Q39D and Q105D, and the VL2 has substitutions at light chain EU position Q38K and A43K. In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 85, the VL1 comprises the amino acid sequence of SEQ ID NO: 89, the VH2 comprises the amino acid sequence of SEQ ID NO: 86, the VL2 comprises the amino acid sequence of SEQ ID NO: 91. In certain of these embodiments, the first Fc and the second Fc comprises the amino acid sequences of SEQ ID NOs: 69 and 84. In certain of these embodiments, the fusion polypeptide, the second polypeptide, the third polypeptide, the fourth polypeptide comprise respectively the amino acid sequences of SEQ ID NOs: 22, 21, 23, and 24. This polypeptide complex is also referred to as Anti CD20×CD3 FORMAT NEW3.

### Format New 4

In certain embodiments of the polypeptide complex provided herein, the CH1a has substitution at heavy chain EU positions H168A and F170G, and the CLa has substitutions at light chain EU positions L135Y and S176W. In certain of these embodiments, the CH1a comprises the amino acid sequence of SEQ ID NO: 83, the CLₐ comprises the amino acid sequence of SEQ ID NO: 95, the CH1_{b} comprises the amino acid sequence of SEQ ID NO: 75, and the CL_{b} comprises the amino acid sequence of SEQ ID NO: 81.

In certain embodiments, the VH1 has substitutions at heavy chain EU positions 62E and Q39K, and the VL1 has substitutions at light chain EU position D1R and Q38D. In certain embodiments, the VH2 has substitution at heavy chain EU position Q39Y, and the VL2 has substitutions at light chain EU position Q38R. In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 93, the VL1 comprises the amino acid sequence of SEQ ID NO: 94, the VH2 comprises the amino acid sequence of SEQ ID NO: 82, the VL2 comprises the amino acid sequence of SEQ ID NO: 96. In certain of these embodiments, the first Fc and the second Fc comprises the amino acid sequences of SEQ ID NOs: 69 and 84. In certain of these embodiments, the fusion polypeptide, the second polypeptide, the third polypeptide, the fourth polypeptide comprise respectively the amino acid sequences of SEQ ID NOs: 30, 29, 31, and 32. This polypeptide complex is also referred to as Anti CD20×CD3 FORMAT NEW4.

### Format New 7

In certain embodiments of the polypeptide complex provided herein, the CH1a has substitution at EU position 173 for a cysteine residue, substitution at EU position 220 for a non-cysteine residue, and substitution at EU position S183K; the CLa has substitution at EU position 160 for a cysteine residue, substitution at EU position 214 for a non-cysteine residue, and substitution at EU position S176D. In certain of these embodiments, the CH1a comprises the amino acid sequence of SEQ ID NO: 103, the CLa comprises the amino acid sequence of SEQ ID NO: 104, the CH1b comprises the amino acid sequence of SEQ ID NO: 75, the CLb comprises the amino acid sequence of SEQ ID NO: 81.

In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 76, the VL1 comprises the amino acid sequence of SEQ ID NO: 78, the VH2 comprises the amino acid sequence of SEQ ID NO: 74, the VL2 comprises the amino acid sequence of SEQ ID NO: 80. In certain of these embodiments, the first Fc and the second Fc comprises the amino acid sequences of SEQ ID NO: 69 and 84. In certain of these embodiments, the fusion polypeptide, the second polypeptide, the third polypeptide, the fourth polypeptide comprise respectively the amino acid sequences of SEQ ID NOs: 54, 53, 55, and 56. This polypeptide complex is also referred to as Anti CD20×CD3 FORMAT NEW7.

### Format New 5

In certain embodiments of the polypeptide complex provided herein comprises: a) the fusion polypeptide comprises an amino acid sequence of Formula (I): VH2-CH1b-Linker-VL1-TCRβ, b) the second polypeptide comprises an amino acid sequence of Formula (II): VH1-TCRα, c) the third polypeptide comprises an amino acid sequence of Formula (III): VH2-CH1b, d) the fourth and fifth polypeptides each comprising an amino acid sequence of Formula (IV): VL2-CLb.

In certain of these embodiments, the TCRα comprises the amino acid sequence of SEQ ID NO: 97, the TCRβ comprises the amino acid sequence of SEQ ID NO: 98, the CH1b comprises the amino acid sequence of SEQ ID NO: 75, the CLb comprises the amino acid sequence of SEQ ID NO: 81.

In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 76, the VL1 comprises the amino acid sequence of SEQ ID NO: 78, the VH2 comprises the amino acid sequence of SEQ ID NO: 74, the VL2 comprises the amino acid sequence of SEQ ID NO: 80. In certain of these embodiments, the first Fc and the second Fc comprises the amino acid sequences of SEQ ID NOs: 69 and 84. In certain of these embodiments, the fusion polypeptide, the second polypeptide, the third polypeptide, the fourth polypeptide comprise respectively the amino acid sequences of SEQ ID NOs: 38, 37, 39, and 40. This polypeptide complex is also referred to as Anti CD20×CD3 FORMAT NEW5.

### Format New 6

In certain embodiments of the polypeptide complex provided herein comprises: a) the fusion polypeptide comprises an amino acid sequence of Formula (I): VH2-CH1b-Linker-VH1-CLₐ, b) the second polypeptide comprises an amino acid sequence of Formula (II): VL1-CH1ₐ, c) the third polypeptide comprises an amino acid sequence of Formula (III): VH2-CH1_{b}, d) the fourth and fifth polypeptides each comprising an amino acid sequence of Formula (IV): VL2-CL_{b}.

In certain embodiments of the polypeptide complex provided herein, the CH1ₐ comprises the amino acid sequence of SEQ ID NO: 100, the CLₐ comprises the amino acid sequence of SEQ ID NO: 101, the CH1_{b} comprises the amino acid sequence of SEQ ID NO: 99, the CL_{b} comprises the amino acid sequence of SEQ ID NO: 102.

In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 76, the VL1 comprises the amino acid sequence of SEQ ID NO: 78, the VH2 comprises the amino acid sequence of SEQ ID NO: 74, the VL2 comprises the amino acid sequence of SEQ ID NO: 80. In certain of these embodiments, the first Fc and the second Fc comprises the amino acid sequences of SEQ ID NO: 69 and 84. In certain of these embodiments, the fusion polypeptide, the second polypeptide, the third polypeptide, the fourth polypeptide comprise respectively the amino acid sequences of SEQ ID NOs: 46, 45, 47, and 48. This polypeptide complex is also referred to as Anti CD20×CD3 FORMAT NEW6.

### Format New 8

In certain embodiments, the polypeptide complex provided herein comprises: a) the fusion polypeptide comprises an amino acid sequence of Formula (I): VH2-CH1b-Linker-VL1-CH1a, b) the second polypeptide comprises an amino acid sequence of Formula (II): VH1-CLa, c) the third polypeptide comprises an amino acid sequence of Formula (III): VH2-CH1b, d) the fourth and fifth polypeptides each comprising an amino acid sequence of Formula (IV): VL2-CLb.

In certain embodiments of the polypeptide complex provided herein, the CH1a comprises the amino acid sequence of SEQ ID NO: 105, the CLa comprises the amino acid sequence of SEQ ID NO: 106, the CH1_{b} comprises the amino acid sequence of SEQ ID NO: 75, the CLb comprises the amino acid sequence of SEQ ID NO: 81.

In certain embodiments, the VH1 comprises a heavy chain CDR1/CDR2/CDR3 comprising the amino acid sequences of SEQ ID NO: 113, SEQ ID NO: 114 and SEQ ID NO: 115, respectively, and/or the VL1 comprises a light chain CDR1/CDR2/CDR3 comprising the amino acid sequences of SEQ ID NO: 116, SEQ ID NO: 117, and SEQ ID NO: 118, respectively. In certain embodiments, the VH2 comprises a heavy chain CDR1/CDR2/CDR3 comprising the amino acid sequences of SEQ ID NO: 107, SEQ ID NO: 108 and SEQ ID NO: 109, respectively, and/or the VL2 comprises a light chain CDR1/CDR2/CDR3 comprising the amino acid sequences of SEQ ID NO: 110, SEQ ID NO: 111, and SEQ ID NO: 112, respectively.

In certain embodiments of the polypeptide complex provided herein, the VH1 comprises the amino acid sequence of SEQ ID NO: 76, the VL1 comprises the amino acid sequence of SEQ ID NO: 78, the VH2 comprises the amino acid sequence of SEQ ID NO: 74, the VL2 comprises the amino acid sequence of SEQ ID NO: 80. In certain of these embodiments, the first Fc and the second Fc comprises the amino acid sequences of SEQ ID NO: 69 and 84. In certain of these embodiments, the fusion polypeptide, the second polypeptide, the third polypeptide, the fourth polypeptide comprise respectively the amino acid sequences of SEQ ID NOs: 62, 61, 63, and 64. This polypeptide complex is also referred to as Anti CD20×CD3 FORMAT NEW8.

### VI. Polynucleotides and Recombinant Methods

The present disclosure provides a nucleic acid comprising a nucleotide sequence that encodes the fusion polypeptide or the polypeptide complex provided herein. The term “nucleic acid” or “nucleotide sequence” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The polynucleotides encoding the polypeptide complex disclosed herein may be generated using methods known in the art. In certain embodiments, the sequence of the polynucleotides may be obtained based on the amino acid sequences of the polypeptide complex, and nucleic acids can be generated using synthetic methods. Alternatively, the polynucleotides provided herein can also be obtained from another available nucleic acid that encodes a polypeptide with a sequence homologous to the polypeptides in the polypeptide complex disclosed herein. Then a DNA manipulation process can be applied to manipulate the sequence of the parent antibody-encoding nucleic acid, such as introducing mutations, insertion, deletion, etc., so as to obtain the nucleic acid encoding the polypeptide complex disclosed herein.

The nucleotide sequence that encodes the fusion polypeptide or the polypeptide complex can be inserted into one or more vector(s) for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), a transcription termination sequence, and one or more other regulatory elements.

The present disclosure provides vectors comprising the nucleic acid provided herein. In certain embodiments, the nucleic acid provided herein encodes the antibodies, with at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the nucleotide sequence encoding the fusion polypeptide or the polypeptide complex can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenformis, Pseudomonas such as P. aeruginosa, and Streptomyces.

In addition to prokaryotes, eukaryotic microbes such as filamentous fungi or yeast are suitable cloning or expression hosts for the polypeptide complex-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated polypeptide complex provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa calfornica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for antibody production and cultured in conventional nutrient media modified as appropriate for inducing promoters, selecting transformants, or amplifying the genes encoding the desired sequences. In another embodiment, the antibodies may be produced by homologous recombination known in the art. In certain embodiments, the host cell is capable of producing the fusion polypeptide or the polypeptide complex provided herein.

The present disclosure also provides a method of expressing the fusion polypeptide or the polypeptide complex provided herein, comprising culturing the host cell provided herein under the condition at which the vector of the present disclosure is expressed. The host cells used to produce the antibodies provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the fusion polypeptide or the polypeptide complex can be produced intracellularly, in the periplasmic space, or directly secreted into the medium. If the fusion polypeptide or the polypeptide complex is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the fusion polypeptide or the polypeptide complex is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The fusion polypeptide or the polypeptide complex prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the fusion polypeptide or the polypeptide complex. The suitability of protein A as an affinity ligand depends on the species and isotype of any immunoglobulin Fc domain that is present in the polypeptide complex. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the polypeptide complex comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the fusion polypeptide or the polypeptide complex of interest and contaminants may be subjected to low pH hydrophobic interaction chromatography using an elution buffer at a pH between about 2.5-4.5, preferably performed at low salt concentrations (e.g., from about 0-0.25M salt).

### VI. Pharmaceutical Composition

The present invention further provides a pharmaceutical composition comprising the polypeptide complex described herein and a pharmaceutically acceptable carrier.

As used herein, the term “pharmaceutically acceptable” indicates that the designated carrier, vehicle, diluent, excipient(s), salt and/or medium is generally chemically and/or physiologically compatible with other ingredients, such as the active ingredient (i.e. the polypeptide complex or the heterodimeric antibody or antigen-binding fragment thereof) comprising the formulation, and is physiologically compatible with a subject receiving the pharmaceutical composition.

A “pharmaceutically acceptable carrier” refers to an ingredient in a pharmaceutical formulation, other than an active ingredient, which is bioactivity acceptable and nontoxic to a subject. In the context of the present disclosure, a pharmaceutical acceptable carrier for use in the pharmaceutical composition disclosed herein may include, for example, pharmaceutically acceptable liquid, gel or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Herein, suitable “components” may include, for example, antioxidants, fillers, binders, disintegrants, buffers, preservatives, lubricants, flavorings, thickeners, coloring agents, emulsifiers or stabilizers such as sugars and cyclodextrins. Suitable “antioxidants” may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a pharmaceutical composition provided herein decreases oxidation of the polypeptide complex or heterodimeric antibody or antigen-binding fragment thereof. This reduction in oxidation prevents or reduces loss of binding affinity, thereby improving protein stability and maximizing shelf-life. Therefore, in certain embodiments, a pharmaceutical composition is provided that comprise, in addition to the active ingredient (i.e. the polypeptide complex or the heterodimeric antibody or antigen-binding fragment thereof disclosed herein), one or more antioxidants such as methionine.

The pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Pharmaceutically acceptable “diluents” may include saline and aqueous buffer solutions.

Pharmaceutically acceptable “adjuvants” may include preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving the polypeptide complex as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the polypeptide complex, the polypeptide complex. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

In certain embodiments, a composition is further provided, comprising a pharmaceutically acceptable carrier, diluent or adjuvant, and an active ingredient. The active ingredient can be the polypeptide complex disclosed herein, or a conjugate of the polypeptide complex disclosed herein.

### VIII. Conjugates

The polypeptide complex as provided herein can be used in a non-conjugated form or in a conjugated form.

In a conjugated form, the polypeptide complexes are conjugated to one or more desired conjugate moieties, i.e. heterologous moieties, to realize certain functionalities, e.g. to facilitate target detection or for imaging or therapy.

Herein, the present disclosure provides a conjugate, which comprises the polypeptide complex provided herein, and a conjugate moiety (e.g. payload) that is conjugated thereto. The payload can be any one of the group consisting of a radioactive label, a fluorescent label, an enzyme-substrate label, an affinity purification tag, a tracer molecule, an anticancer drug, and a cytotoxic molecule.

A variety of conjugates can be linked to the engineered antibody provided herein by covalent binding, affinity binding, intercalation, coordinate binding, complexation, association, blending, or addition, among others.(see, e.g., “Conjugate Vaccines”, Contributions to Microbiology and Immunology, J. M. Cruse and R. E. Lewis, Jr. (eds.), Carger Press, New York, (1989)).

In certain embodiments, the polypeptide complex provided herein may be engineered to contain specific sites outside the epitope binding portion that may be specifically utilized for binding to one or more conjugates. For example, such a site may include one or more reactive amino acid residues, such as for example cysteine or histidine residues, to facilitate covalent linkage to a conjugate.

In certain embodiments, the N-terminus and/or C-terminus of the polypeptide complex provided herein can also serve to provide reactive groups for conjugation. For example, the N-terminus can be conjugated to one moiety (e.g. polyethylene glycol (PEG), etc.) and the C-terminus is conjugated to another moiety (e.g. biotin, etc.).

In certain embodiments, the polypeptide complex provided herein may be linked to a conjugate directly, or indirectly for example through another conjugate or through a linker.

For example, the polypeptide complex provided herein having a reactive residue such as cysteine may be linked to a thiol-reactive agent in which the reactive group is, for example, a maleimide, an iodoacetamide, a pyridyl disulfide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

For another example, the polypeptide complex provided herein may be conjugated to biotin, then indirectly conjugated to a second conjugate that is conjugated to avidin. For still another example, the polypeptide complex may be linked to a linker which further links to the conjugate. Examples of linkers include bifunctional coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suherate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and his-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 (1978)) and N-succinimidyl-4-(2-pyridylthio) pentanoate (SPP) to provide for a disulfide linkage.

In certain embodiments, the conjugate moiety comprises an agent for detection or isolation, such as a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, or other anticancer drugs.

The conjugate moiety can be a detectable label, a pharmacokinetic modifying moiety, a purification moiety, a cytotoxic moiety or a therapeutic agent. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels), chromophoric moiety, digoxigenin, biotin/avidin, a DNA molecule or gold for detection.

In certain embodiments, the conjugate moiety can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. The polymer may be of any molecular weight and may be branched or unbranched. The number of polymers attached to the antibody may vary, and if more than one polymer is attached, they can be the same or different molecules. In certain embodiments, the conjugate can be a purification moiety such as a magnetic bead.

In certain embodiments, the conjugate moiety can be a cytotoxic moiety. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In some embodiments, the conjugate moiety comprises an enzymatically active toxin or a fragment thereof, including but not limited to diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes.

Methods for the conjugation of conjugate moieties to proteins such as antibodies, immunoglobulins or fragments thereof are found, for example, in U.S. Pat. Nos. 5,208,020; 6,441,163; WO2005037992; WO2005081711; and WO2006/034488, which are incorporated herein by reference to the entirety.

In certain embodiments, the polypeptide complex provided herein are used as a base for a conjugate.

### IX. Composition

In another aspect, the present invention provides a composition comprising the polypeptide complex or the conjugate described herein and a pharmaceutically acceptable carrier.

### X. Medical Use

In another aspect, the present invention provides a method for treating a disease condition in a subject that is in need of such treatment, comprising: administering to the subject a therapeutically effective amount of the polypeptide complex of the present invention, the pharmaceutical composition described herein, the conjugate described herein, or the composition described herein.

As used herein, the term “subject” or “individual” or “animal” or “patient” refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on. In certain embodiments, the subject is human.

As used herein, “treatment” of a condition may include, alleviating a condition, slowing the onset or rate of development of a condition, delaying the development of symptoms associated with a condition, reducing or ending symptoms associated with a condition, generating a complete or partial regression of a condition, or some combinations thereof.

As used herein, the term “disorder,” “disease,” “condition” or alike, refers to a condition that affects a subject who would nonetheless benefits from treatment with the polypeptide complex.

As used herein, the term “therapeutically effective amount” of a therapeutic agent refers to an amount of the therapeutic agent that, when taken by a subject in an appropriate manner, can generate sufficient therapeutic effects to the subject. It is to be understood that just like other therapeutic drugs, the therapeutically effective amount of the polypeptide complex as provided above will be influenced by various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the polypeptide complex as provided herein may be administered at a therapeutically effective amount of about 0.01 mg/kg to about 100 mg/kg. Dosage regimens may be adjusted to provide the optimum desired response (e.g., a therapeutic response). For example, a single dose may be administered, or several divided doses may be administered over time.

The polypeptide complex described above and the method disclosed herein can be applied to treat a wide variety of diseases. In humans, and other primates as well, the diseases that are contemplated to be treatable by the polypeptide complex described above and the method disclosed herein can include the following:

(1) cancers and other hyperproliferative disorders, including both benign or malignant tumors, leukemia and lymphoid malignancies. Depending on the cell type having cancers or hyperproliferative disorders, examples include neuronal, glial, astrocytal, hypothalamic, glandular, macrophagal, epithelial, endothelial, and stromal malignancies. Depending on the organ/location afflicted with cancers or hyperproliferative disorders, examples include: cancers of the head, neck, eye, mouth, throat, esophagus, chest, skin, bone, lung, colon, rectum, colorectal, stomach, spleen, kidney, skeletal muscle, subcutaneous tissue, metastatic melanoma, endometrial, prostate, breast, ovaries, testicles, thyroid, blood, lymph nodes, kidney, liver, pancreas, brain, or central nervous system;

(2) autoimmune and/or inflammatory disorders, including alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, Sjogren's syndrome, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation, sepsis, rheumatoid arthritis, peritonitis, Crohn's disease, reperfusion injury, septicemia, endotoxic shock, cystic fibrosis, endocarditis, psoriasis, arthritis (e.g., psoriatic arthritis), anaphylactic shock, organ ischemia, reperfusion injury, spinal cord injury and allograft rejection, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitisherpetiformis vasculitis, vitiligo, and Wegener's granulomatosis. Inflammatory disorders, can further include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacterial infections;

(3) infectious and parasitic diseases, such as those caused by viruses (e.g. HBV, HCV, HIV, RSV, hMPV, PIV, coronaviruses, or influenza viruses, etc.), fungi (e.g. Naegleria, Aspergillus, Blastomyces, Histoplasma, Candida or Tinea genera, etc.), eukaryotic microbes (e.g. Giardia, Toxoplasma, Plasmodium, Trypanosoma, and Entamoeba genera, etc.), and bacteria (Staphylococcus, Streptococcus, Pseudomonas, Clostridium, Borrelia, Vibro and Neiserria genera, etc.);

(4) other disease or disorders, including those not covered by an of the above in (1)-(3), such as cardiovascular diseases, neuropathies, neuropsychiatric conditions, injuries, or coagulation disorder, etc.

In some embodiments, the disease is selected from the group consisting of a cancer, an inflammatory disease, an infectious or parasitic disease, a cardiovascular disease, eye disease, central nerves system (CNS) disease, an injury, metabolic disease, autoimmune disease, or a coagulation disorder. In some embodiments, the CNS disease is neuropathy, a neuropsychiatric condition, neuroblastoma, glioblastoma, or Alzheimer Disease.

The polypeptide complex disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the polypeptide complex disclosed herein may be administered alone or in combination with one or more additional therapeutic means or agents. For example, the polypeptide complex disclosed herein may be administered in combination with another therapeutic agent, for example, a chemotherapeutic agent or an anti-cancer drug.

In certain of these embodiments, the polypeptide complex as disclosed herein that is administered in combination with one or more additional therapeutic agents may be administered simultaneously with the one or more additional therapeutic agents, and in certain of these embodiments the polypeptide complex and the additional therapeutic agent(s) may be administered as part of the same pharmaceutical composition. However, the polypeptide complex administered “in combination” with another therapeutic agent does not have to be administered simultaneously with or in the same composition as the agent. The polypeptide complex administered prior to or after another agent is considered to be administered “in combination” with that agent as the phrase is used herein, even if the polypeptide complex and second agent are administered via different routes. Where possible, additional therapeutic agents administered in combination with the polypeptide complex disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

### XI. Methods of Antigen Detection

In another aspect, the present disclosure provides a method of detecting presence or amount of an antigen in a sample. In some embodiments, the method comprises contacting a sample suspected of containing the antigen with the polypeptide complex described herein and determining the formation of a complex between the antigen and the polypeptide complex. In some embodiments, the antigen is a disease-associated antigen, for example, a tumor-associated antigen. In certain embodiments, the polypeptide complex disclosed herein is used in a method of diagnosing a subject suffering from a disease (e.g. cancer), the method comprising: determining the presence or amount of the disease-related (e.g. tumor-related) antigen in a sample obtained from the subject by contacting the sample with a polypeptide complex of the disclosure and detecting the presence of the antigen bound polypeptide complex.

Any sample suspected of containing the tumor-associated antigen can be used, such as a biological fluid such as a blood sample, a plasma sample, a urine sample, and the like, as well as biopsy of a disease tissue (e.g. tumor tissue).

The presence or level of the disease-associated (e.g. tumor-associated) antigen in a sample can be determined based on the detection of the presence or level of the complex of the disease-associated (e.g. tumor-associated) antigen bound by the polypeptide complex or the antigen binding fragment thereof disclosed herein. Any suitable methods can be used for such detection, for example, by immunoassays such as immunohistochemistry (IHC), immunofluorescence (IF), immunoblotting (e.g., Western blotting), flow cytometry (e.g., 15 FACS™), Enzyme-linked Immunosorbent Assay (ELISA), enzyme immunoassay (EIA), and radioimmunoassay (RIA).

For a review of immunological and immunoassay procedures, see Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7th ed. 1991).

In certain embodiments, the polypeptide complex disclosed herein are detectably labeled by a conjugated payload, or are not labeled but can react with a second molecule which is detectably labeled (e.g. a detectably labeled secondary antibody).

In certain embodiments, the polypeptide complex disclosed herein may be immobilized on a solid substrate. The immobilization can be via covalent linking or non-covalent attachment (e.g. coating). Examples of solid substrate include porous and non-porous materials, latex particles, magnetic particles, microparticles, strips, beads, membranes, microtiter wells and plastic tubes. The choice of solid phase material and method of detectably labeling can be determined based upon desired assay format performance characteristics.

The level of an antigen can be determined, for example, by normalizing to a control value or to a standard curve. The control value can be predetermined or determined concurrently.

The assays and methods provided herein for the measurement of the level of an antigen can be adapted or optimized for use in automated and semi-automated systems or point of care assay systems.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### 1.1 Antibody Construction

Bispecific antibodies were constructed using both the new 2:1 format disclosed in the present invention (i.e. designated as FORMAT NEW below, and shown in FIG. 5A), and the existing 2:1 format known in the art (i.e. designated as FORMAT EX below, and shown in FIG. 4A). To demonstrate the advantages of FORMAT NEW over the FORMAT EX, 8 pairs of bispecific antibodies were constructed and expressed, which are otherwise identical except for using the FORMAT EX or using the FORMAT NEW, so as to allow head-to-head comparison of the two formats.

Both the FORMAT NEW and FORMAT EX are IgG like bispecific antibodies, which are composed mainly of three Fab domains derived from two monoclonal antibodies, in which one Fab domain is derived from the first antibody, and the other two Fab domains are derived from the second antibody (FIG. 4A and FIG. 5A). The first antibody heavy chain was named B1 (including VH1 and CH1a) and the first antibody light chain was named A1 (including VL1 and CLa), which is monovalent in the bispecific antibodies. The second antibody heavy chain was named B2 (including VH2 and CH1b) and the second antibody light chain was named A2 (including VL2+CLb), which are bivalent in the bispecific antibodies. The linker (GGGGS)₂ was used in the bispecific antibodies to link different functional domains.

Two monoclonal antibodies targeting CD20 and CD3, respectively, were selected to build the bispecific antibodies used in the examples. In the constructed illustrative bispecific antibodies, the anti-CD20 antibody were made to be bivalent (i.e. corresponding to A2 and B2 in the FIG. 4A and FIG. 5A), and the anti-CD3 was monovalent (i.e. corresponding to A1 and B1 in the FIG. 4A and FIG. 5A). The anti-CD20 antibody used in this example is 2F2, and has been previously reported in US20040167319. The anti-CD3 antibody used in this example is SP34, and has been previously reported in The EMBO Journal vol. 4 no. 2 pp. 337-344, 1985

To reduce mispairing, mutations or cross configurations were introduced in the Fab regions of the first and second antibodies, respectively, and the details are as shown in Table 1 below.

TABLE 1
Mutations or configurations introduced in the bispecific antibodies

| | First Fab | | | Second Fab | | |
|---|---|---|---|---|---|---|
| Format | | VH1 or | | | | CH1b |
| New | Fragment | VL1 | CH1a or CLa | Fragment | VH2 or VL2 | or CLb |
| 1 | B1 | Q39E | yt65H(A141I:F170S:S181M:S183A:V185A) | B2 | Q39K | S183E |
| | (VH1-CH1ₐ) | | | (VH2-CH1_{b}) | | |
| | A1 | Q38K | yt65L(F116A:L135V:S174A:S176F:T178V) | A2 | Q38E | V133K |
| | (VL1-CLₐ) | | | (VL2-CL_{b}) | | |
| 2 | B1 | | F126C, C220V | B2 | | |
| | (VH1-CH1ₐ) | | | (VH2-CH1_{b}) | | |
| | A1 | | S121C, C214V | A2 | | |
| | (VL1-CLₐ) | | | (VL2-CL_{b}) | | |
| 3 | B1 | Q39K, | K147D | B2 | Q39D, | S183K |
| | (VH1-CH1ₐ) | Q105K | | (VH2-CH1_{b}) | Q105D | |
| | A1 | Q38D, | S176K | A2 | Q38K, A43K | S176D |
| | (VL1-CLₐ) | A43D | | (VL2-CL_{b}) | | |
| 4 | B1 | 62E, | H168A, F170G | B2 | Q39Y | |
| | (VH1-CH1ₐ) | Q39K | | (VH2-CH1_{b}) | | |
| | A1 | DIR, | L135Y, S176W | A2 | Q38R | |
| | (VL1-CLₐ) | Q38D | | (VL2-CL_{b}) | | |
| 5 | B1 | | TCRα | B2 | | |
| | (VH1-TCRα) | | | (VH2-CH1_{b}) | | |
| | A1 | | TCRβ | A2 | | |
| | (VH1-TCRβ) | | | (VL2-CL_{b}) | | |
| 6 | B1 | VL1 | | B2 | VH2 | |
| | (VL1-CH1ₐ) | | | (VH2-CH1_{b}) | | |
| | A1 | VH1 | | A2 | VL2 | |
| | (VH1-CL1ₐ) | | | (VL2-CL_{b}) | | |
| 7 | B1 | | V173C, S183K, C220S | B2 | | |
| | (VH1-CH1ₐ) | | | (VH2-CH1_{b}) | | |
| | A1 | | Q160C, S176D, C214S | A2 | | |
| | (VL1-CLₐ) | | | (VL2-CL_{b}) | | |
| 8 | B1 | | CL | B2 | | CH1 |
| | (VH1-CLₐ) | | | (VH2-CH1_{b}) | | |
| | A1 | | CH1 | A2 | | CL |
| | (VL1-CH1ₐ) | | | (VL2-CL_{b}) | | |

Mutations were also introduced in CH3 regions to promote heterodimerization of the first Fc region and the second Fc region. Specifically, one of the CH3 regions introduced T366S, L368A, and Y407V, while the other one was introduced T366W mutation. The mutations of CH3 regions in the following examples provided for the knob-into-hole design in the bispecific antibodies.

Based on the bispecific antibody structure indicated above (Table 1), 8 pairs of bispecific antibodies were constructed and expressed by using the novel 2:1 format of the present invention: FORMAT NEW (as shown in FIG. 4A), and by using the conventional 2:1 format: FORMAT EX (as shown in FIG. 5A). These 8 pairs of bispecific antibodies (i.e. a total of 16 bispecific antibodies) were recombinantly expressed, and designated as FORMAT NEW1 to FORMAT NEW8, and FORMAT EX1 to FORMAT EX8, respectively. For each antibody, full length amino acid sequences for each of the polypeptide chains were set forth as follows, with functional domains marked using different underlines set forth in the Notes below the sequences.

FORMAT EX1
a) FORMAT EX1 Chain 2 (AH): CD20HC+CD3HC(Knob)
SEQ ID No.: 1
EVQLVESGGGLVQPGRSLRLSCAASGFTFNDYAMHWVRKAPGKGLEW
VSTISWNSGSIGYADSVKGRFTISRDNAKKSLYLQMNSLRAEDTALYYCA

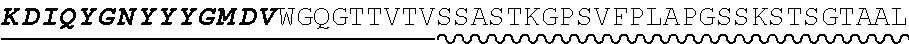

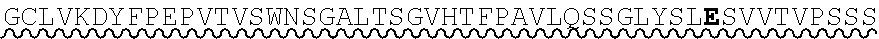

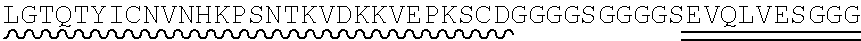

LVQPGGSLRLSCAASGFTESTYAMNWVREAPGKGLEWVGRIRSKYNNY
ATYYADSVKGRFTISRDDSKNSLYLQMNSLRAEDTAVYYCVRHGNEGNS
YVSWFAYWGQGTLVTVSSASTKGPSVFPLAPSSKSTSGGTAILGCLVKDY

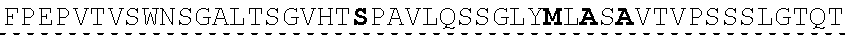

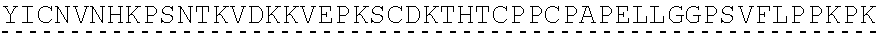

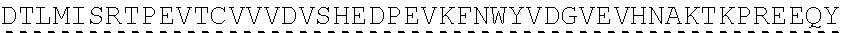

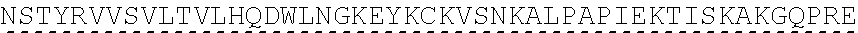

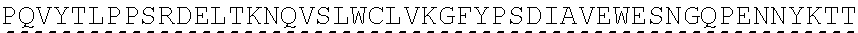

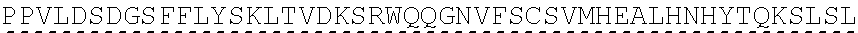

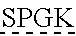

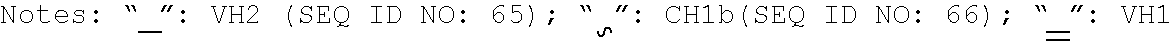

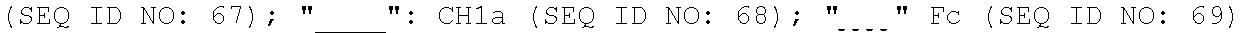

b) FORMAT EX1 Chain1 (AL): CD3 LC
SEQ ID No.: 2
QAVVTQEPSLTVSPGGTVTLTCRSSTGAVTTSNYANWVQKKPGQAPRGL
IGGTNKRAPWTPARFSGSLLGGKAALTLSGAQAEDEADYYCALWYSNLW

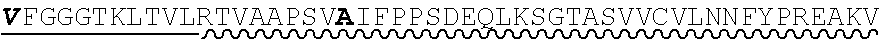

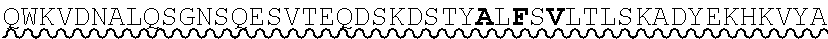

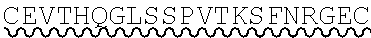

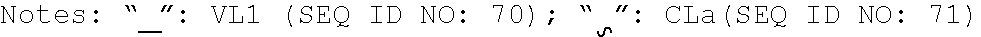

c) FORMAT EX1 Chain3 (BH): CD20 HC(Hole)
SEQ ID No.: 3
EVQLVESGGGLVQPGRSLRLSCAASGFTFNDYAMHWVRKAPGKGLEW
VSTISWNSGSIGYADSVKGRFTISRDNAKKSLYLQMNSLRAEDTALYYCA

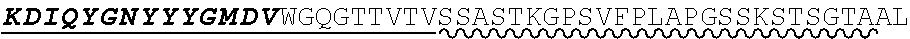

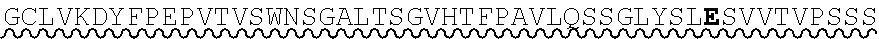

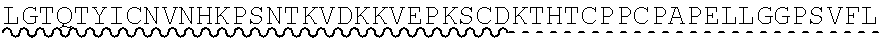

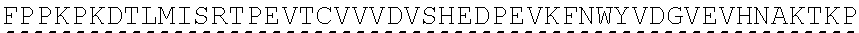

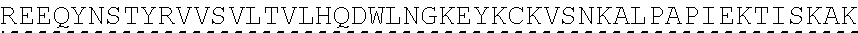

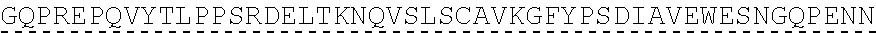

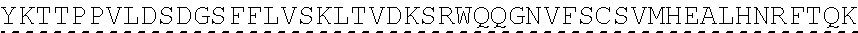

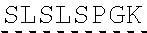

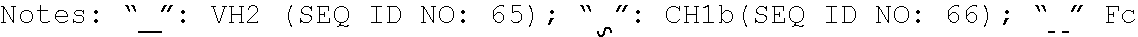

(SEQ ID NO: 84)
d) FORMAT EX1 Chain4 (BL): CD20 LC
SEQ ID No.: 4
EIVLTQSPATLSLSPGERATLSCRASQSVSSYLAWYQEKPGQAPRLLIYDAS
NRATGIPARFSGSGSGTDFTLTISSLEPEDFAVYYCQQRSNWPITFGQGTRL

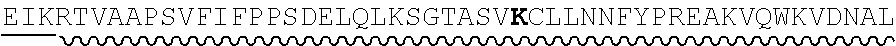

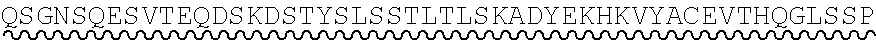

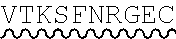

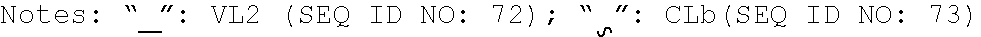

FORMAT NEW1
a) FORMAT NEW1 Chain2 (B1): anti-CD3 Heavy Chain (Knob)
SEQ ID No.: 5
EVQLVESGGGLVQPGGSLRLSCAASGFTFSTYAMNWVREAPGKGLEWV
GRIRSKYNNYATYYADSVKGRFTISRDDSKNSLYLQMNSLRAEDTAVYY

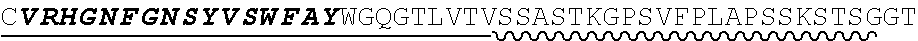

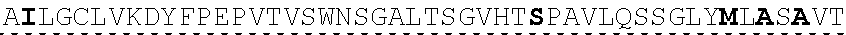

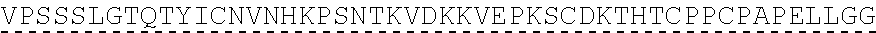

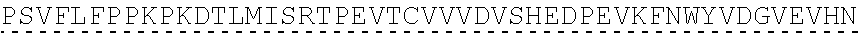

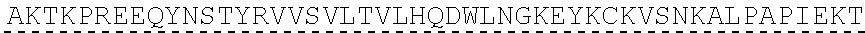

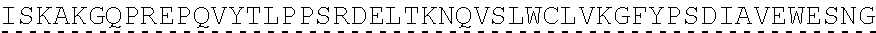

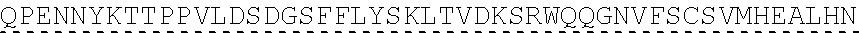

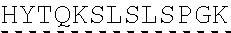

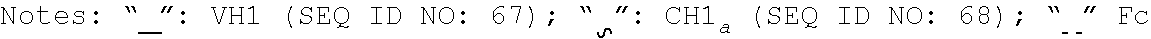

(SEQ ID NO: 69)
b) FORMAT NEW1 Chain1 (B2-A1): anti-CD20 Heavy Chain+anti-CD3 Light
Chain
SEQ ID No.: 6
EVQLVESGGGLVQPGRSLRLSCAASGFTFNDYAMHWVRKAPGKGLEW
VSTISWNSGSIGYADSVKGRFTISRDNAKKSLYLQMNSLRAEDTALYYCA

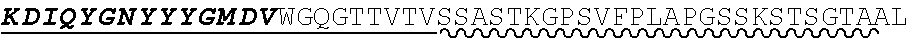

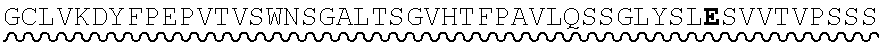

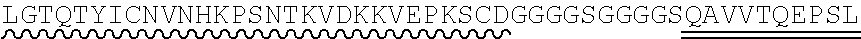

TVSPGGTVTLTCRSSTGAVTTSNYANWVQKKPGQAPRGLIGGTNKRAPW
TPARFSGSLLGGKAALTLSGAQAEDEADYYCALWYSNLWVFGGGTKLTV

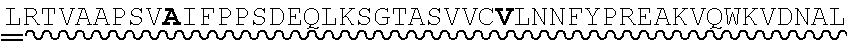

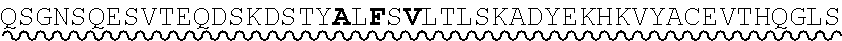

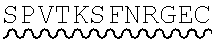

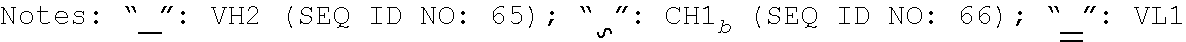

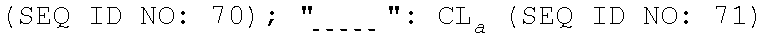

c) FORMAT NEW1 Chain3 (B2): anti-CD20 Heavy Chain (Hole)
SEQ ID No.: 7
EVQLVESGGGLVQPGRSLRLSCAASGFTFNDYAMHWVRKAPGKGLEW
VSTISWNSGSIGYADSVKGRFTISRDNAKKSLYLQMNSLRAEDTALYYCA

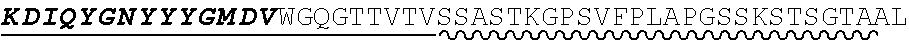

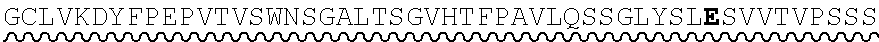

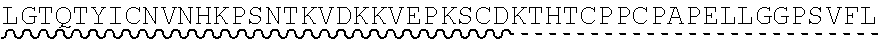

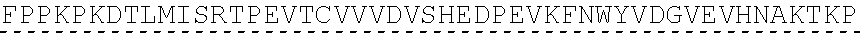

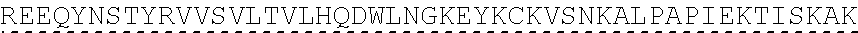

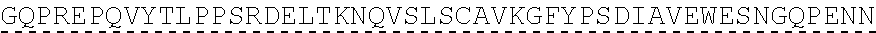

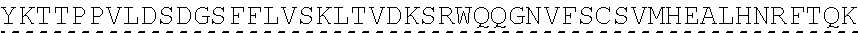

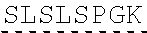

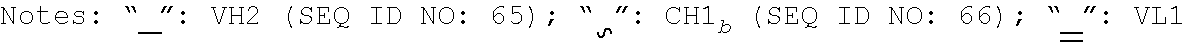

(SEQ ID NO: 84)
d) FORMAT NEW1 Chain4 (A2): anti-CD20 Light Chain
SEQ ID No.: 8
EIVLTQSPATLSLSPGERATLSCRASQSVSSYLAWYQEKPGQAPRLLIYDA
SNRATGIPARFSGSGSGTDFTLTISSLEPEDFAVYYCQQRSNWPITFGQGT

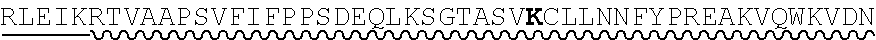

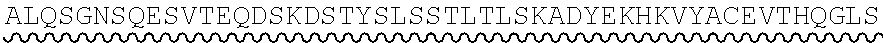

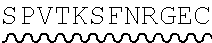

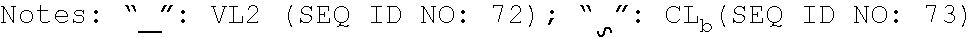

FORMAT EX2
a) FORMAT EX2 Chain2 (AH): CD20HC+CD3HC(Knob)
SEQ ID No.: 9
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

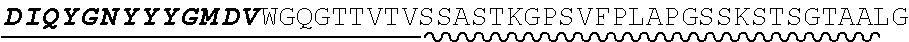

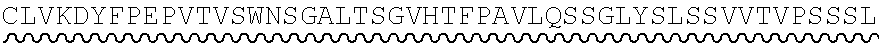

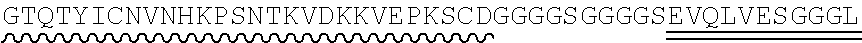

VQPGGSLRLSCAASGFTFSTYAMNWVRQAPGKGLEWVGRIRSKYNNYAT
YYADSVKGRFTISRDDSKNSLYLQMNSLRAEDTAVYYCVRHGNFGNSYV

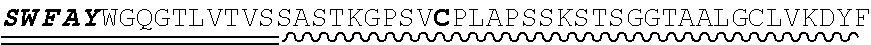

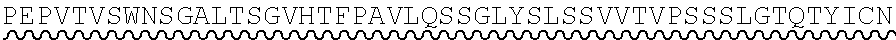

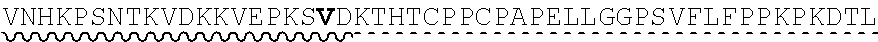

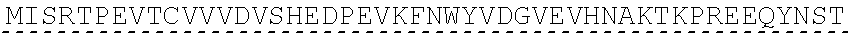

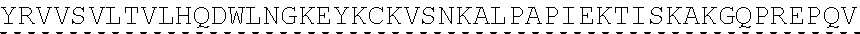

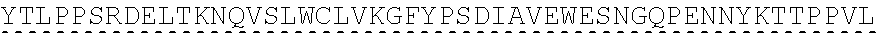

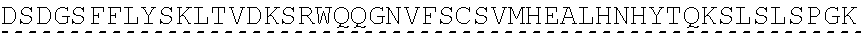

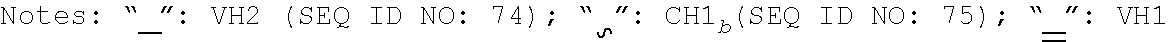

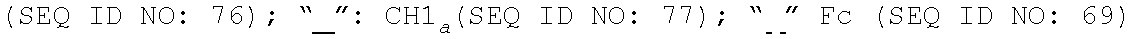

b) FORMAT EX2 Chain1 (AL): CD3 LC
SEQ ID No.: 10
QAVVTQEPSLTVSPGGTVTLTCRSSTGAVTTSNYANWVQQKPGQAPRGLI
GGTNKRAPWTPARFSGSLLGGKAALTLSGAQAEDEADYYCALWYSNLW

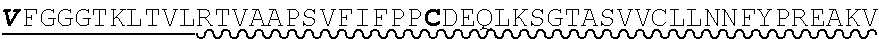

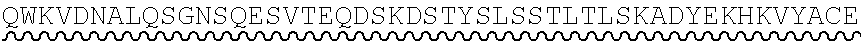

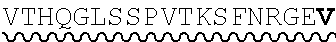

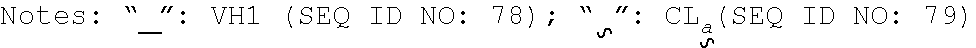

c) FORMAT EX2 Chain3 (BH): CD20 HC(Hole)
SEQ ID No.: 11
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

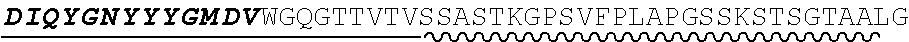

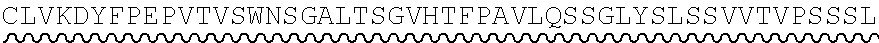

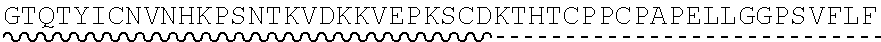

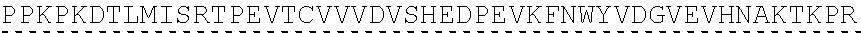

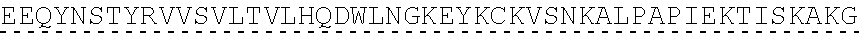

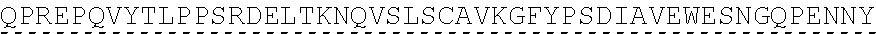

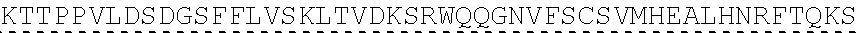

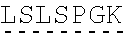

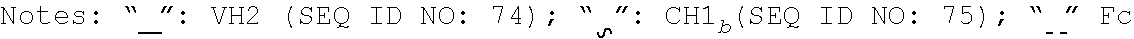

(SEQ ID NO: 84)
d) FORMAT EX2 Chain4 (BL): CD20 LC
SEQ ID No.: 12
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

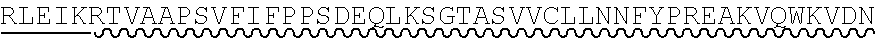

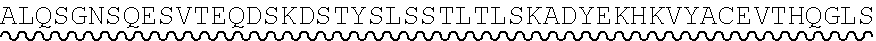

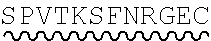

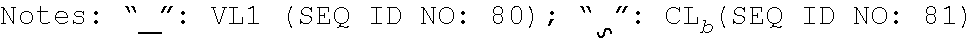

FORMAT NEW2
a) FORMAT NEW2 Chain2 (B1): anti-CD3 Heavy Chain (Knob)
SEQ ID No.: 13
EVQLVESGGGLVQPGGSLRLSCAASGFTFSTYAMNWVRQAPGKGLEWV
GRIRSKYNNYATYYADSVKGRFTISRDDSKNSLYLQMNSLRAEDTAVYY

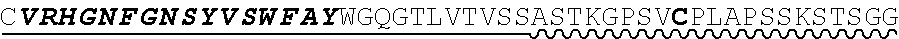

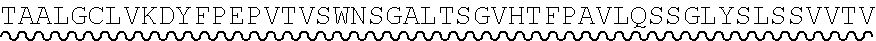

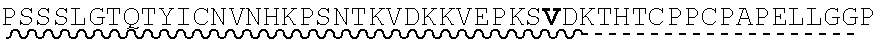

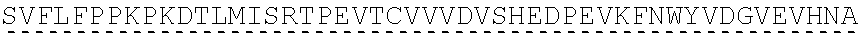

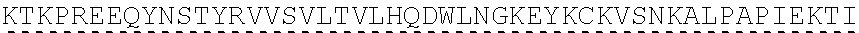

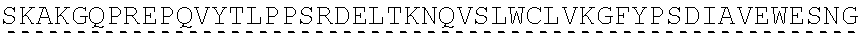

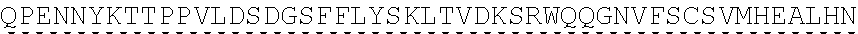

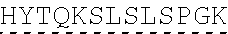

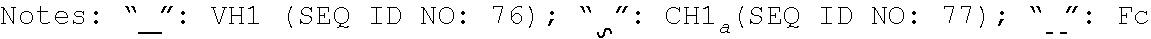

(SEQ ID NO: 69)
b) FORMAT NEW2 Chain1 (B2-A1): anti-CD20 Heavy Chain+anti-CD3 Light
Chain
SEQ ID No.: 14
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

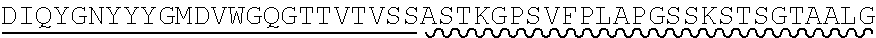

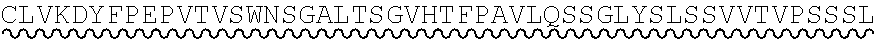

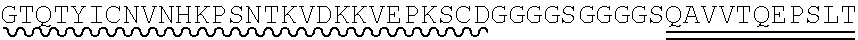

VSPGGTVTLTCRSSTGAVTTSNYANWVQQKPGQAPRGLIGGTNKRAPWT
PARESGSLLGGKAALTLSGAQAEDEADYYCALWYSNLWVFGGGTKLTVL
RTVAAPSVFIFPPCDEQLKSGTASVVCLLNNFYPREAKVQWKVDNALQS

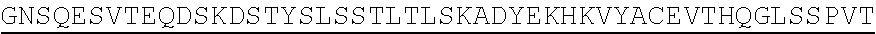

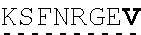

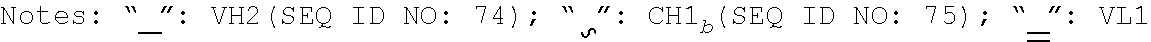

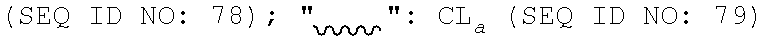

c) FORMAT NEW2 Chain3 (B2): anti-CD20 Heavy Chain (Hole)
SEQ ID No.: 15
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

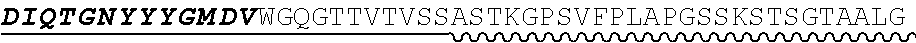

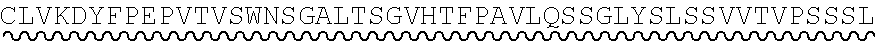

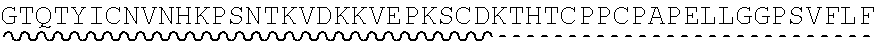

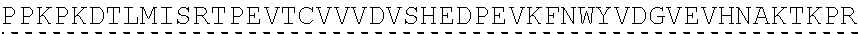

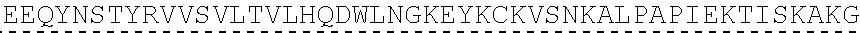

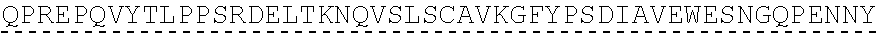

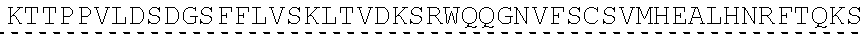

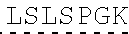

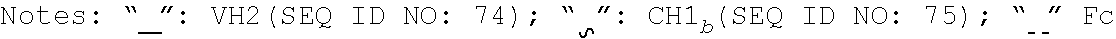

(SEQ ID NO: 84)
d) FORMAT NEW2 Chain4 (A2): anti-CD20 Light Chain
SEQ ID No.: 16
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

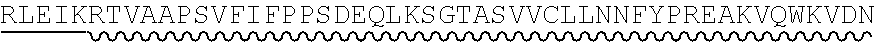

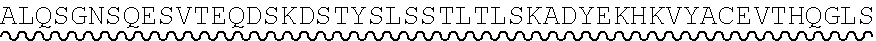

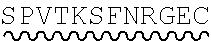

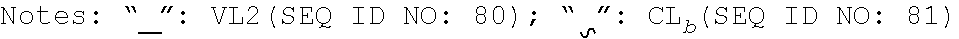

FORMAT EX3
a) FORMAT EX3 Chain2 (AH): CD20HC+CD3HC(Knob)
SEQ ID No.: 17
EVQLVESGGGLVQPGRSLRLSCAASGFTFNDYAMHWVRDAPGKGLEW
VSTISWNSGSIGYADSVKGRFTISRDNAKKSLYLQMNSLRAEDTALYYCA

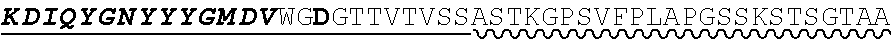

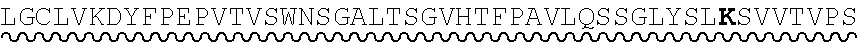

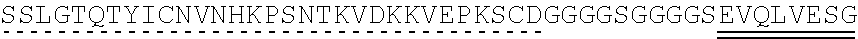

GGLVQPGGSLRLSCAASGFTFSTYAMNWVRKAPGKGLEWVGRIRSKYN
NYATYYADSVKGRFTISRDDSKNSLYLQMNSLRAEDTAVYYCVRHGNF

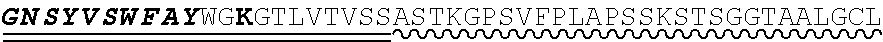

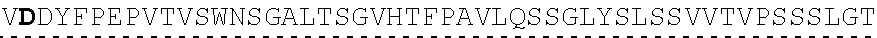

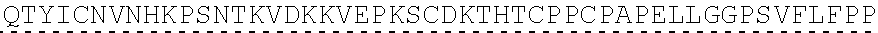

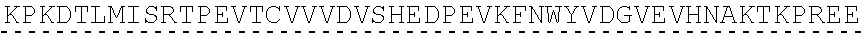

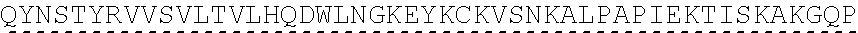

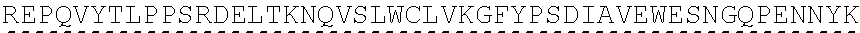

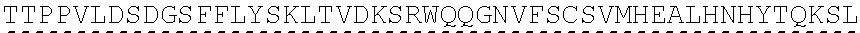

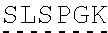

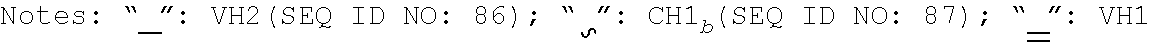

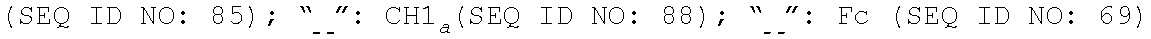

b) FORMAT EX3 Chain1 (AL): CD3LC
SEQ ID No.: 18
QAVVTQEPSLTVSPGGTVTLTCRSSTGAVTTSNYANWVQDKPGQDPRG
LIGGTNKRAPWTPARFSGSLLGGKAALTLSGAQAEDEADYYCALWYSNL

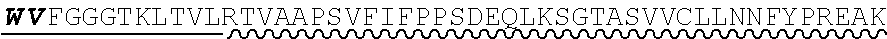

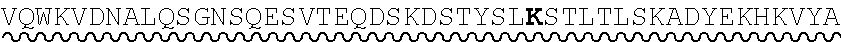

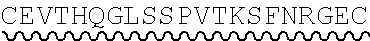

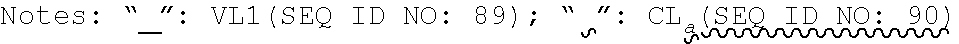

c) FORMAT EX3 Chain3 (BH): CD20HC(Hole)
SEQ ID No.: 19
EVQLVESGGGLVQPGRSLRLSCAASGFTFNDYAMHWVRDAPGKGLEW
VSTISWNSGSIGYADSVKGRFTISRDNAKKSLYLQMNSLRAEDTALYYCA

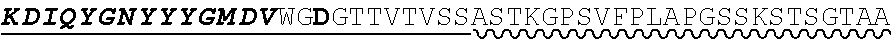

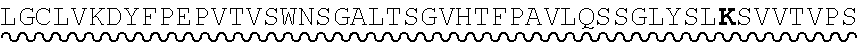

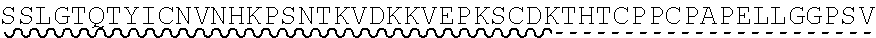

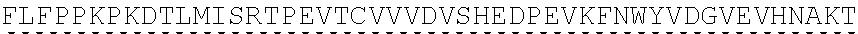

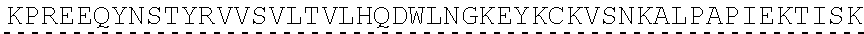

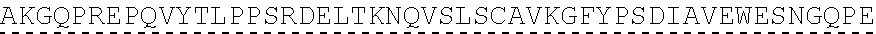

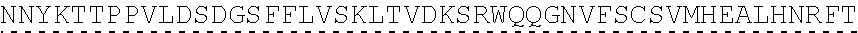

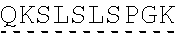

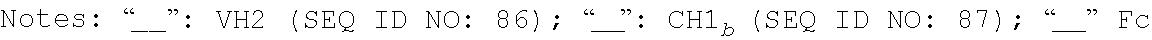

(SEQ ID NO: 84)
d) FORMAT EX3 Chain4 (BL): CD20LC
SEQ ID No.: 20
EIVLTQSPATLSLSPGERATLSCRASQSVSSYLAWYQKKPGQKPRLLIYD
ASNRATGIPARFSGSGSGTDFTLTISSLEPEDFAVYYCQQRSNWPITFGQG

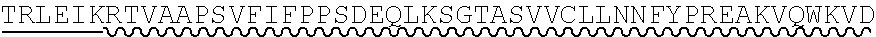

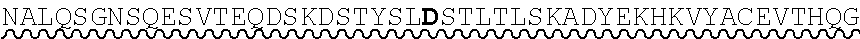

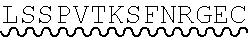

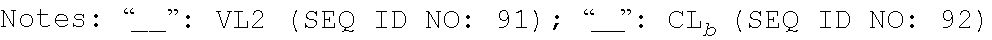

FORMAT NEW3
a) FORMAT NEW3 Chain2 (B1): anti-CD3 Heavy Chain (Knob)
SEQ ID No.: 21
EVQLVESGGGLVQPGGSLRLSCAASGFTFSTYAMNWVRKAPGKGLEW
VGRIRSKYNNYATYY ADSVKGRFTISRDDSKNSLYLQMNSLRAEDTAVY

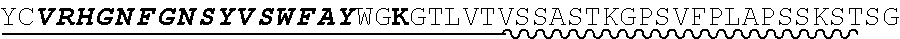

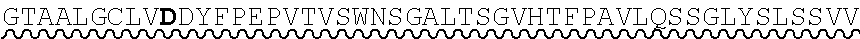

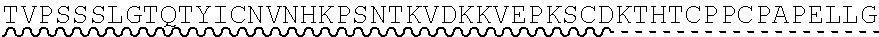

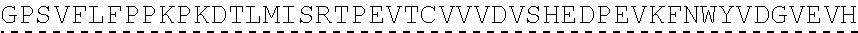

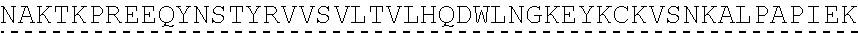

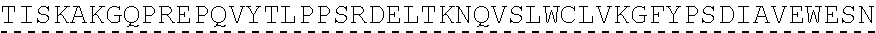

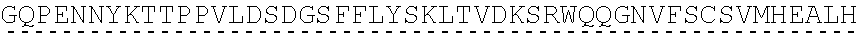

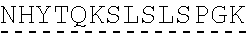

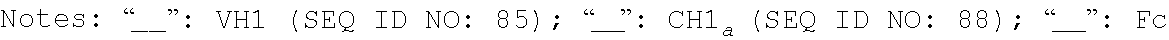

(SEQ ID NO: 69)
b) FORMAT NEW3 Chain1 (B2-A1): anti-CD20 Heavy Chain+anti-CD3 Light
Chain
SEQ ID No.: 22
EVQLVESGGGLVQPGRSLRLSCAASGFTFNDYAMHWVRDAPGKGLEW
VSTISWNSGSIGYADSVKGRFTISRDNAKKSLYLQMNSLRAEDTALYYCA

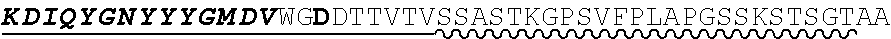

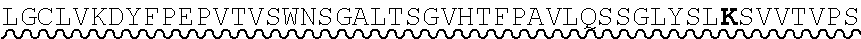

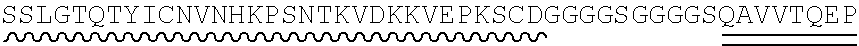

SLTVSPGGTVTLTCRSSTGAVTTSNYANWVQDKPGQDPRGLIGGTNKRA
PWTPARFSGSLLGGKAALTLSGAQAEDEADYYCALWYSNLWVFGGGTK
LTVLRTVAAPSVFIFPPSDEQLKSGTASVVCLLNNFYPREAKVQWKVDNA

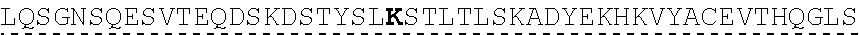

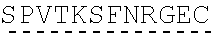

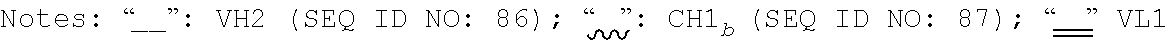

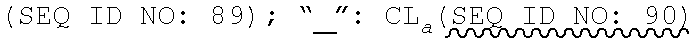

c) FORMAT NEW3 Chain3 (B2): anti-CD20 Heavy Chain (Hole)
SEQ ID No.: 23
EVQLVESGGGLVQPGRSLRLSCAASGFTFNDYAMHWVRDAPGKGLEW
VSTISWNSGSIGYADSVKGRFTISRDNAKKSLYLQMNSLRAEDTALYYCA

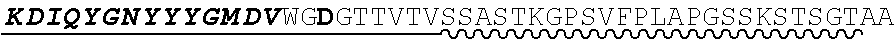

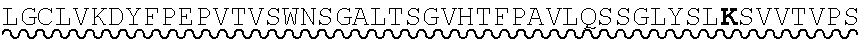

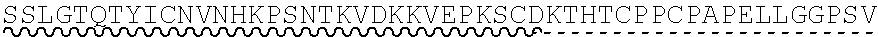

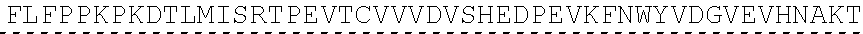

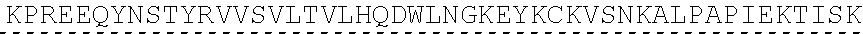

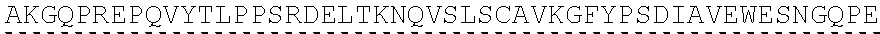

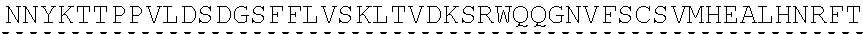

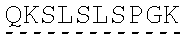

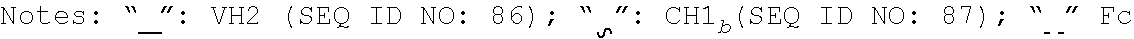

(SEQ ID NO: 84)
d) FORMAT NEW3 Chain4 (A2): anti-CD20 Light Chain
SEQ ID No.: 24
EIVLTQSPATLSLSPGERATLSCRASQSVSSYLAWYQKKPGQKPRLLIYD
ASNRATGIPARFSGSGSGTDFTLTISSLEPEDFAVYYCQQRSNWPITFGQG

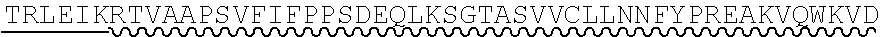

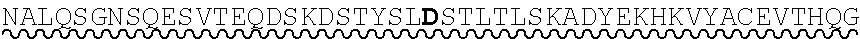

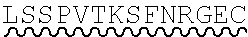

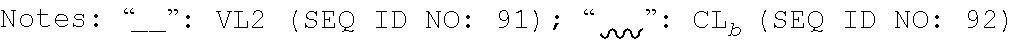

FORMAT EX4
a) FORMAT EX4 Chain2 (AH): CD20HC+CD3HC(Knob)
SEQ ID No.: 25
EVQLVESGGGLVQPGRSLRLSCAASGFTFNDYAMHWVRYAPGKGLEWV
STISWNSGSIGYADSVKGRFTISRDNAKKSLYLQMNSLRAEDTALYYCAK

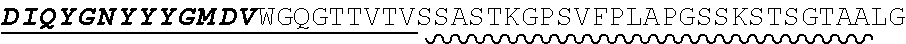

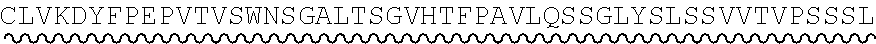

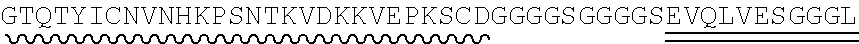

VQPGGSLRLSCAASGFTFSTYAMNWVRKAPGKGLEWVGRIRSKYNNYA
TYYADEVKGRFTISRDDSKNSLYLQMNSLRAEDTAVYYCVRHGNEGNS

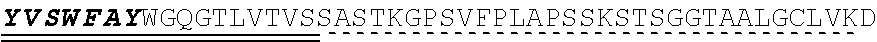

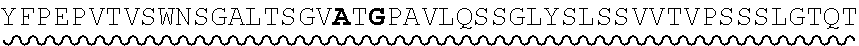

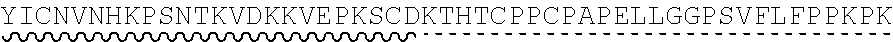

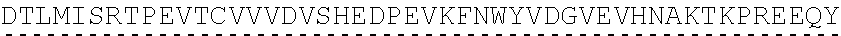

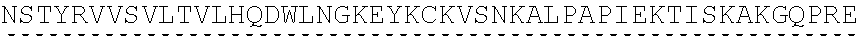

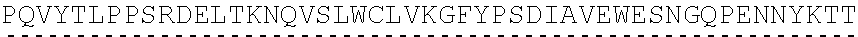

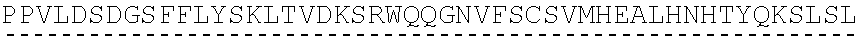

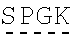

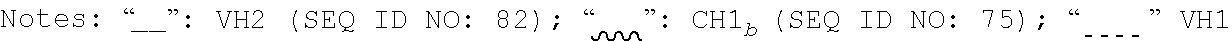

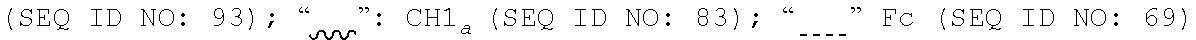

b) FORMAT EX4 Chain1 (AL): CD3 LC
SEQ ID No.: 26
RAVVTQEPSLTVSPGGTVTLTCRSSTGAVTTSNYANWVQDKPGQAPRG
LIGGTNKRAPWTPARFSGSLLGGKAALTLSGAQAEDEADYYCALWYSNL

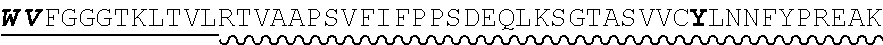

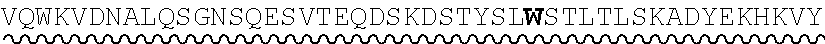

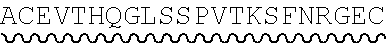

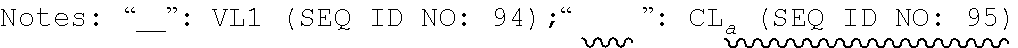

c) FORMAT EX4 Chain3 (BH): CD20HC(Hole)
SEQ ID No.: 27
EVQLVESGGGLVQPGRSLRLSCAASGFTFNDYAMHWVRYAPGKGLEWV
STISWNSGSIGYADSVKGRFTISRDNAKKSLYLQMNSLRAEDTALYYCAK

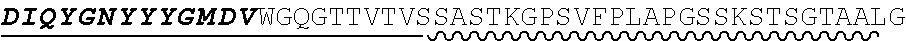

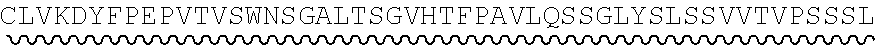

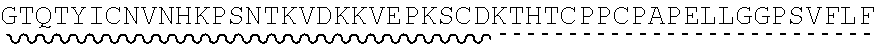

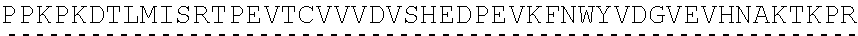

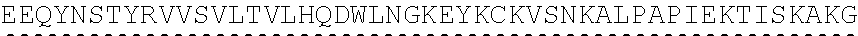

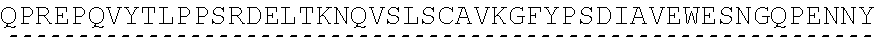

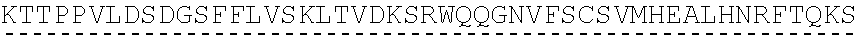

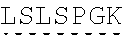

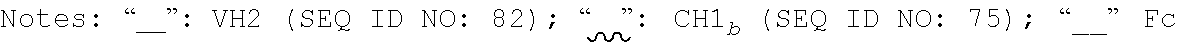

(SEQ ID NO: 84)
d) FORMAT EX4 Chain4 (BL): CD20LC
SEQ ID No.: 28
EIVLTQSPATLSLSPGERATLSCRASQSVSSYLAWYQRKPGQAPRLLIYDA
SNRATGIPARFSGSGSGTDFTLTISSLEPEDFAVYYCQQRSNWPITFGQGT

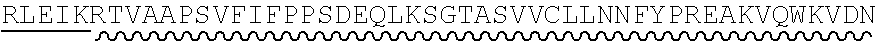

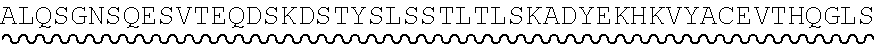

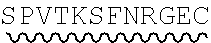

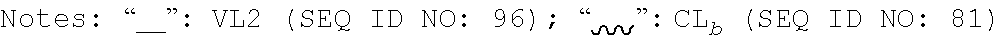

FORMAT NEW4
a) FORMAT NEW4 Chain2 (B1): anti-CD3 Heavy Chain (Knob)
SEQ ID No.: 29
EVQLVESGGGLVQPGGSLRLSCAASGFTFSTYAMNWVRKAPGKGLEW
VGRIRSKYNNYATYYADEVKGRFTISRDDSKNSLYLQMNSLRAEDTAVY

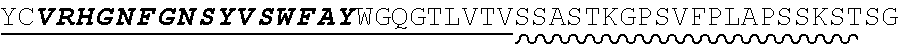

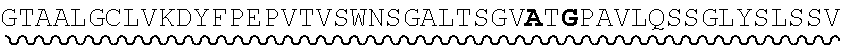

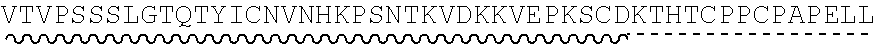

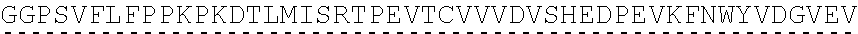

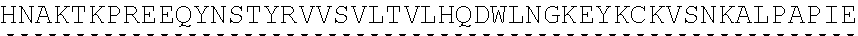

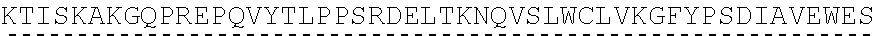

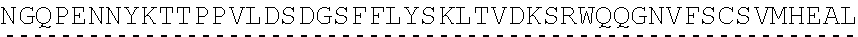

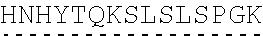

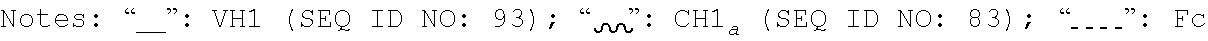

(SEQ ID NO: 69)
b) FORMAT NEW4 Chain1 (B2-A1): anti-CD20 Heavy Chain+anti-CD3
Light Chain
SEQ ID No.: 30
EVQLVESGGGLVQPGRSLRLSCAASGFTFNDYAMHWVRYAPGKGLEW
VSTISWNSGSIGYADSVKGRFTISRDNAKKSLYLQMNSLRAEDTALYYCA

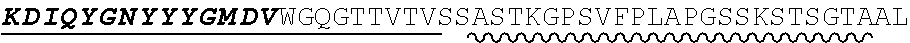

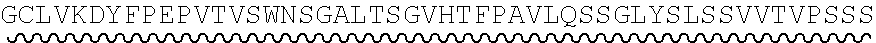

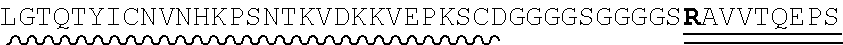

LTVSPGGTVTLTCRSSTGAVTTSNYANWVQDKPGQAPRGLIGGTNKRAP
WTPARFSGSLLGGKAALTLSGAQAEDEADYYCALWYSNLWVFGGGTKL

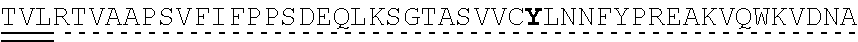

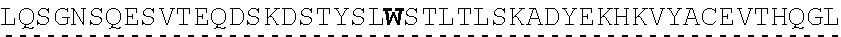

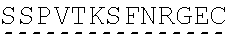

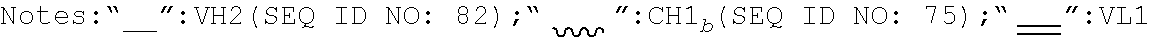

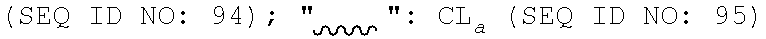

c) FORMAT NEW4 Chain3 (B2): anti-CD20 Heavy Chain (Hole)
SEQ ID No.: 31
EVQLVESGGGLVQPGRSLRLSCAASGFTFNDYAMHWVRYAPGKGLEWV
STISWNSGSIGYADSVKGRFTISRDNAKKSLYLQMNSLRAEDTALYYCAK

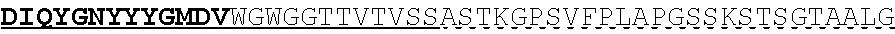

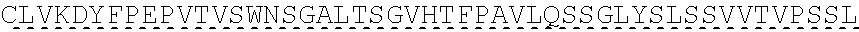

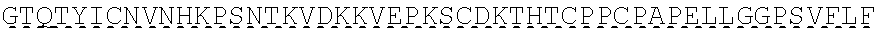

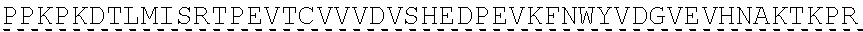

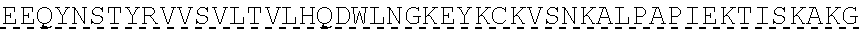

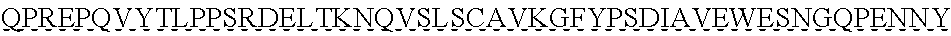

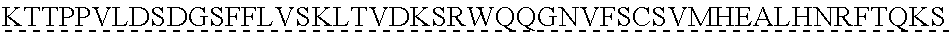

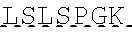

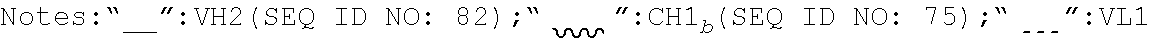

(SEQ ID NO: 84)
d) FORMAT NEW4 Chain4 (A2): anti-CD20 Light Chain
SEQ ID No.: 32
EIVLTQSPATLSLSPGERATLSCRASQSVSSYLAWYQRKPGQAPRLLIYDA
SNRATGIPARFSGSGSGTDFTLTISSLEPEDFAVYYCQQRSNWPITFGQGT

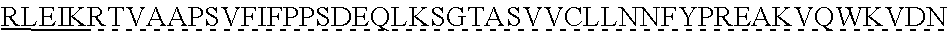

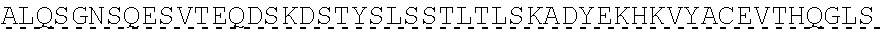

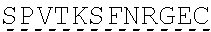

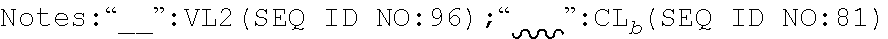

FORMAT EX5
a) FORMAT EX5 Chain2 (AH): CD20HC+CD3HC(Knob)
SEQ ID No.: 33
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

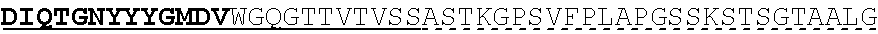

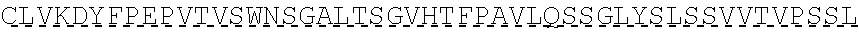

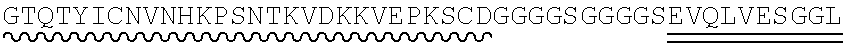

VQPGGSLRLSCAASGFTFSTYAMNWVRQAPGKGLEWVGRIRSKYNNYAT
YYADSVKGRFTISRDDSKNSLYLQMNSLRAEDTAVYYCVRHGNFGNSYV

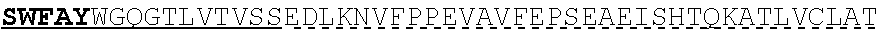

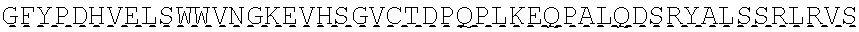

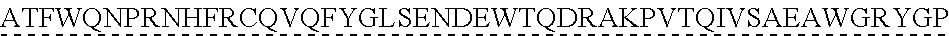

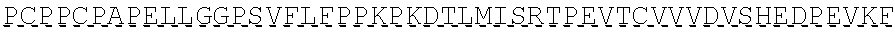

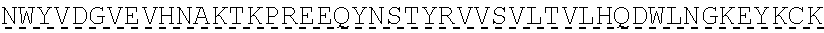

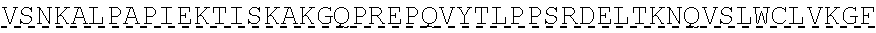

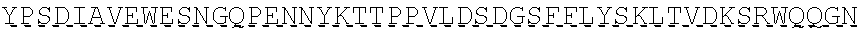

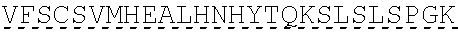

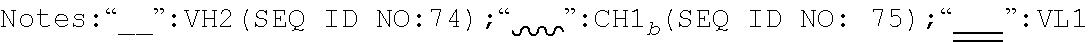

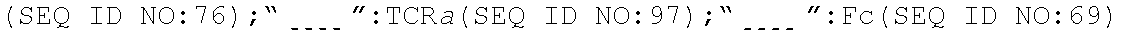

b) FORMAT EX5 Chain1 (AL): CD3LC
SEQ ID No.: 34
QAVVTQEPSLTVSPGGTVTLTCRSSTGAVTTSNYANWVQQKPGQAPRGLI
GGTNKRAPWTPARFSGSLLGGKAALTLSGAQAEDEADYYCALWYSNLW

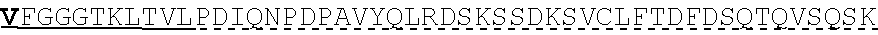

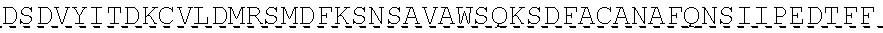

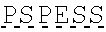

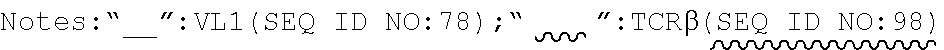

c) FORMAT EX5 Chain3 (BH): CD20HC(Hole)
SEQ ID No.: 35
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

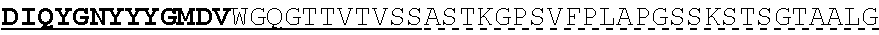

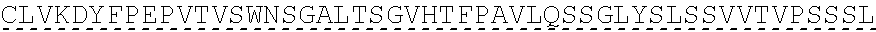

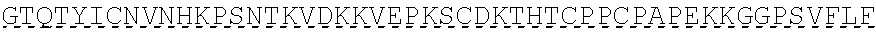

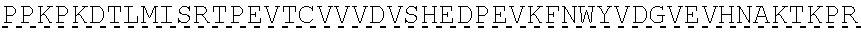

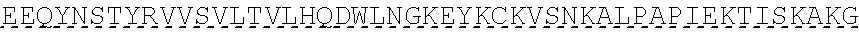

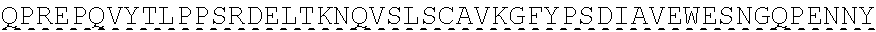

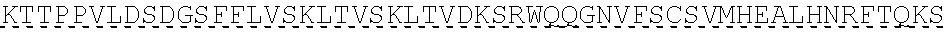

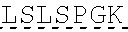

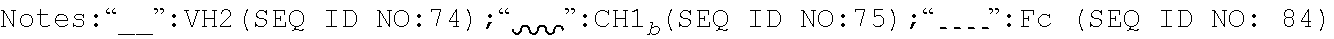

(SEQ ID NO: 84)
d) FORMAT EX5 Chain4 (BL): CD20LC
SEQ ID No.: 36
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

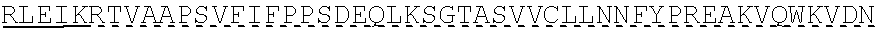

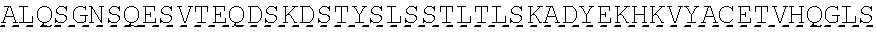

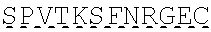

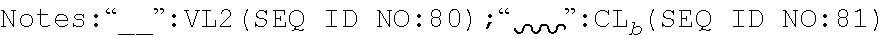

FORMAT NEW5
a) FORMAT NEW5 Chain2 (B1): anti-CD3 Heavy Chain (Knob)
SEQ ID No.: 37
EVQLVESGGGLVQPGGSLRLSCAASGFTFSTYAMNWVRQAPGKGLEWV
GRIRSKYNNYATYYADSVKGRFTISRDDSKNSLYLQMNSLRAEDTAVYY

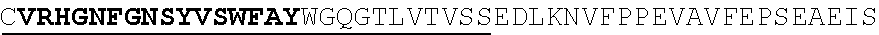

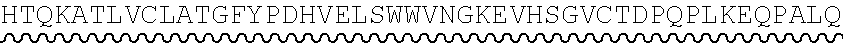

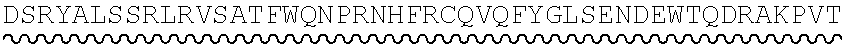

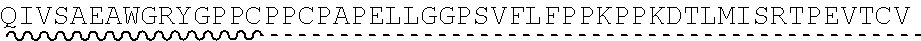

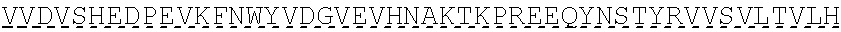

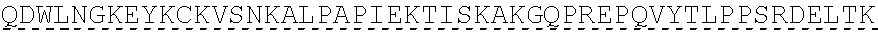

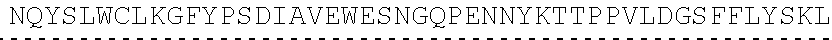

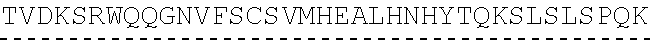

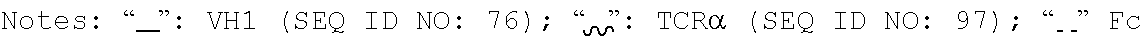

(SEQ ID NO: 69)
b) FORMAT NEW5 Chain1 (B2-A1): anti-CD20 Heavy Chain+anti-CD3
Light Chain
SEQ ID No.: 38
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

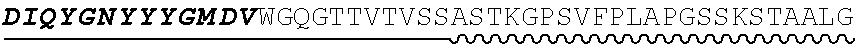

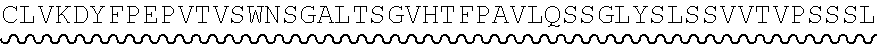

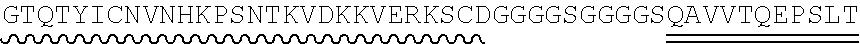

VSPGGTVTLTCRSSTGAVTTSNYANWVQQKPGQAPRGLIGGTNKRAPWT
PARFSGSLLGGKAALTLSGAQAEDEADYYCALWYSNLWVFGGGTKLTVL

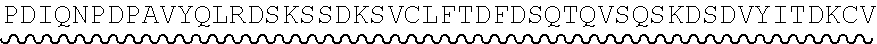

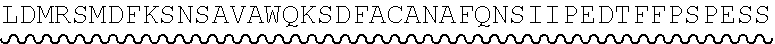

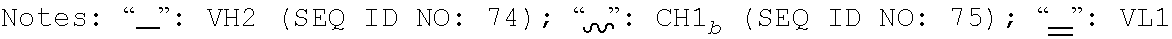

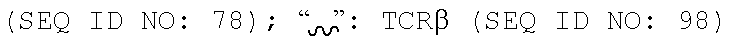

c) FORMAT NEW5 Chain3 (B2): anti-CD20 Heavy Chain (Hole)
SEQ ID No.: 39
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

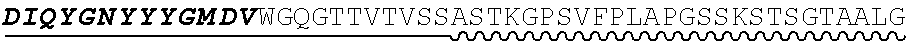

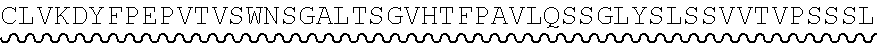

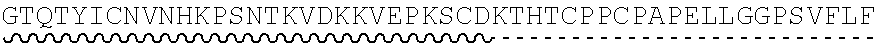

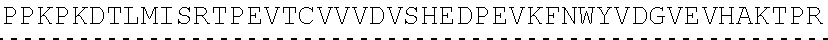

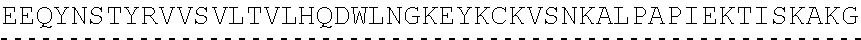

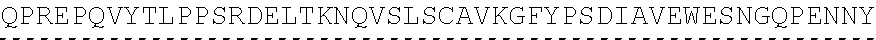

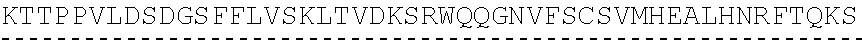

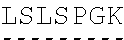

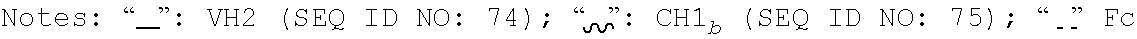

(SEQ ID NO: 84)
d) FORMAT NEW5 Chain4 (A2): anti-CD20 Light Chain
SEQ ID No.: 40
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

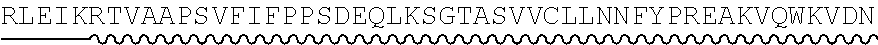

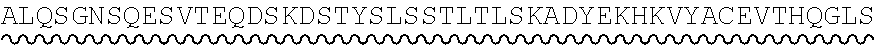

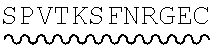

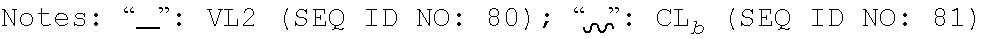

FORMAT EX6
a) FORMAT EX6 Chain2 (AH): CD20HC+CD3HC(Knob)
SEQ ID No.: 41
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

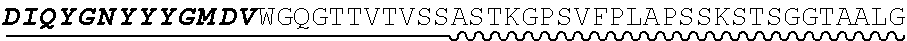

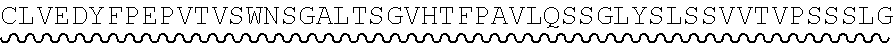

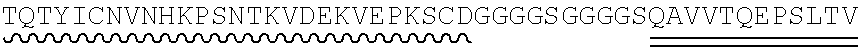

SPGGTVTLTCRSSTGAVTTSNYANWVQQKPGQAPRGLIGGTNKRAPWTP
ARFSGSLLGGKAALTLSGAQAEDEADYYCALWYSNLWVFGGGTKLTVLS

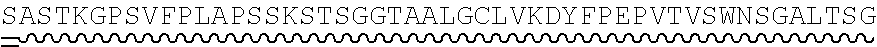

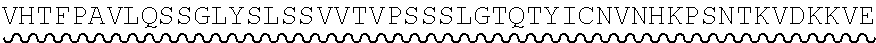

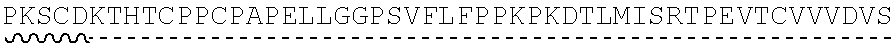

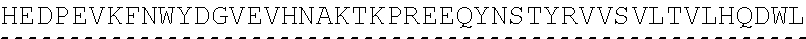

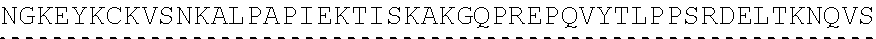

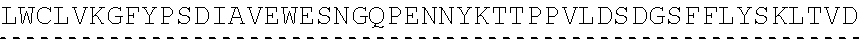

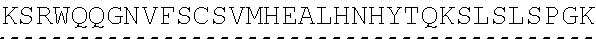

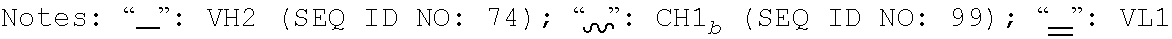

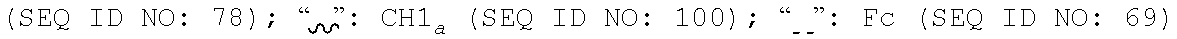

b) FORMAT EX6 Chain1 (AL): CD3LC
SEQ ID No.: 42
EVQLVESGGGLVQPGGSLRLSCAASGFTFSTYAMNWVRQAPGKGLEWV
GRIRSKYNNYATYYADSVKGRFTISRDDSKNSLYLQMNSLRAEDTAVYY

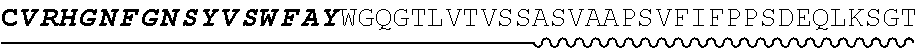

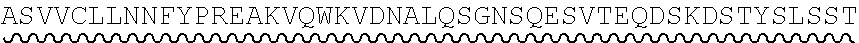

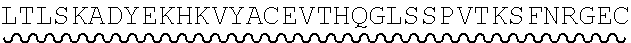

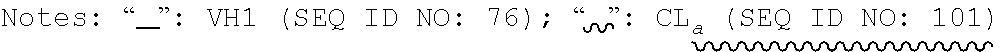

c) FORMAT EX6 Chain3 (BH): CD20HC(Hole)
SEQ ID No.: 43
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

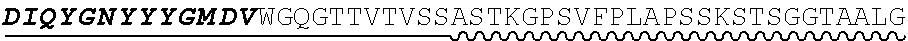

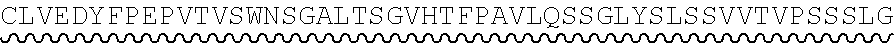

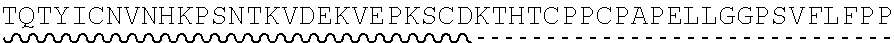

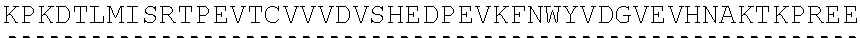

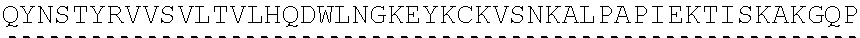

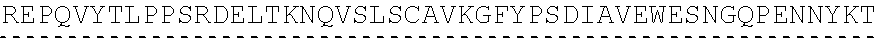

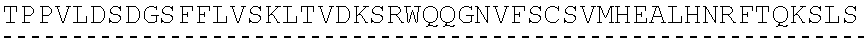

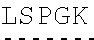

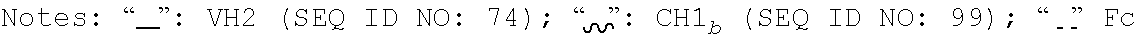

(SEQ ID NO: 84)
d) FORMAT EX6 Chain4 (BL): CD20LC
SEQ ID No.: 44
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

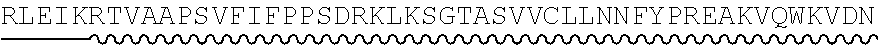

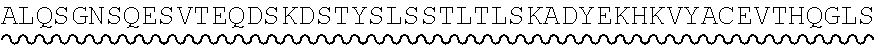

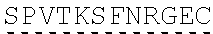

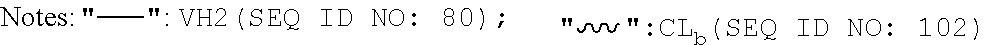

FORMAT NEW6
a) FORMAT NEW6 Chain2 (B1): anti-CD3 Heavy Chain (Knob)
SEQ ID No.: 45
QAVVTQEPSLTVSPGGTVTLTCRSSTGAVTTSNYANWVQQKPGQAPRGLI
GGTNKRAPWTPARFSGSLLGGKAALTLSGAQAEDEADYYCALWYSNLW

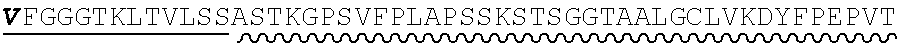

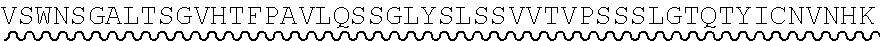

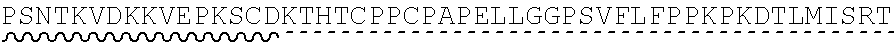

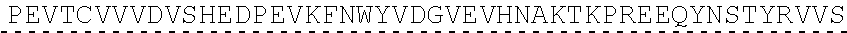

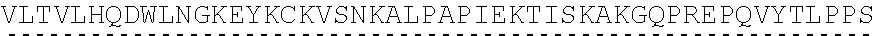

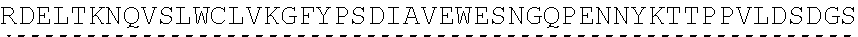

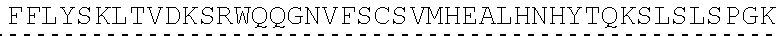

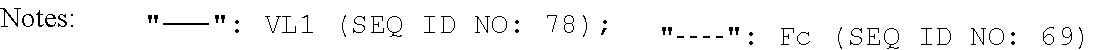

b) FORMAT NEW6 Chain1 (B2-A1): anti-CD20 Heavy Chain+anti-CD3
Light Chain
SEQ ID No.: 46
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

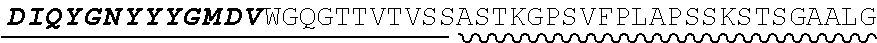

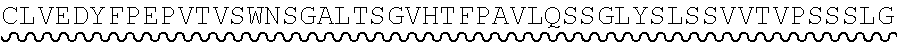

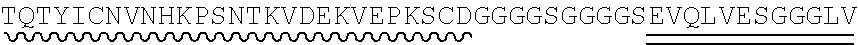

QPGGSLRLSCAASGFTFSTYAMNWVRQAPGKGLEWVGRIRSKYNNYATY
YADSVKGRFTISRDDSKNSLYLQMNSLRAEDTAVYYCVRHGNFGNSYVS

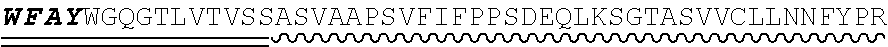

EAKVQWKVDNALQSGNSQESVTEQDSKDSTYSLSSTLTLSKADYEKHKV

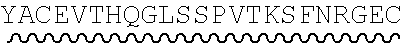

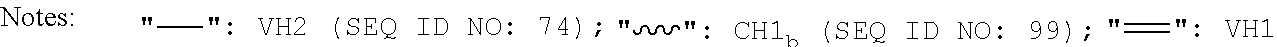

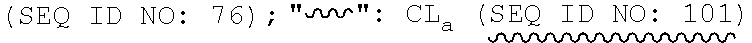

c) FORMAT NEW6 Chain3 (B2): anti-CD20 Heavy Chain (Hole)
SEQ ID No.: 47
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

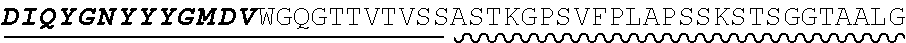

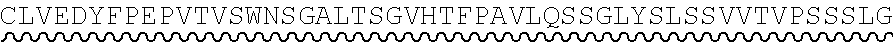

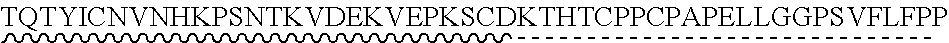

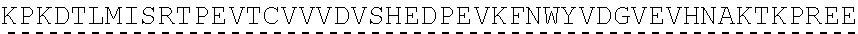

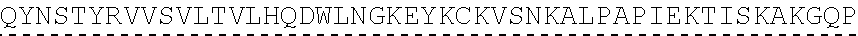

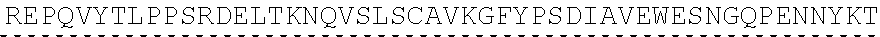

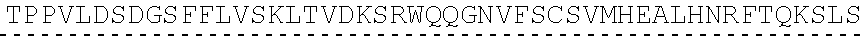

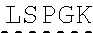

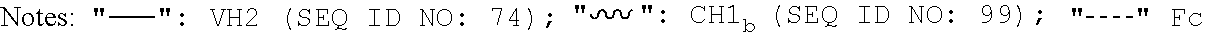

(SEQ ID NO: 84)
d) FORMAT NEW6 Chain4 (A2): anti-CD20 Light Chain
SEQ ID No.: 48
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

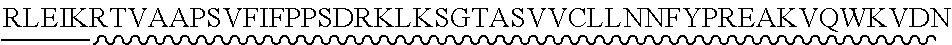

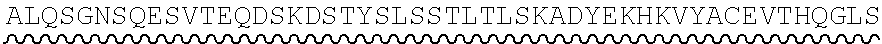

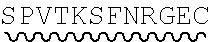

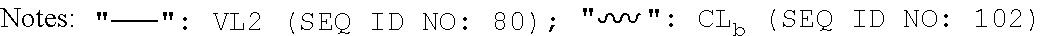

FORMAT EX7
a) FORMAT EX7 Chain2 (AH): CD20HC+CD3HC(Knob)
SEQ ID No.: 49
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

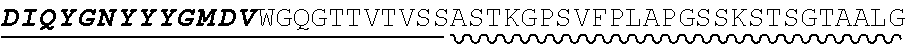

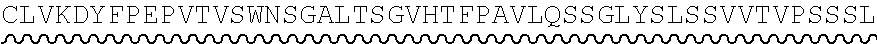

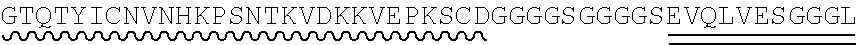

VQPGGSLRLSCAASGFTFSTYAMNWVRQAPGKGLEWVGRIRSKYNNYAT
YYADSVKGRFTISRDDSKNSLYLQMNSLRAEDTAVYYCVRHGNFGNSYV

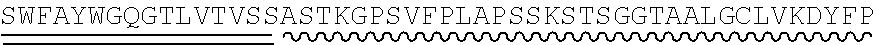

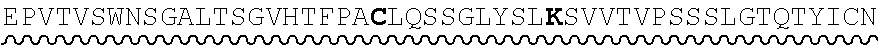

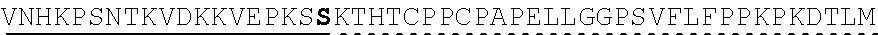

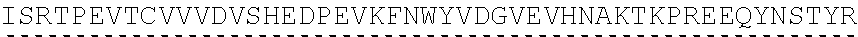

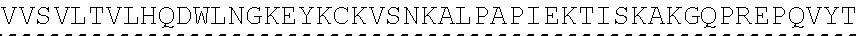

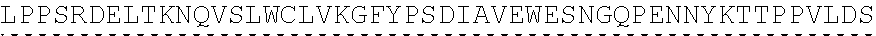

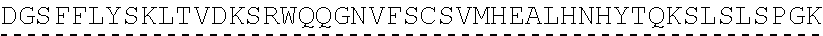

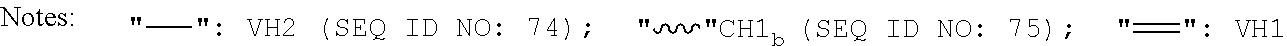

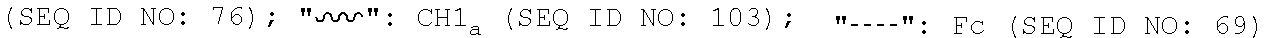

b) FORMAT EX7 Chain1 (AL): CD3LC
SEQ ID No.: 50
QAVVTQEPSLTVSPGGTVTLTCRSSTGAVTTSNYANWVQQKPGQAPRGLI
GGTNKRAPWTPARFSGSLLGGKAALTLSGAQAEDEADYYCALWYSNLW

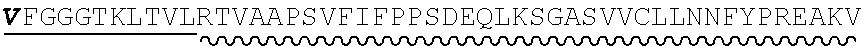

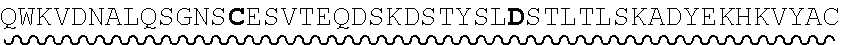

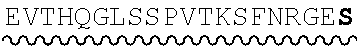

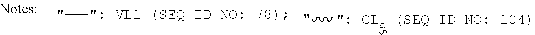

c) FORMAT EX7 Chain3 (BH): CD20 HC(Hole)
SEQ ID No.: 51
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

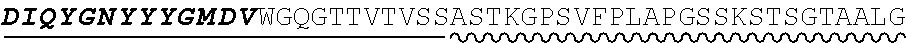

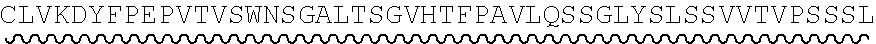

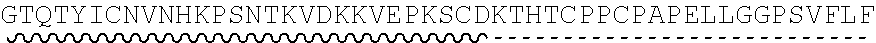

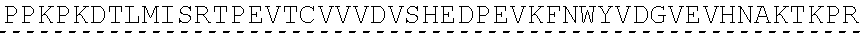

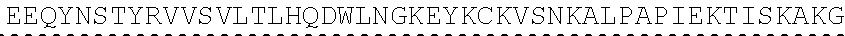

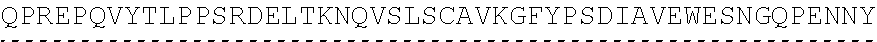

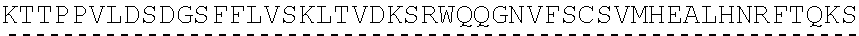

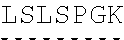

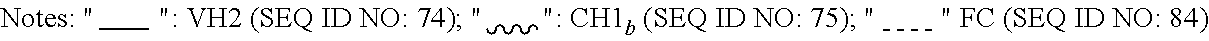

d) FORMAT EX7 Chain4 (BL): CD20 LC
SEQ ID No.: 52
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

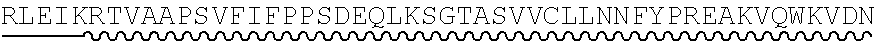

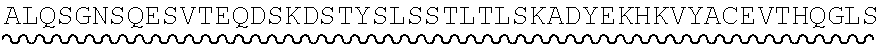

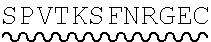

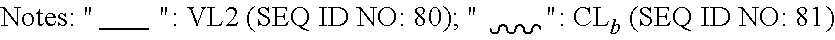

FORMAT NEW7
a) FORMAT NEW7 Chain2 (B1): anti-CD3 Heavy Chain (Knob)
SEQ ID No.: 53
EVQLVESGGGLVQPGGSLRLSCAASGFTFSTYAMNWVRQAPGKGLEWV
GRIRSKYNNYATYYADSVKGRFTISRDDSKNSLYLQMNSLRAEDTAVYY

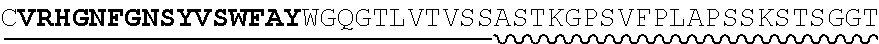

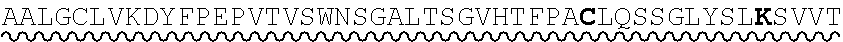

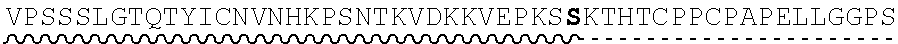

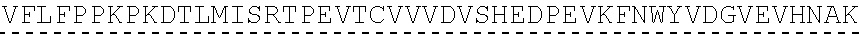

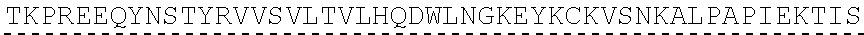

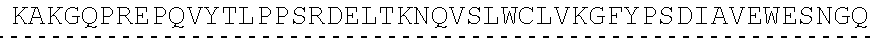

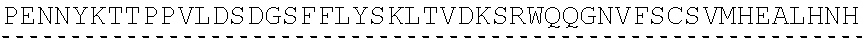

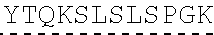

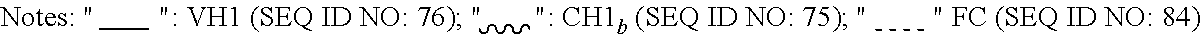

(SEQ ID NO: 69)
b) FORMAT NEW7 Chain1 (B2-A1): anti-CD20 Heavy Chain+anti-CD3
Light Chain
SEQ ID No.: 54
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

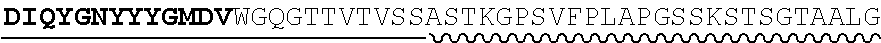

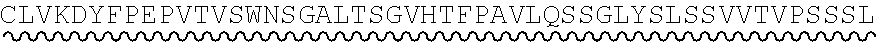

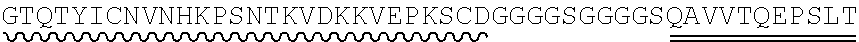

VSPGGTVTLTCRSSTGAVTTSNYANWVQQKPGQAPRGLIGGTNKRAPWT
PARESGSLLGGKAALTLSGAQAEDEADYYCALWYSNLWVFGGGTKLTVL

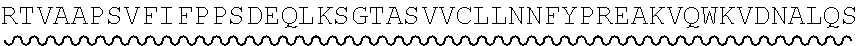

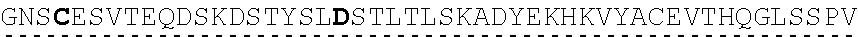

TKSENRGES

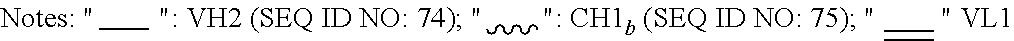

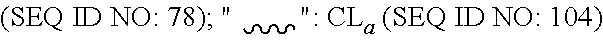

c) FORMAT NEW7 Chain3 (B2): anti-CD20 Heavy Chain (Hole)
SEQ ID No.: 55
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

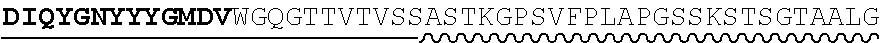

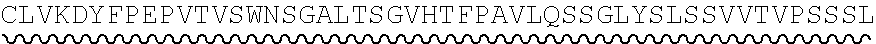

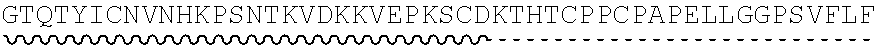

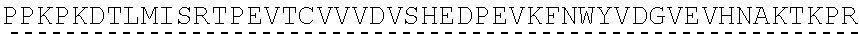

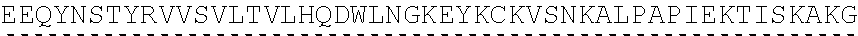

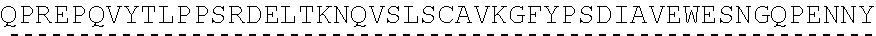

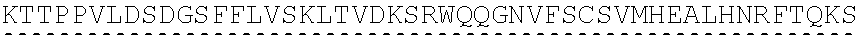

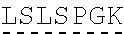

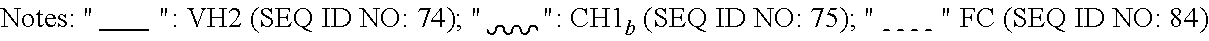

d) FORMAT NEW7 Chain4 (A2): anti-CD20 Light Chain
SEQ ID No.: 56
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

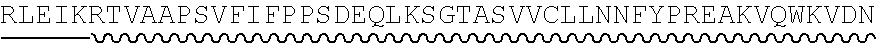

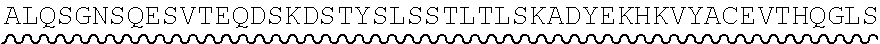

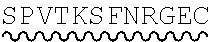

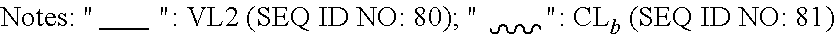

FORMAT EX8
a) FORMAT EX8 Chain2 (AH): CD20HC+CD3VHLC(Knob)
SEQ ID No.: 57
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

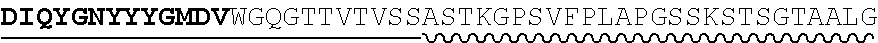

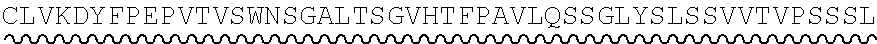

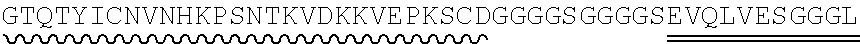

VQPGGSLRLSCAASGFTFSTYAMNWVRQAPGKGLEWVGRIRSKYNNYAT
YYADSVKGRFTISRDDSKNSLYLQMNSLRAEDTAVYYCVRHGNFGNSYV

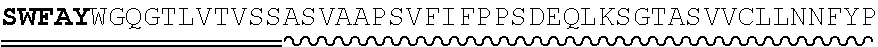

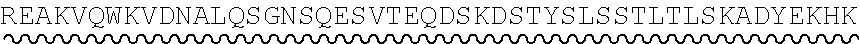

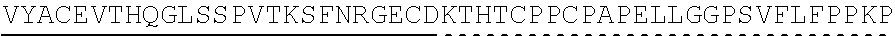

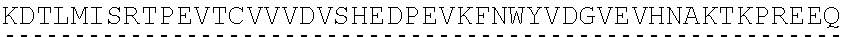

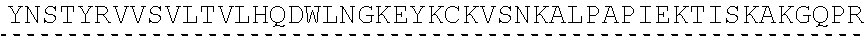

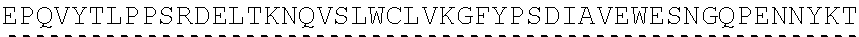

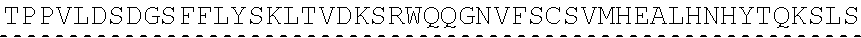

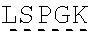

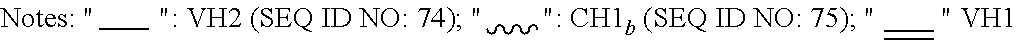

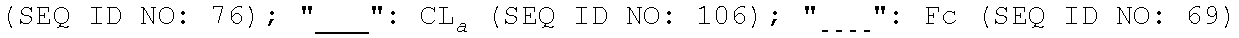

b) FORMAT EX8 Chain1 (AL): CD3LC
SEQ ID No.: 58
QAVVTQEPSLTVSPGGTVTLTCRSSTGAVTTSNYANWVQQKPGQAPRGLI
GGTNKRAPWTPARFSGSLLGGKAALTLSGAQAEDEADYYCALWYSNLW

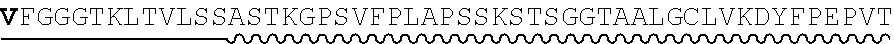

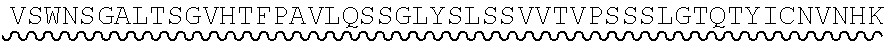

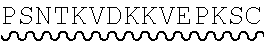

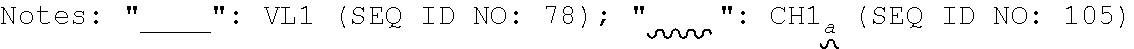

c) FORMAT EX8 Chain3 (BH): CD20HC(Hole)
SEQ ID No.: 59
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

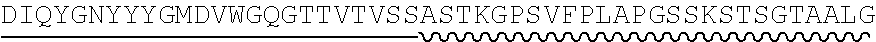

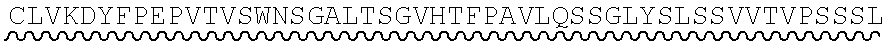

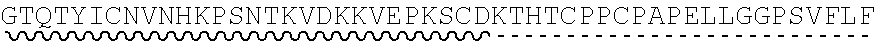

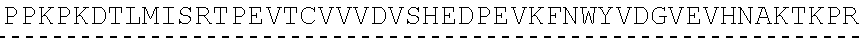

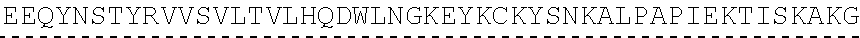

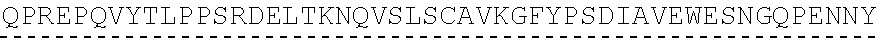

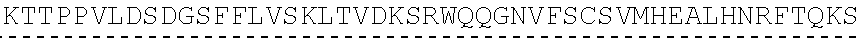

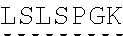

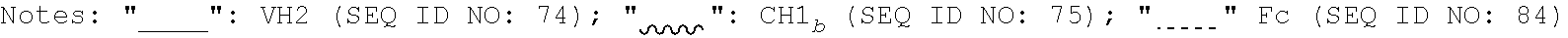

d) FORMAT EX8 Chain4 (BL): CD20LC
SEQ ID No.: 60
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITFGQGT

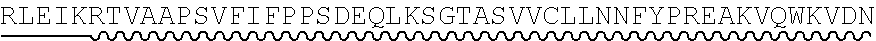

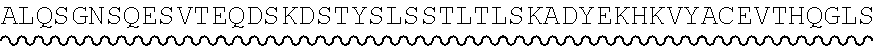

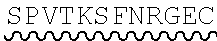

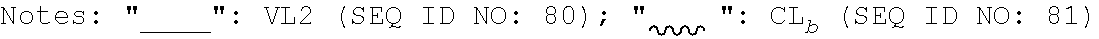

FORMAT NEW8
a) FORMAT NEW8 Chain2 (B1): anti-CD3 Heavy Chain (Knob)
SEQ ID No.: 61
EVQLVESGGGLVQPGGSLRLSCAASGFTFSTYAMNWVRQAPGKGLEWV
GRIRSKYNNYATYYADSVKGRFTISRDDSKNSLYLQMNSLRAEDTAVYY

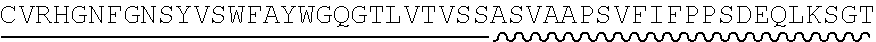

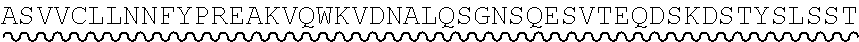

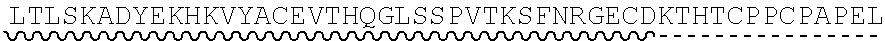

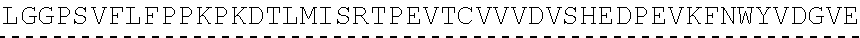

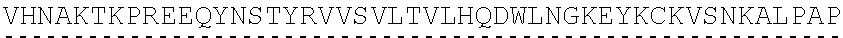

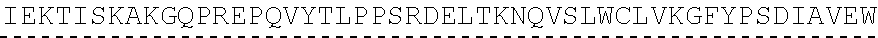

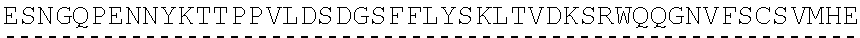

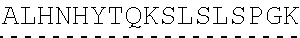

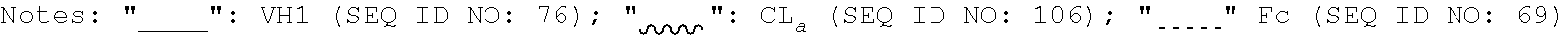

b) FORMAT NEW8 Chain1 (B2-A1): anti-CD20 Heavy Chain+anti-CD3 Light
Chain
SEQ ID No.: 62
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

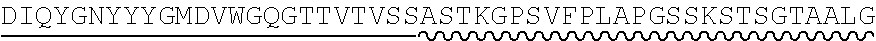

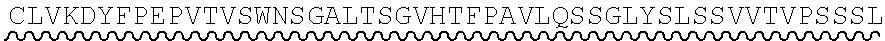

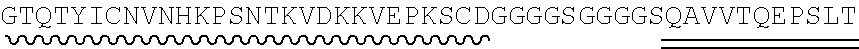

VSPGGTVTLTCRSSTGAVTTSNYANWVQQKPGQAPRGLIGGTNKRAPWT
PARFSGSLLGGKAALTLSGAQAEDEADYYCALWYSNLWVFGGGTKLTVL

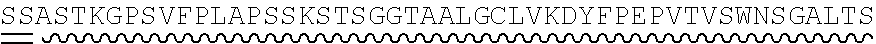

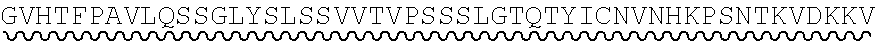

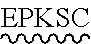

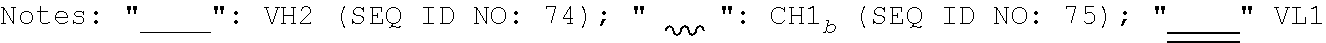

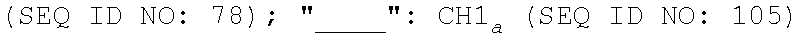

c) FORMAT NEW8 Chain3 (B2): anti-CD20 Heavy Chain (Hole)
SEQ ID No.: 63
EVQLVESGGGLVQPGRSLRLSCAASGFTFNDYAMHWVRQAPGKGLEWV
STISWNSGSIGYADSVKGRFTISRDNAKKSLYLQMNSLRAEDTALYYCAK

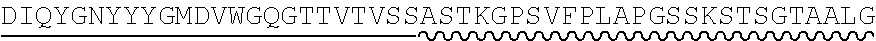

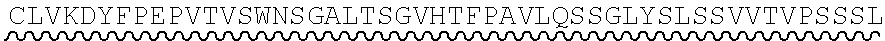

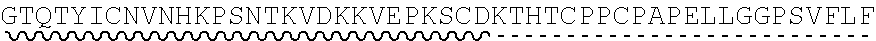

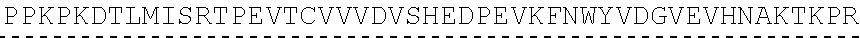

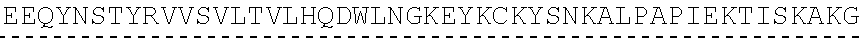

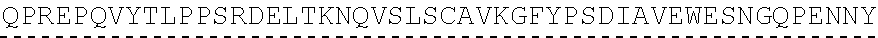

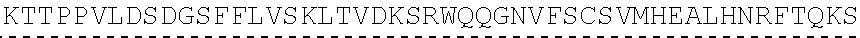

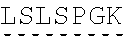

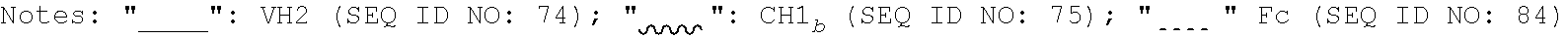

d) FORMAT NEW8 Chain4 (A2): anti-CD20 Light Chain
SEQ ID No.: 64
EIVLTQSPATLSLSPGERATLSCRASQSVSSYLAWYQQKPGQAPRLLIYDA
SNRATGIPARFSGSGSGTDFTLTISSLEPEDFAVYYCQQRSNWPITEGQGT

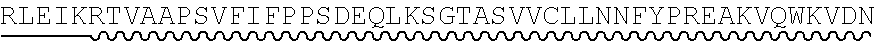

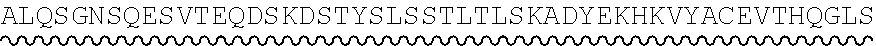

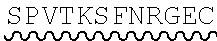

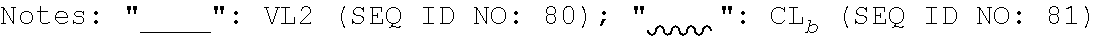

### 1.2 Gene Synthesis, Expression and Purification of the Bispecific Antibodies

Plasmids were constructed to encode each of the polypeptide chains of each of the above bispecific antibodies. The plasmids encoding for each bispecific antibody were transfected into ExpiCHO-S cells to allow expression of the polypeptide chains followed by assembly into the respective bispecific antibody. The expressed bispecific antibodies were harvested and purified with Protein-A. The specific experimental procedures are described below.

The gene sequences encoding the polypeptide chains were codon optimized using OptimumGene before proceeding to synthesis. The gene sequences were constructed in pcDNA3.4 vector, and the confirmed recombinant vector DNA was prepared for transfection by PureLink™ Hipure.

The cells were cultured in a CO₂ shaker using ExpiCHO Expression Medium, and when the ExpiCHO-S cell density reached 7×10⁶-10×10⁶ viable cells/ml and the viability rate was >95%, 25 ml of the cell suspension were transfected with ExpiFectamine™ CHO/DNA complexes containing approximately 25 ul of DNA heavy chains and light chains.

On day 8-9 after transfection, the cultures were harvested and the cell suspensions were collected, centrifuged, and further purified using chromatography. Chromatography column: 1 ml Mab Select Sure LX (GE) pre-assembled column; equilibration buffer A: 50 mM acetic acid-sodium acetate, pH 6.0; elution buffer B: 50 mM acetic acid-sodium acetate, pH 3.6; neutralization buffer C: 1M Tris-HCl, pH 8.5; flow rate: 1 ml/min; gradient: 0-100% B linear gradient elution. After separation, the eluate was collected at a volume of 1 ml/tube, and 0.1 ml of neutralization buffer C was added to each 1 ml fraction, pH about 6-7.

### 1.3 the Expression Results of Each Bispecific Antibody

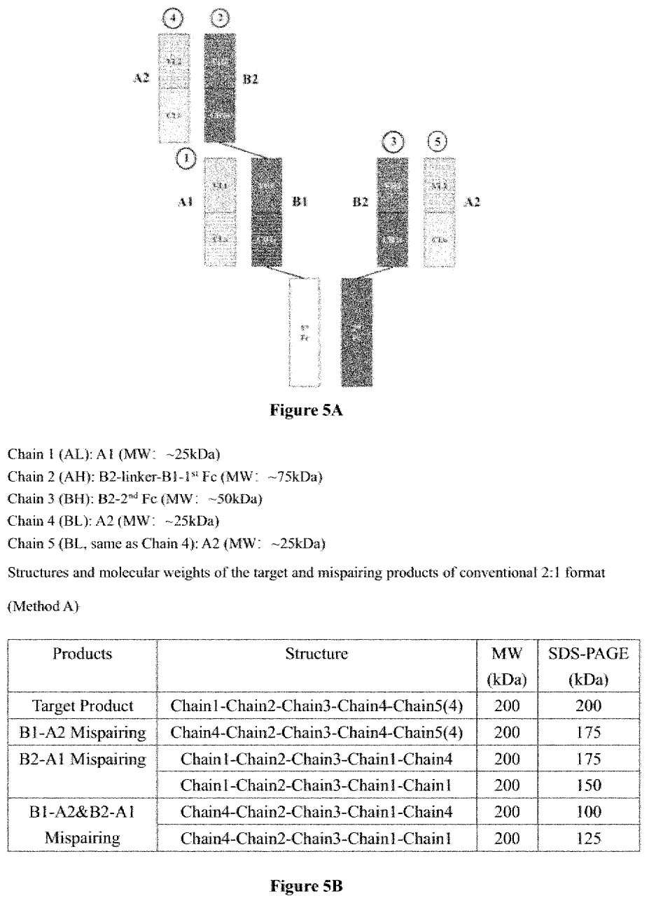

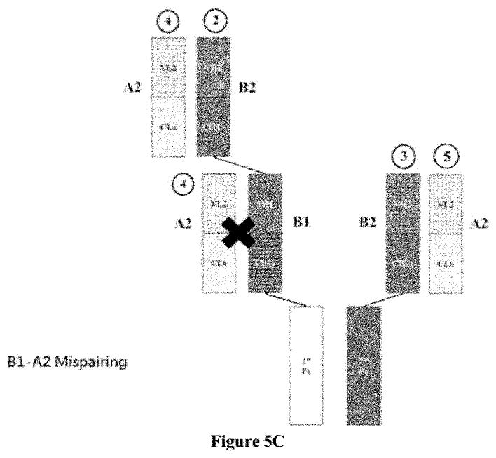

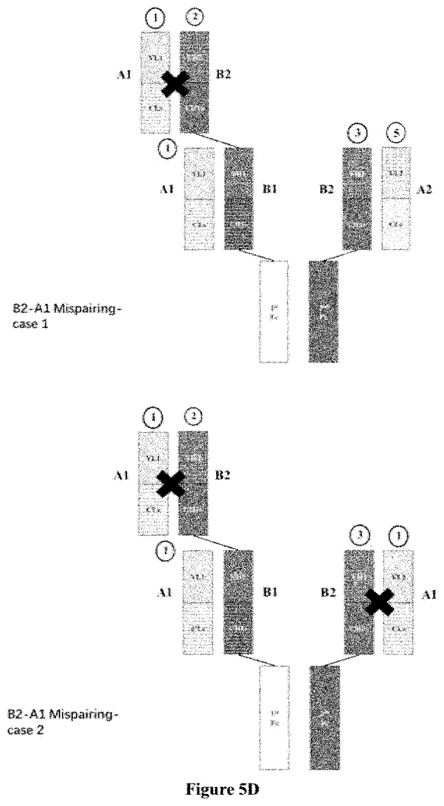

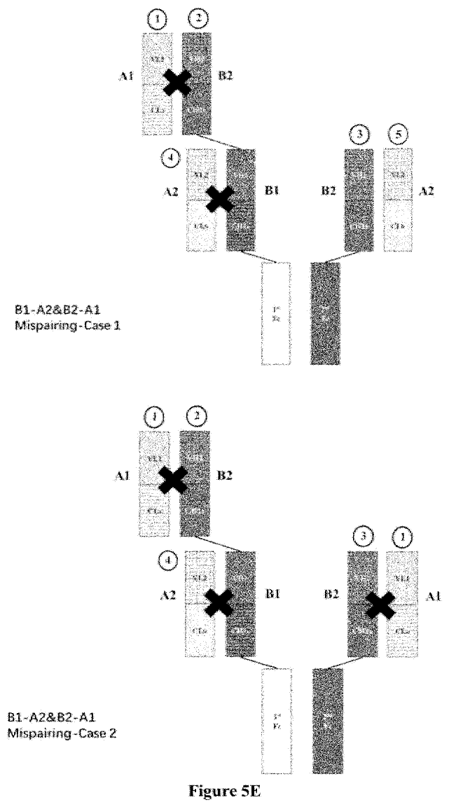

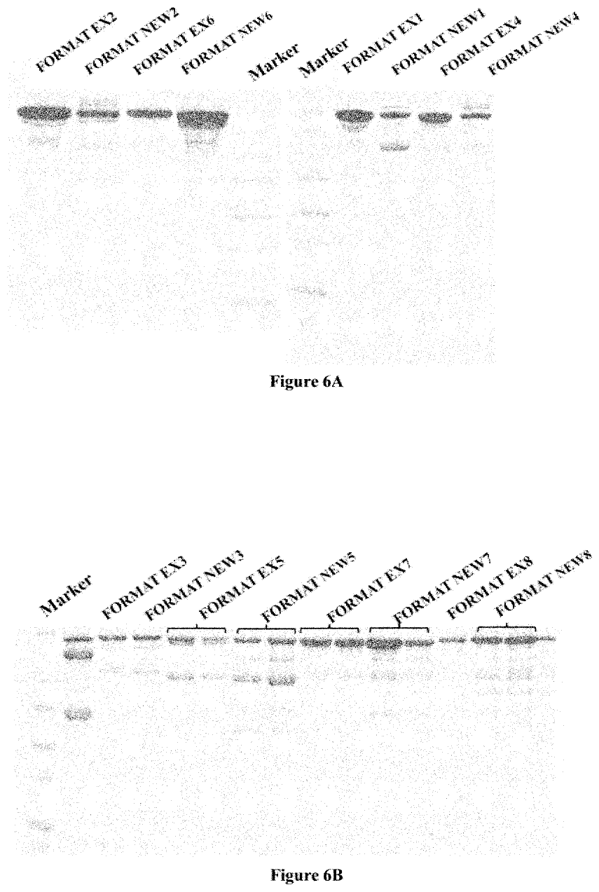

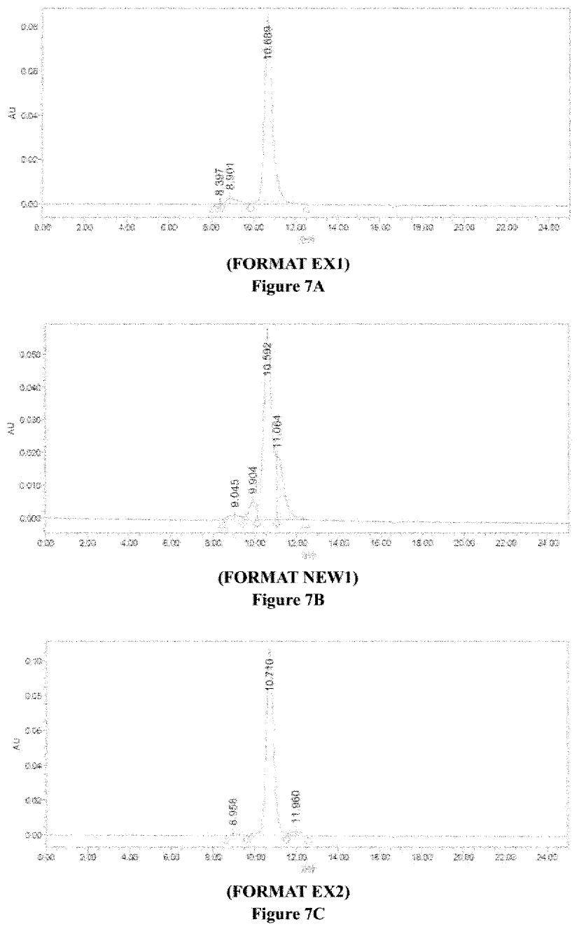

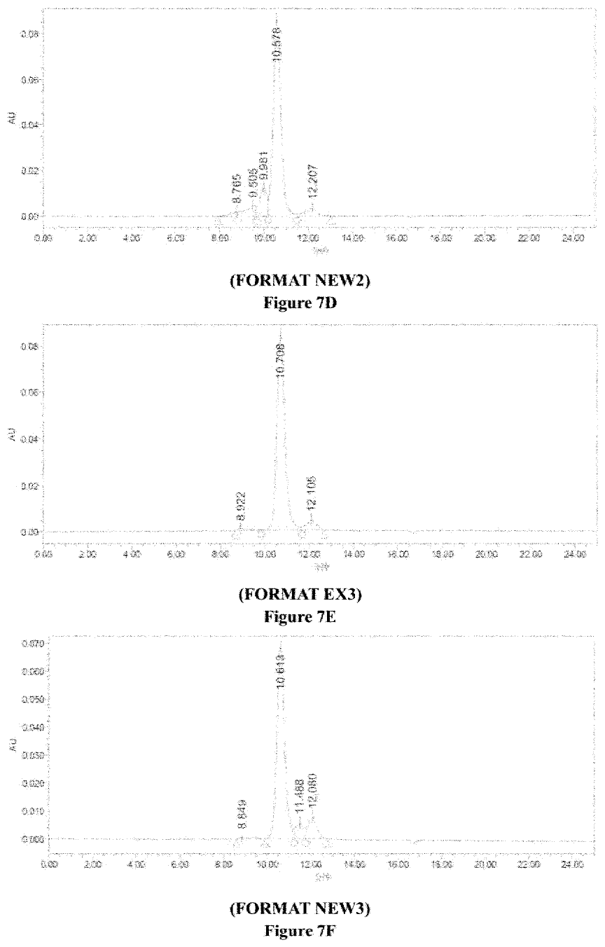

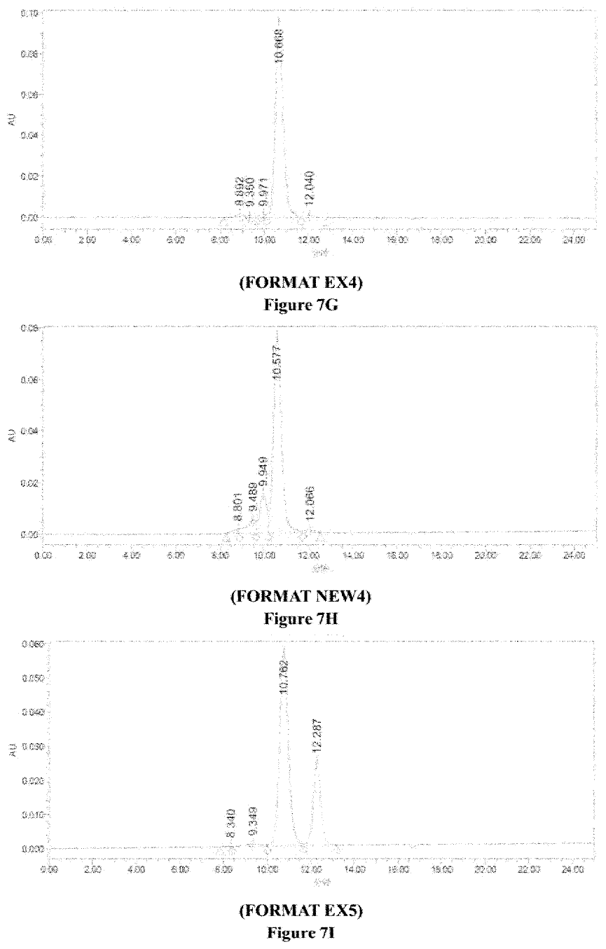

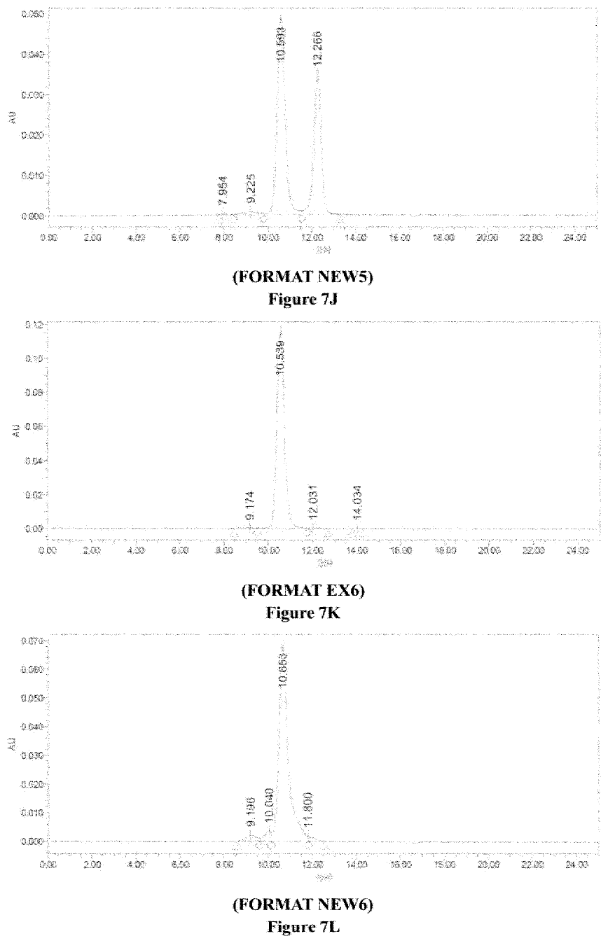

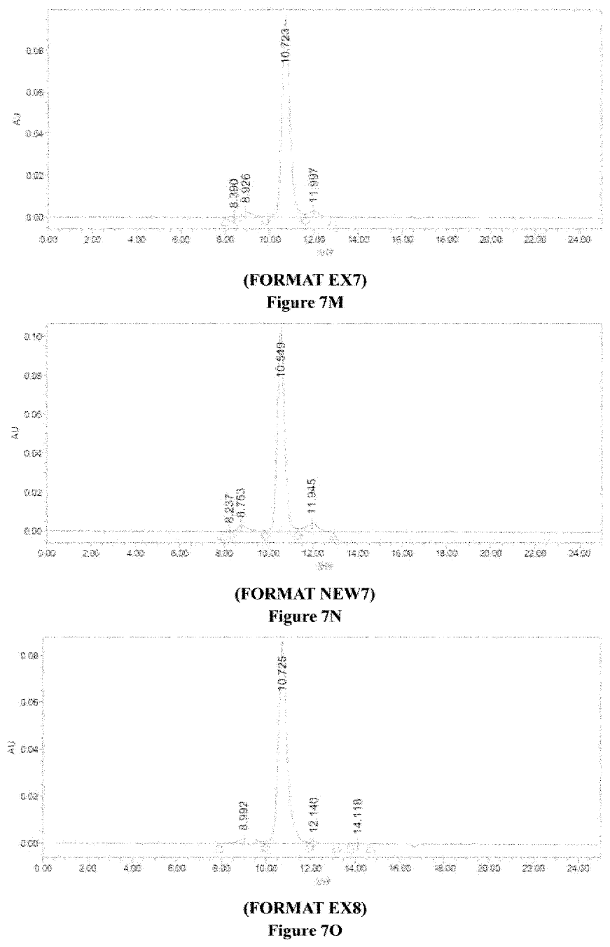

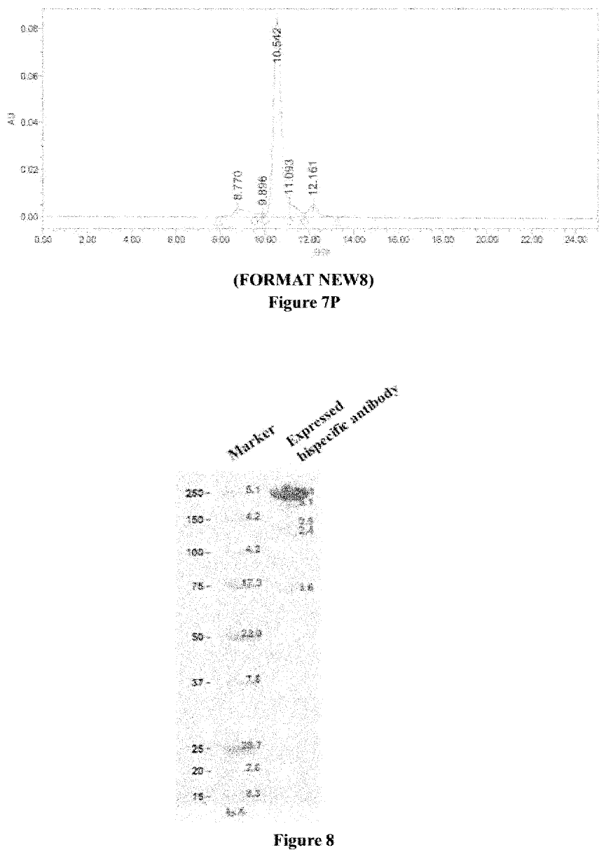

The purified antibodies were analyzed and identified by SEC-HPLC (see FIGS. 7A to 7P) and SDS-PAGE (see FIGS. 6A and 6B), respectively.

1). SDS-PAGE: The sample was mixed with 4× sample buffer (15 μl±5 μl) in an Eppendorf tube and heated at 100° C. for 5-10 min, centrifuged, with the supernatant collected. Then, 5 μg of the treated sample was taken with a pipette and carefully added to each well of the gel sequentially, and marker was added to one of the wells. The electrode chamber was placed on the electrophoresis tank, with the power on, the positive and negative poles aligned, and the voltage set to 150 V. After running for 5 min, the voltage was adjusted to 200 V. Electrophoresis was run again for about 35 min and then turn off the power to stop. A Bio-rad gel imager was used to scan and take pictures of the non-stained SDS-PAGE gels, and the pictures were analyzed using Image Lab5.2.1 to calculate protein purity.

2). Capillary electrophoresis sodium dodecyl sulfate CE-SDS: The sample was diluted to 2.5 mg/ml with ultrapure water and added to 55 μL Sample Buffer, followed by 2 μL 10 kDa IS and 5 μL 250 mM IAM, and mixed well. The samples were next heated in a metal bath at 70±2° C. for 10±2 min, then cooled to room temperature in a water bath and transferred to the accompanying sample tube, labeled and ready for sampling.

3). Size Exclusion-High Performance Liquid Chromatography (SEC-HPLC):

The samples were centrifuged at 15° C. with 10000 rpm for 5 min. The supernatant was taken and analyzed. The relevant SEC chromatographic parameters are listed below:
Mobile phase A: 200 mM PBS pH 6.9;
Mobile phase B: ultrapure water;
Flow rate: 0.7 ml/min;
Wavelength: 280 nm;
Column temperature: 25° C.;
Sample analysis time: 25 min;
Injection size: 20 μg.

### 1.4 Analysis Results

### Conventional 2:1 Format (FORMAT EX)

According to the SEC-HPLC results (FIGS. 7A, 7C, 7E, 7G, 7I, 7K, 7M and 7O), all of the samples appeared to show a clear single 200 kDa size major peak except for the FORMAT EX5 construct (FIG. 7I), suggesting that almost all samples have high purity. However, this result was inconsistent with the SDS-PAGE results (see, FIGS. 6A and 6B), which showed multiple protein bands in the sample, indicating that the samples contained low molecular impurities and therefore still need purification.

The reason why the SEC-HPLC results were inconsistent with the SDS-PAGE results is that, most of the A1-B2 and A2-B1 mispairing products in the samples of the FORMAT EX antibodies (FORMAT EX1 to FORMAT EX8) have molecular weights around 200 kDa, which is identical or very close to that of the intended product, and therefore the SEC-HPLC method cannot identify these mispaired products present in the samples. In contrast, such mispaired products can be revealed by the SDS-PAGE method, because the A1-B2 and A2-B1 mispairing products in FIG. 5B were not covalently bound by disulfide bonds, and would undergo dissociation during the SDS-PAGE process. In the present study, the SDS-PAGE results showed that the presence of 175 kDa or even 150 kDa products near the 200 kDa band could be seen for FORMAT EX1, FORMAT EX2, FORMAT EX3, FORMAT EX4, FORMAT EX5, FORMAT EX7 and FORMAT EX8, but not for FORMAT EX6. These mispairing products were not seen in the SEC-HPLC results, and can be extremely difficult to remove by conventional purification process, due to the similarity in physiochemical properties to the corresponding cognate paired target product.

### Novel 2:1 Format (FORMAT NEW)

As shown on SDS-PAGE results (see, FIGS. 6A and 6B), the impurities exhibiting a long distance from the target bands were dominated by 150 kDa on SDS-PAGE. These results are more consistent with the SEC-HPLC results (see, FIGS. 7B, 7D, 7F, 7H, 7J, 7L, 7N and 7P), i.e., the main peak of 200 KDa on the SEC-HPLC profile is a single target product, while the A1-B2 as well as A2-B1 mispairing products (see FIG. 4B) are easier to separate by SEC-HPLC due to their large difference in molecular weight with the target product, and the final products are also more convenient to purify due to the significant difference in physiochemical properties.

### Example 2

The anti-CD20×CD3 bispecific molecule FORMAT New7 was constructed and purified using conventional methods, demonstrating the convenience of the present invention for purification.

### 2.1 Antibody Construction:

Chain 1 (AL): B2-linker-A1 (MW: about 50 kDa)
Chain 2 (AH): B1-1st Fc (MW: about 50 kDa)
Chain 3 (BH): B2-2nd Fc (MW: about 50 kDa)
Chain 4 (BL): A2 (MW: about 25 kDa)

The following mutations were introduced into CH1 of B1: V173C, S183K, C220S.

The following mutations were introduced into CL of A1: Q160C, S176D, C214S.

### 2.2 The Expression of the Anti-CD20×CD3 FORMAT New7 Bispecific Antibody:

Anti-CD20×CD3 FORMAT New7 is a bispecific antibody, consisting of 2 pairs of different light and heavy chains. Two stable transfer vectors were used to co-transfect host cells with CHO-K1 cells. After transfection, cells were plated at a density of 2×10e4 cells/well, and cultured in the presence of selection pressure in a CO₂ incubator. After about 3 weeks of culture, supernatants were taken from the cell culture to test for binding to CD20 and CD3, respectively. The supernatants showing high binding activity to CD20 and CD3 were identified, and the corresponding cell clones were expanded into 24-well cell culture plates. After 4 to 6 days, supernatants were tested for binding activity to CD20 and CD3. Cell clones with higher binding activity to CD20 and CD3 were expanded, and finally the stable-transfected cell lines were selected and expanded. Stable cell lines were passaged and cultured in shake flasks for 3 days, tested for binding activity to CD20 and CD3, and Fed-batch evaluation and cryopreservation were performed based on cell growth and expression. The expression and quality of the Feb-batch samples were tested (SEC, non-reduced CE-SDS, CEX, SDS-PAGE) to evaluate the growth, metabolism, productivity and product quality of the stable cell pool, and the stable cell pool was finally selected for subsequent monoclonal cell line screening.

The cell line mentioned above was plated into 96-well plates by single cell sorter with 1 cell per well, cultured in CO₂ incubator and photographed by sweeping the plates with Solentim Cell Metric. Monoclonal cells were selected for Fed-batch evaluation and cell cryopreservation. The expression and quality analysis (SEC, nrCE-SDS, iCIEF, N-Glycan, molecular weight) were performed to evaluate the growth, metabolism, productivity and product quality of the stable cell pool, and 6 monoclonal cell lines were selected for RCB banking.

The selected monoclonal cell lines were revived, amplified, inoculated and cultured in shake flasks for 14 days. The supernatants were harvested for purification and analysis.

### 2.2.1 Analysis Methods:

1). SDS-PAGE: The supernatant sample was mixed with 4× sample buffer (15 μl+5 μl) in an Eppendorf tube and heated at 100° C. for 5-10 min, centrifuged, with the supernatant collected. Then, 5 g of the treated sample was taken with a pipette and carefully added to each well of the gel sequentially, and marker was added to one of the wells. The electrode chamber was placed on the electrophoresis tank, with the power on, the positive and negative poles aligned, and the voltage set to 150 V. After running for 5 min, the voltage was adjusted to 200 V. Electrophoresis was run again for about 35 min and then turn off the power to stop. A Bio-rad gel imager was used to scan and take pictures of the non-stained SDS-PAGE gels, and the pictures were analyzed using Image Lab5.2.1 to calculate protein purity.

2). CE-SDS: The sample was diluted to 2.5 mg/ml with ultrapure water and added to 55 μL Sample Buffer, followed by 2 μL 10 kDa IS and 5 μL 250 mM IAM, and mixed well. The samples were next heated at 70±2° C. for 10±2 min, then cooled to room temperature and transferred to the accompanying sample tube, labeled and ready for sampling.

3). SEC-HPLC: The samples were centrifuged at 15° C. with 10000 rpm for 5 min. The supernatant was collected and analyzed. The relevant SEC chromatographic parameters are listed below:
Mobile phase A: 200 mM PBS pH 6.9;
Mobile phase B: ultrapure water;
Flow rate: 0.7 ml/min;
Wavelength: 280 nm;
Column temperature: room temperature;
Sample analysis time: 20 min;
Injection size: 20 μg.

### 2.2.2 Analysis Results

The expression product purity of the anti-CD20×CD3 FORMAT New7 bispecific antibody was about 84.1% using SDS-PAGE analysis (FIG. 8).

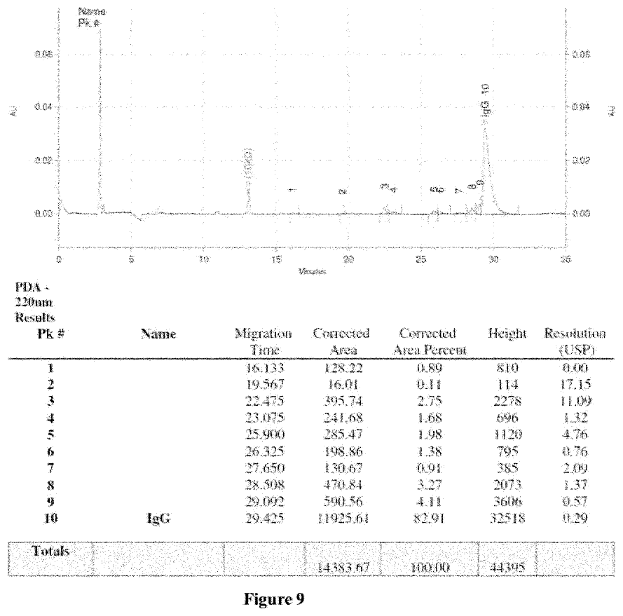

The expression product purity of the anti-CD20×CD3 FORMAT New7 bispecific antibody was about 82.91% using CE-SDS analysis (see, peak #10 in FIG. 9).

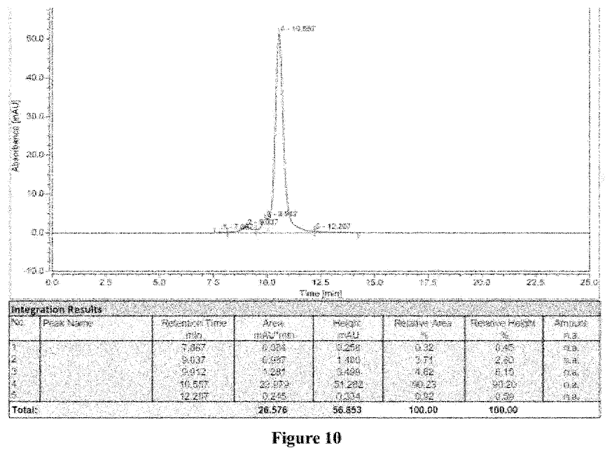

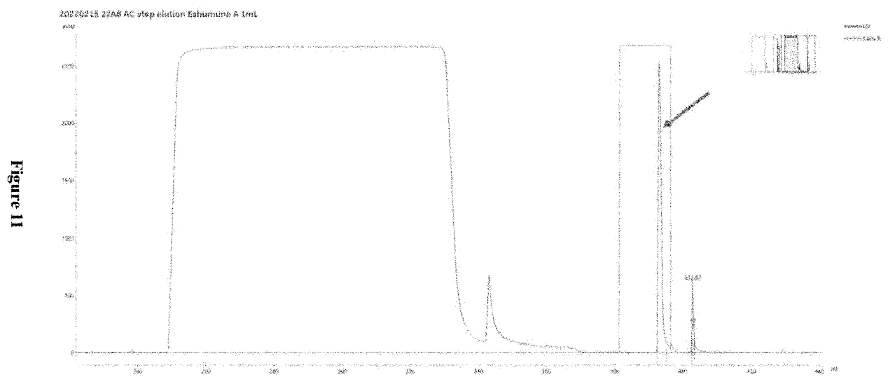

The purity of the expressed anti-CD20×CD3 FORMAT New7 bispecific antibody was about 90.23% using SEC-HPLC analysis (see, peak No 4 in FIG. 10).

In addition to the target protein, there are still a few Low Molecular Weight (LMW) fragments (see, FIGS. 8, 9, and 10) and aggregates of High Molecular Weight (HMW, see FIG. 10) in the expression products. The related products can be distinguished more clearly by SDS-PAGE, CE-SDS and SEC-HPLC. The results are shown in FIGS. 8-10.

### 2.3 Antibody Purification

### 2.3.1 Experimental METHODS

1). Affinity chromatography (AC): The supernatant samples containing the anti-CD20×CD3 FORMAT New7 bispecific antibody obtained from the cell culture were eluted by affinity chromatography at pH 4.0, and the elution peaks were subsequently analyzed.

Chromatography column: 1 ml Mab Select Sure LX (GE) pre-assembled column; equilibration buffer A: 50 mM acetic acid-sodium acetate, pH 6.0; elution buffer B: 50 mM acetic acid-sodium acetate, pH 3.6; neutralization buffer C: 1M Tris-HCl, pH 8.5; flow rate: 1 ml/min; gradient: 0-100% B linear gradient elution. After separation, the eluate was collected at a volume of 1 ml/tube, and 0.1 ml of neutralization buffer C was added to each 1 ml fraction, pH about 6-7.

2). Cation exchange chromatography (CEX): The resulting elutes from previous step were combined and the pH is adjusted to 6.1. Then the combined elutes were filtered and used as the sample CEX-Load. The chromatographic column: Capto S Impact was applied with the following buffers:
EQ/wash/Elution Buffer A: 50 mM NaAc-HAc, pH 6.0
Elution Buffer B: 50 mM NaAc-HAc+1M NaCl, pH6.0
Perform linear gradient elution: gradient: 0-50%1B, 60CV.

### 2.3.2 Purification Results:

### 1). Affinity Chromatography (AC):

The purification profile is shown in FIG. 11. As shown in FIG. 11, the target product was successfully eluted from the affinity chromatography column by the eluent buffer, and a single high peak was detected using UV signal (see the arrow in FIG. 11), indicating that the purified product has high purity.

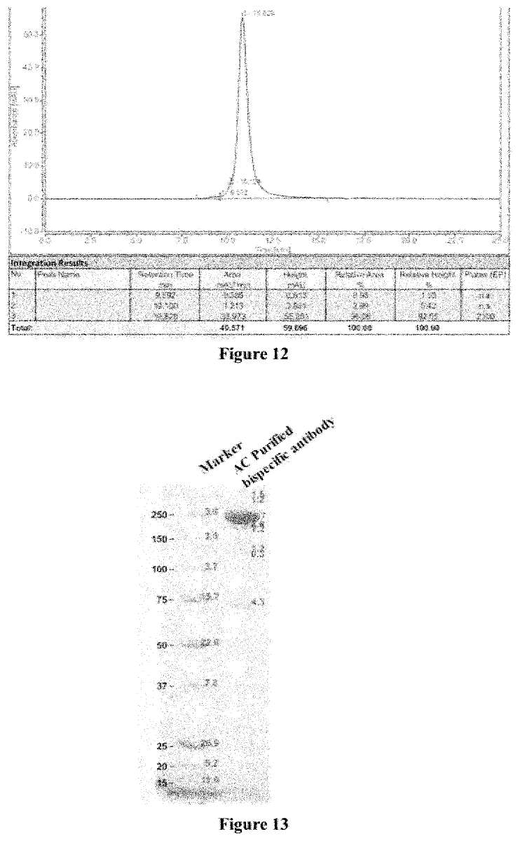

The elute was analyzed by SEC-HPLC for purity, and the result shows the purity was 92.55% (see, peak #3 of FIG. 12), which indicates that AC reduced the LMW content.

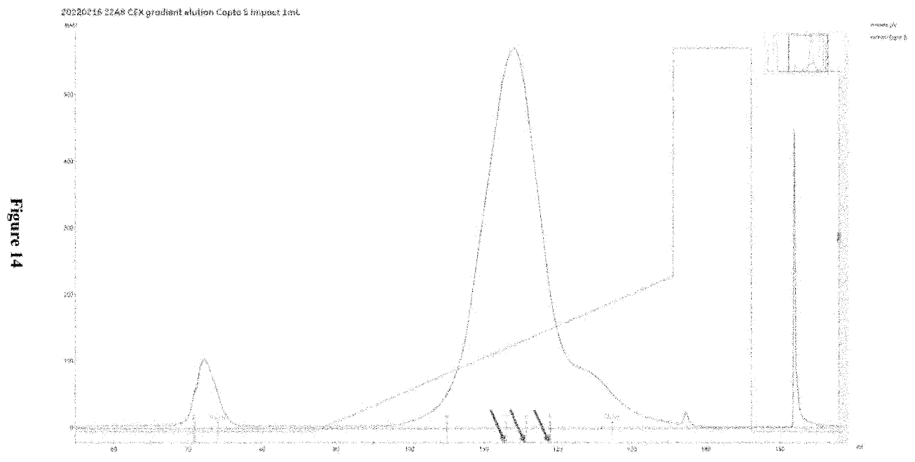

The elute was also analyzed by SDS-PAGE for purity, and the result shows the purity was 85.7% (see FIG. 13), which indicates that there were still LMW after purification in this step (FIG. 13).

Compared with the results observed with the unpurified samples in Example 1, SEC-HPLC and SDS-PAGE results clearly reflected the improvement of purification by the AC process, which also revealed that the impurities were LMW and thus provided a direction for subsequent purification processes.

### 2). Cation Exchange Chromatography (CEX):

To further remove the LMW in the AC purified sample, the elute from the AC process was collected and further purified using CEX. The elution profile after CEX purification is shown in FIG. 14. A major single peak was detected by UV signal (see the peak spanning from the 100 ml to the 120 ml elute), which indicates that target product was successfully eluted by the eluent buffer by linear gradient elution.

Three elute samples (namely C01-C03, see the three arrows in FIG. 14) were collected, and analyzed by SDS-PAGE and SEC respectively for purity.

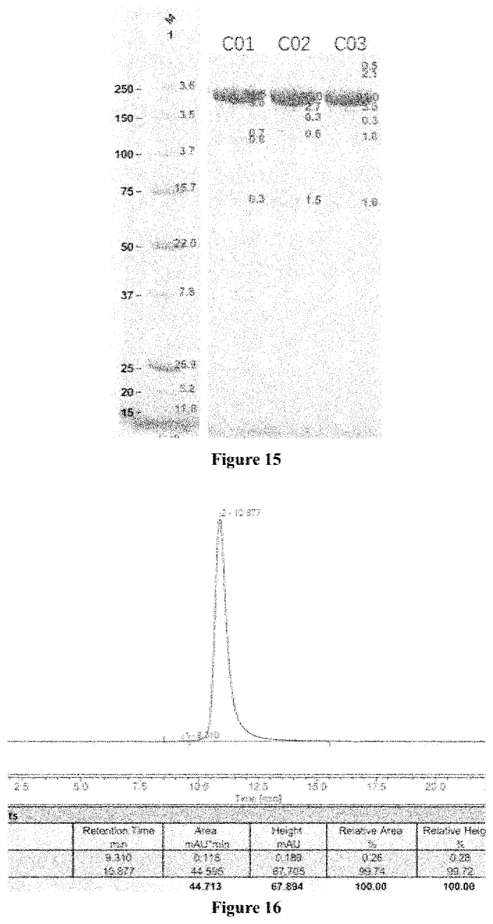

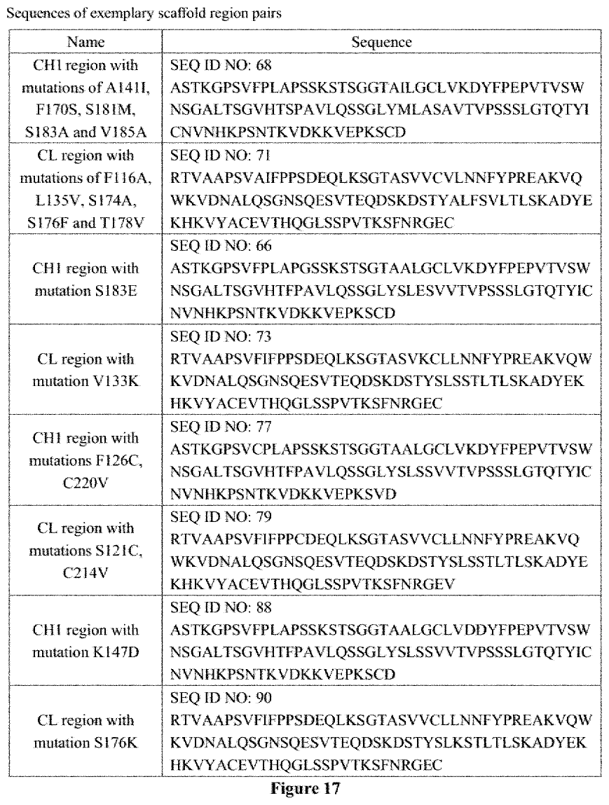

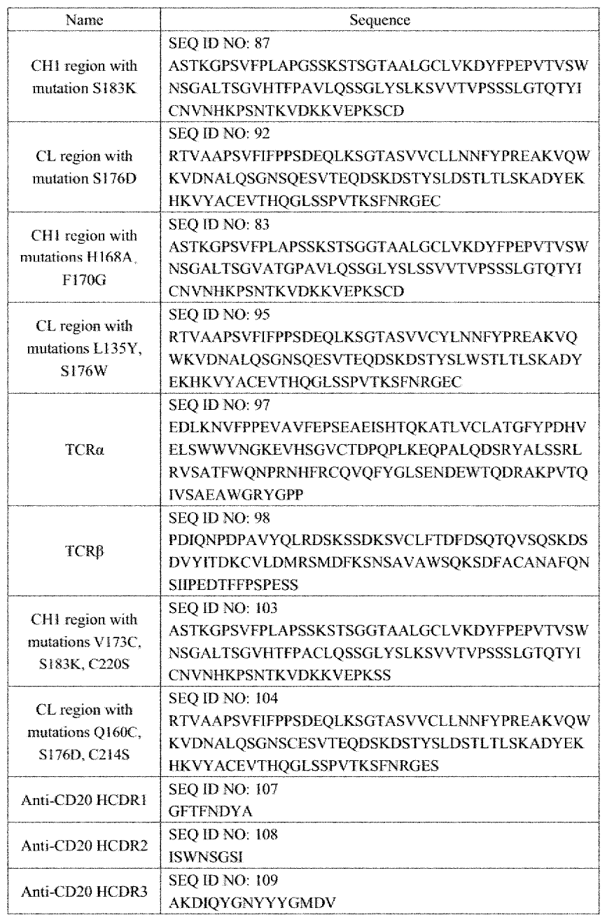

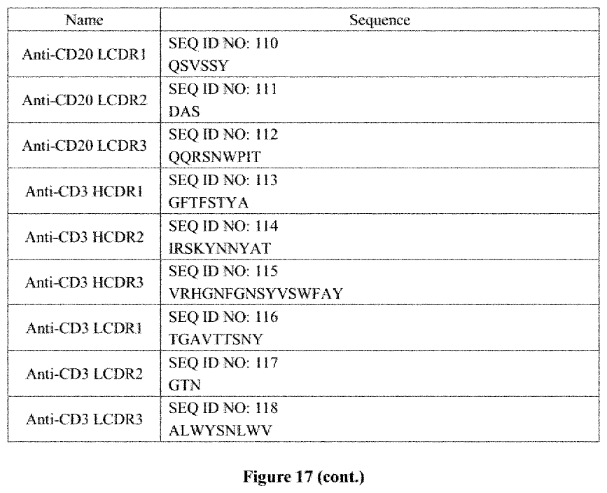

SDS-PAGE showed that the purity results of C01, C02 and C03 were 94.6%, 95.0%, and 91.0%, respectively (FIG. 15). SEC-HPLC showed that the purity results of C01, C02 and C03 were 93.89%, 99.74%, and 97.73%, respectively (see below Table 2). The SEC-HPLC diagram for C02 is shown in FIG. 16.

TABLE 2
Purity analysis of the fractions eluted from CEX by SEC-HPLC

| Samples | Monomer % | Polymer % | Segment % |
|---|---|---|---|
| CEX | 96.06 | 3.94 | / |
| Load | | | |
| C01 | 93.89 | 0.11 | / |
| C02 | 99.74 | 0.26 | / |
| C03 | 97.73 | 2.27 | / |

Notably, after purification by CEX, the purity of the C02 fraction exceeded 99% by SEC-HPLC (FIG. 16) and 95% by SDS-PAGE (FIG. 15), showing a significant improvement in the purity of the CEX-load sample compared with that before CEX purification, which removed most of the LMW and HMW from the AC purified product.

SEC-HPLC and SDS-PAGE clearly reflected the enhancement in purity by the CEX, which also indicated a high degree of homogeneity of the purified final product.

The above results showed that, the novel 2:1 bispecific antibody structure of this invention (FORMAT NEW) can be readily purified using conventional purification methods such as AC and CEX. A purification process as simple as two steps can provide a high purity sample with a purity of over 95%, which indicated that the significant advantage of FORMAT NEW antibodies over the FORMAT EX antibodies in antibody purification, and also suggests the ease of use of the FORMAT NEW antibody structure of the present invention.

Although the examples used anti-CD20×CD3 bispecific antibodies to demonstrate the convenience of purification and high purity, the inventors have also tested other bispecific antibodies constructed with the new bispecific antibody format provided in the present disclosure, and obtained similar results in convenience of purification and high purity. Therefore, it should be understood that the new format provided in the present disclosure is applicable on various kinds of targets and can provide for convenience of purification and high purity for any bispecific antibodies.

What is claimed is:

1. A fusion polypeptide comprising from C terminus to N terminus:
a) a first target-binding fragment A1;
b) a polypeptide linker;
c) a second target-binding fragment B2;
wherein:
the polypeptide linker has a length that is sufficiently short to minimize potential intramolecular interaction between A1 and B2,
the A1 is capable of pairing with a first pairing fragment B1, to form a first target binding domain;
the B2 is capable of pairing with a second pairing fragment A2, to form a second target binding domain; and
the A1 is configured to exhibit less binding to the B2 relative to the B1, and the B2 is configured to exhibit less binding to the A1 relative to the A2.

2. A polypeptide complex, comprising:
a) the fusion polypeptide of claim 1,
b) a second polypeptide comprising the first pairing fragment B1;
c) a third polypeptide comprising the second target binding fragment B2; and
d) a fourth polypeptide and the fifth polypeptide each comprising the second pairing fragment A2;
wherein:
the A1 in the fusion polypeptide pairs with the B1 in the second polypeptide to form a first target binding domain,
the B2 in the fusion polypeptide pairs with the A2 in the fourth polypeptide to form a second target binding domain, and
the A2 in the fifth polypeptide pairs with the B2 in the third polypeptide to form another second target binding domain.

3. The fusion polypeptide of claim 1 or the polypeptide complex of claim 2, wherein at least one of the pair of B1 and A1 and the pair of B2 and A2 contains at least one configuration capable of discouraging mispairing between B1 and A2 and/or between B2 and A1.

4. The fusion polypeptide of claim 1 or 3, or the polypeptide complex of claim 2 or 3, wherein:
the A1 comprises a first antibody variable region VA1 selected from VH1 or VL1,
the B1 comprises a first pairing antibody variable region VB1 capable of pairing with VA1 to form the first target-binding domain, wherein the VB1 is selected from VH1 or VL1, and
the B2 comprises a second antibody variable region VB2 selected from VH2 or VL2, and
the A2 comprises a second pairing antibody variable region VA2 capable of pairing with VB2 to form the second target-binding domain, wherein the VA2 is selected from VH2 or VL2.

5. The fusion polypeptide of claim 4 or the polypeptide complex of claim 4, wherein the VB1 comprises VH1, the VA1 comprises VL1, the VB2 comprises VH2, and the VA2 comprises VL2.

6. The fusion polypeptide of claim 4 or the polypeptide complex of claim 4, wherein:
a) the VB1 comprises VH1, the VA1 comprises VL1, the VB2 comprises VL2, and the VA2 comprises VH2; or
b) the VB1 comprises VL1, the VA1 comprises VH1, the VB2 comprises VH2, and the VA2 comprises VL2.

7. The fusion polypeptide of any one of claims 1, and 3-6 or the polypeptide complex of any one of claims 2-6, wherein the A1 further comprises a first scaffold region SRₐ operably linked to the VA1, and the B2 further comprises a second scaffold region SR_{b} operably linked to the VB2, wherein the SRₐ and the SR_{b} are configured such that pairing between the SRₐ and the SR_{b} is discouraged.

8. The fusion polypeptide of claim 7 or the polypeptide complex of claim 7, wherein the B1 further comprises a first pairing scaffold region PSRₐ that is operably linked to the VB1 and is capable of binding to the SRₐ, and wherein the A2 further comprises a second pairing scaffold region PSR_{b} that is operably linked to the VA2 and is capable of binding to the SR_{b}, optionally, the SRₐ and the PSRₐ are paired by a first disulfide bond, and/or the SR_{b} and the PSR_{b} are paired by a second disulfide bond.

9. The fusion polypeptide of claim 7 or 8, or the polypeptide complex of claim 7 or 8, wherein the pair of SRₐ/PSRₐ or the pair of SR_{b}/PSR_{b} is/are selected from the group consisting of:
a) a pair of heavy chain constant region 1 (CH1) and light chain constant region (CL);
b) a pair of T cell receptor (TCR) constant region alpha (Calpha) and TCR constant region beta (Cbeta);
c) a pair of TCR constant region gamma (Cgamma) and TCR constant region delta (Cdelta);
d) a pair of ligand-binding domain of a receptor and the ligand;
e) a pair of PRD (proline rich domain) and SH3 domain; and
f) a pair of obscurin and titin.

10. The fusion polypeptide of any one of claims 7-9 or the polypeptide complex of any one of claims 7-9, wherein the pair of the SRₐ and the PSRₐ is distinct from the pair of the SR_{b} and the PSR_{b}.

11. The fusion polypeptide of any one of claims 7-10 or the polypeptide complex of any one of claims 7-10, wherein:
a) the pair of the SRₐ and the PSRₐ is a pair of CH1 and CL, and the pair of the SR_{b} and the PSR_{b} is a pair of:
i) Calpha and Cbeta,
ii) Cgamma and Cdelta,
iii) ligand-binding domain of a receptor and the ligand;
iv) PRD (proline rich domain) and SH3 domain, or
v) obscurin and titin,
or
b) the pair of the SR_{b} and the PSR_{b} is a pair of CH1 and CL, and the pair of the SRₐ and the PSRₐ is a pair of:
i) Calpha and Cbeta,
ii) Cgamma and Cdelta,
iii) ligand-binding domain of a receptor and the ligand;
iv) PRD (proline rich domain) and SH3 domain, or
v) obscurin and titin.

12. The fusion polypeptide of any one of claims 7-9 or the polypeptide complex of any one of claims 7-9, wherein both the pair of SRₐ/PSRₐ and the pair of SR_{b}/PSR_{b} are the same, and the pair of SRₐ/PSRₐ and/or the pair of SR_{b}/PSR_{b} are configured such that mispairing between SRₐ/PSR_{b} or between SR_{b}/PSRₐ is discouraged.

13. The fusion polypeptide of claim 12 or the polypeptide complex of claim 12, wherein the pair of SRₐ/PSRₐ comprises a CH1 domain CH1a and a CL domain CLa, and the pair of SR_{b}/PSR_{b} comprises a CH1 domain CH1b and a CL domain CLb.

14. The fusion polypeptide of claim 13 or the polypeptide complex of claim 13, wherein the VB1 comprises VH1, the VA1 comprises VL1, the VB2 comprises VH2, and the VA2 comprises VL2, and wherein:
a) the PSRₐ is CL domain CLa, the SRₐ is CH1 domain CH1a, the SR_{b} is CH1 domain CH1b, the PSR_{b} is CL domain CLb; or
b) the PSRₐ is CH1 domain CH1a, the SRₐ is CL domain CLa, the SR_{b} is CL domain CLb, the PSR_{b} is CH1 domain CH1b.

15. The fusion polypeptide of claim 13 or the polypeptide complex of claim 13, wherein the PSRₐ is CH1 domain CH1a, the SRₐ is CL domain CLa, the SR_{b} is CH1 domain CH1b, the PSR_{b} is CL domain CLb, and wherein:
a) the VB1 comprises VH1, the VA1 comprises VL1, the VB2 comprises VL2, and the VA2 comprises VH2; or
b) the VB1 comprises VL1, the VA1 comprises VH1, the VB2 comprises VH2, and the VA2 comprises VL2.

16. The fusion polypeptide of claim 13 or the polypeptide complex of claim 13, wherein: the VB1 comprises VH1, the VA1 comprises VL1, the VB2 comprises VH2, and the VA2 comprises VL2, the PSRₐ is CH1 domain CH1a, the SRₐ is CL domain CLa, the SR_{b} is CH1 domain CH1b, the PSR_{b} is CL domain CLb, and wherein:
a) the fusion polypeptide comprises an amino acid sequence of Formula (I): VH2-CH1b-Linker-VL1-CLa,
b) the second polypeptide comprises an amino acid sequence of Formula (II): VH1-CH1a,
c) the third polypeptide comprises an amino acid sequence of Formula (III): VH2-CH1b,
d) the fourth and fifth polypeptides each comprising an amino acid sequence of Formula (IV): VL2-CLb;
wherein the pair of CH1b/CLb and/or the pair of CH1a/CLa are configured such that mispairing between CH1b and CLa and/or between CH1a and CLb is discouraged.

17. The fusion polypeptide of claim 16 or the polypeptide complex of claim 16, wherein at least one of the pair of CH1b/CLb and the pair of CH1a/CLa have one or more characteristics shown below:
1) having at least one non-natural disulfide bond that discourages mispairing between CH1b and CLa and/or between CH1a and CLb;
2) having one or more introduced amino acid mutations that form at least one or more introduced charged amino acid residues that discourages mispairing between CH1b and CLa and/or between CH1a and CLb; or
3) having one or more introduced amino acid mutations that form an orthogonal CH1-CL interface that discourages mispairing between CH1b and CLa or between CH1a and CLb.

18. The fusion polypeptide of claim 17 or the polypeptide complex of claim 17, wherein a first CH1/CL pair and a second CH1/CL pair are selected from CH1b/CLb and CH1a/CLa, and wherein the first CH1/CL pair is associated by a first disulfide bond that is non-natural, and optionally lacks a natural disulfide bond or has a disrupted natural disulfide bond.

19. The fusion polypeptide of claim 18 or the polypeptide complex of claim 18, wherein the second CH1/CL pair is associated by a second disulfide bond which is formed at a position different from the first disulfide bond, and optionally the second disulfide bond is a natural disulfide bond.

20. The fusion polypeptide of claim 18 or 19 or the polypeptide complex of claim 18 or 19, wherein the first disulfide bond is formed by two cysteine residues introduced at a set of heavy chain-light chain EU positions selected from the group consisting of:
a) heavy chain EU position 126—light chain EU position 121,
b) heavy chain EU position 173—light chain EU position 160, and
c) heavy chain EU position 128—light chain EU position 118.

21. The fusion polypeptide of any one of claims 18-20, or the polypeptide complex of any one of claims 18-20, wherein the natural disulfide bond is between heavy chain EU position 220 and light chain EU position 214.

22. The fusion polypeptide of any one of claims 18-21, or the polypeptide complex of any one of claims 18-21, wherein the first CH1/CL pair comprises a CH1 comprising substitution at EU position 126 for a cysteine residue, and substitution at EU position 220 for a non-cysteine residue; and a CL comprising substitution at EU position 121 for a cysteine residue, and substitution at EU position 214 for a non-cysteine residue.

23. The fusion polypeptide of any one of the claims 17-22, or the polypeptide complex of any one of the claims 17-22, wherein a first CH1/CL pair and a second CH1/CL pair are selected from CH1b/CLb and CH1a/CLa, and wherein the first CH1/CL pair contains at least one substitution of a non-charged residue to a charged residue, and/or at least one substitution of a charged residue to an oppositely charged residue, such that the first CH1/CL pair contains a first pair of oppositely charged residues that favors pairing of the first CH1/CL pair.

24. The fusion polypeptide of claim 23, or the polypeptide complex of claim 23, wherein the second CH1/CL pair contains at least one substitution of a non-charged residue to a charged residue, and/or at least one substitution of a charged residue to an oppositely charged residue, such that the second CH1/CL pair contains a second pair of oppositely charged residues that favors pairing of the second CH1/CL pair, and optionally the first pair of oppositely charged residues and the second pair of oppositely charged residues discourages pairing of CH1a with CLb or CH1b and CLa.

25. The fusion polypeptide of claim 24, or the polypeptide complex of claim 24, wherein the first pair of oppositely charged residues and/or the second pair of oppositely charged residues are configured such that CH1a and CLb have both positive or both negative charged residues, and/or CH1b and CLa have both positive or both negative charged residues.

26. The fusion polypeptide of any one of claims 23-25, or the polypeptide complex of any one of claims 23-25, wherein the first pair of oppositely charged residues and/or the second pair of oppositely charged residues is introduced at a set of heavy chain-light chain EU positions selected from the group consisting of:
a) heavy chain EU position 183: light chain EU position 176,
b) heavy chain EU position 183: light chain EU position 133,
c) heavy chain EU position 147: light chain EU position 176,
d) heavy chain EU position 141: light chain EU position 116,
e) heavy chain EU position 126: light chain EU position 121, and
f) heavy chain EU position 218: light chain EU position 122.

27. The fusion polypeptide of any one of claims 22-26, or the polypeptide complex of any one of claims 22-26, wherein the pair of oppositely charged residues comprises a positive-charged amino acid residue and a negative-charged amino acid residue, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

28. The fusion polypeptide of any one of claims 17-27, or the polypeptide complex of any one of claims 17-27, wherein a first CH1/CL pair and a second CH1/CL pair are selected from CH1b/CLb and CH1a/CLa, and wherein the first CH1/CL pair comprises one or more introduced amino acid mutations that form an orthogonal CH1-CL interface.

29. The fusion polypeptide of claim 28, or the polypeptide complex of claim 28, wherein the orthogonal CH1-CL interface introduced at a set of heavy chain-light chain EU positions including heavy chain EU positions H168A, F170G, light chain EU positions L135Y, S176W.

30. The fusion polypeptide of any one of claims 17-29, or the polypeptide complex of any one of claims 17-29, wherein the first CH1/CL pair comprises one or more introduced amino acid mutations that form orthogonal Fab designs at a set of heavy chain-light chain EU positions selected from the group consisting of:
a) substitutions at heavy chain EU positions A141I, F170S, S181M, S183A, and V185A, and substitutions at light chain EU positions F116A, A235V, S174A, S176F, and T178V.

31. The fusion polypeptide of any one of claims 16-30 or the polypeptide complex of any one of claims 16-30, wherein a first VH/VL pair and a second VH/VL pair are selected from VH1/VL1 and VH2/VL2, and wherein the first VH/VL pair has at least one substitution of a non-charged residue to a charged residue, and/or at least one substitution of a charged residue to an oppositely charged residue, such that the first VH/VL contains a third pair of oppositely charged residues that favors pairing of first VH/VL pair.

32. The fusion polypeptide of claim 31 or the polypeptide complex of claim 31, wherein the second VH/VL pair has at least one substitution of a non-charged residue to a charged residue, and/or at least one substitution of a charged residue to an oppositely charged residue, such that the second VH/VL contains a fourth pair of oppositely charged residues that favors pairing of second VH/VL pair, and optionally the third pair of oppositely charged residues and the fourth pair of oppositely charged residues discourages pairing of VH1 with VL2 or VH2 with VL1.

33. The fusion polypeptide of claim 32 or the polypeptide complex of claim 32, wherein the third pair of oppositely charged residues and the fourth pair of oppositely charged residues are configured such that VH1 and VL2 have both positive or both negative charged residues, and/or VH2 and VL1 have both positive or both negative charged residues.

34. The fusion polypeptide of any one of claims 31-33 or the polypeptide complex of any one of claims 31-33, wherein the third pair of oppositely charged residues and/or the fourth pair of oppositely charged residues is introduced at a set of heavy chain-light chain EU positions selected from the group consisting of:
a) heavy chain EU position 39: light chain EU position 38;
b) heavy chain EU position 105: light chain EU position 43, and
c) heavy chain EU position 62: light chain EU position 1, or
any combination thereof.

35. The fusion polypeptide of any one of claims 31-34 or the polypeptide complex of any one of claims 31-34, wherein the pair of oppositely charged residues comprises a positive-charged amino acid residue and a negative-charged amino acid residue, wherein the positive-charged amino acid residue is selected from the group consisting of lysine (K), histidine (H) and arginine (R), and/or the negative-charged amino acid residue is selected from the group consisting of aspartic acid (D) and glutamic acid (E).

36. The polypeptide complex of any one of the preceding claims, wherein the second polypeptide and the third polypeptide further comprise an operably linked first dimerization domain and an operably linked second dimerization domain, respectively, that are associated to form a dimer; optionally, the first dimerization domain comprises a first Fc region, and/or the second dimerization domain comprises a second Fc region.

37. The polypeptide complex of claim 36, wherein the first Fc region and/or the second Fc region is derived from IgG1, IgG2, IgG3 or IgG4.

38. The polypeptide complex of claim 36, wherein the first Fc region and the second Fc region have different amino acid sequences and have at least one configuration that facilitates heterodimerization of the first Fc region and the second Fc region.

39. The polypeptide complex of claim 38, wherein the first Fc region comprises a first Fc mutation, and/or the second Fc region comprises a second Fc mutation, wherein:
a) the first Fc mutations comprises T366W or S354C, and the second Fc mutation comprises Y349C, T366S, L368A, or Y407V;
b) the first Fe mutation comprises D399K or E356K, and the second Fc mutation comprises K392D, or K409D;
c) the first Fe mutation comprises E356K, E357K, or D399K, and the second Fe mutation comprises K370E, K409D, or K439E;
d) the first Fe mutation comprises S364H, or F405A, and the second Fc mutation comprises Y349T, or T394F;
e) the first Fe mutation comprises S364H, or T394F, and the second Fe mutation comprises Y394T, or F405A;
f) the first Fe mutation comprises K370D, or K409D, and the second Fe mutation comprises E357K, or D399K; or
g) the first Fe mutation comprises L351D, or L368E, and the second Fc mutation comprises L351K, or T366K,
wherein numbering is according to the EU index.

40. The fusion polypeptide of any one of the preceding claims or the polypeptide complex of any one of the preceding claims, wherein the first target binding domain and the second target binding domain bind to different targets.

41. The fusion polypeptide of any one of the preceding claims or the polypeptide complex of any one of the preceding claims, wherein at least one of the first target binding domain and the second target binding domain is chimeric, humanized, or fully human.

42. The fusion polypeptide of any one of the preceding claims or the polypeptide complex of any one of the preceding claims, wherein at least one of the first target binding domain and the second target binding domain binds to a disease-associated antigen or an immune related target, optionally, the disease-associated antigen is a tumor-associated antigen, an antigen associated with an autoimmune diseases or an inflammatory disease, or an antigen associated with an eye disorder, an antigen associated with central nervous system disease, an antigen associated with an infectious disease or an antigen associated with a coagulation disease.

43. The fusion polypeptide of any one of the preceding claims or the polypeptide complex of any one of the preceding claims, wherein one of the first target binding domain and the second target binding domain binds to a tumor-associated antigen, and the other binds to an immune related target.

44. A nucleic acid comprising a nucleotide sequence encoding the fusion polypeptide of any one of the preceding claims or the polypeptide complex of any one of the preceding claims.

45. A vector comprising the nucleic acid of claim 44.

46. A host cell comprising the nucleic acid of claim 44 or the vector of claim 45.

47. A pharmaceutical composition comprising the polypeptide complex of any one of claims 1-43 and a pharmaceutically acceptable carrier.

48. A conjugate comprising the polypeptide complex of any one of claims 1-43 and a payload conjugated thereto, wherein the payload is selected from the group consisting of a radioactive label, a fluorescent label, an enzyme-substrate label, an affinity purification tag, a tracer molecule, an anticancer drug, and a cytotoxic molecule.

49. A composition comprising the polypeptide complex of any one of claims 1-43, or the conjugate of claim 48, and a pharmaceutically acceptable carrier.

50. A method of treating or preventing from a disease, condition, or symptom comprising administering to a subject in need thereof a therapeutically effective amount of the polypeptide complex of any one of claims 1-43, the pharmaceutical composition of claim 47, the conjugate of claim 48, or the composition of claim 49.

51. The method of claim 50, wherein the disease is selected from the group consisting of a cancer, an inflammatory disease, an infectious or parasitic disease, a cardiovascular disease, eye disease, central nerves system (CNS) disease, an injury, metabolic disease, autoimmune disease or a coagulation disorder.

52. A method of detecting presence or level of an antigen, comprising contacting a sample suspected of containing the antigen with the polypeptide complex of any one of claims 1-43, and determining the formation of a complex between the antigen and the polypeptide complex.


SEQUENCE LISTING (118 sequences)

SEQ ID NO: 1 - protein, 691 aa, synthetic construct
  REGION 1..691: note = synthetic
EVQLVESGGGLVQPGRSLRLSCAASGFTFNDYAMHWVRKAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLESVVTVPSSSLGTQTYICNVNHKPSNTKVDKKVEPKSCDGGGGSGGGGSEVQLVESGGGLVQPGGSLRLSCAASGFTFSTYAMNWVREAPGKGLEWVGRIRSKYNNYATYYADSVKGRFTISRDDSKNSLYLQMNSLRAEDTAVYYCVRHGNFGNSYVSWFAYWGQGTLVTVSSASTKGPSVFPLAPSSKSTSGGTAILGCLVKDYFPEPVTVSWNSGALTSGVHTSPAVLQSSGLYMLASAVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, synthetic construct
  REGION 1..216: note = synthetic
QAVVTQEPSLTVSPGGTVTLTCRSSTGAVTTSNYANWVQKKPGQAPRGLIGGTNKRAPWTPARFSGSLLGGKAALTLSGAQAEDEADYYCALWYSNLWVFGGGTKLTVLRTVAAPSVAIFPPSDEQLKSGTASVVCVLNNFYPREAKVQWKVDNALQSGNSQESVTEQDSKDSTYALFSVLTLSKADYEKHKVYACEVTHQGLSSPVTKSFNRGEC

SEQ ID NO: 3 - protein, 452 aa, synthetic construct
  REGION 1..452: note = synthetic
EVQLVESGGGLVQPGRSLRLSCAASGFTFNDYAMHWVRKAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLESVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 4 - protein, 214 aa, synthetic construct
  REGION 1..214: note = synthetic
EIVLTQSPATLSLSPGERATLSCRASQSVSSYLAWYQEKPGQAPRLLIYDASNRATGIPARFSGSGSGTDFTLTISSLEPEDFAVYYCQQRSNWPITFGQGTRLEIKRTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 455 aa, synthetic construct
  REGION 1..455: note = synthetic
EVQLVESGGGLVQPGGSLRLSCAASGFTFSTYAMNWVREAPGKGLEWVGRIRSKYNNYATYYADSVKGRFTISRDDSKNSLYLQMNSLRAEDTAVYYCVRHGNFGNSYVSWFAYWGQGTLVTVSSASTKGPSVFPLAPSSKSTSGGTAILGCLVKDYFPEPVTVSWNSGALTSGVHTSPAVLQSSGLYMLASAVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 452 aa, synthetic construct
  REGION 1..452: note = synthetic
EVQLVESGGGLVQPGRSLRLSCAASGFTFNDYAMHWVRKAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLESVVTVPSSSLGTQTYICNVNHKPSNTKVDKKVEPKSCDGGGGSGGGGSQAVVTQEPSLTVSPGGTVTLTCRSSTGAVTTSNYANWVQKKPGQAPRGLIGGTNKRAPWTPARFSGSLLGGKAALTLSGAQAEDEADYYCALWYSNLWVFGGGTKLTVLRTVAAPSVAIFPPSDEQLKSGTASVVCVLNNFYPREAKVQWKVDNALQSGNSQESVTEQDSKDSTYALFSVLTLSKADYEKHKVYACEVTHQGLSSPVTKSFNRGEC

SEQ ID NO: 7 - protein, 452 aa, synthetic construct; identical to SEQ ID NO: 3
  REGION 1..452: note = synthetic

SEQ ID NO: 8 - protein, 214 aa, synthetic construct; identical to SEQ ID NO: 4
  REGION 1..214: note = synthetic

SEQ ID NO: 9 - protein, 691 aa, synthetic construct
  REGION 1..691: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, synthetic construct
  REGION 1..216: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 11 - protein, 452 aa, synthetic construct
  REGION 1..452: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 12 - protein, 214 aa, synthetic construct
  REGION 1..214: note = synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 455 aa, synthetic construct
  REGION 1..455: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 452 aa, synthetic construct
  REGION 1..452: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 15 - protein, 452 aa, synthetic construct; identical to SEQ ID NO: 11
  REGION 1..452: note = synthetic

SEQ ID NO: 16 - protein, 214 aa, synthetic construct; identical to SEQ ID NO: 12
  REGION 1..214: note = synthetic

SEQ ID NO: 17 - protein, 691 aa, synthetic construct
  REGION 1..691: note = synthetic
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGDGTTVTVSSASTKGPSVFPLAPGSSKSTSGTAALGCLVKDYFPEPVTVSWNSGALTSGVHTFPAVLQSSGLYSLKSVVTVPSSSLGTQTYICNVNHKPSNTKVDKKVEPKSCDGGGGSGGGGSEVQLVESGGGLVQPGGSLRLSCAASGFTFSTYAMNWVRKAPGKGLEWVGRIRSKYNNYATYYADSVKGRFTISRDDSKNSLYLQMNSLRAEDTAVYYCVRHGNFGNSYVSWFAYWGKGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 216 aa, synthetic construct
  REGION 1..216: note = synthetic
QAVVTQEPSLTVSPGGTVTLTCRSSTGAVTTSNYANWVQDKPGQDPRGLIGGTNKRAPWTPARFSGSLLGGKAALTLSGAQAEDEADYYCALWYSNLWVFGGGTKLTVLRTVAAPSVFIFPPSDEQLKSGTASVVCLLNNFYPREAKVQWKVDNALQSGNSQESVTEQDSKDSTYSLKSTLTLSKADYEKHKVYACEVTHQGLSSPVTKSFNRGEC

SEQ ID NO: 19 - protein, 452 aa, synthetic construct
  REGION 1..452: note = synthetic
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGDGTTVTVSSASTKGPSVFPLAPGSSKSTSGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 20 - protein, 214 aa, synthetic construct
  REGION 1..214: note = synthetic
EIVLTQSPATLSLSPGERATLSCRASQSVSSYLAWYQKKPGQKPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLDSTLTLSKADYEKHKVYACEVTHQGLSSPVTKSFNRGEC

SEQ ID NO: 21 - protein, 455 aa, synthetic construct
  REGION 1..455: note = synthetic
EVQLVESGGGLVQPGGSLRLSCAASGFTFSTYAMNWVRKAPGKGLEWVGRIRSKYNNYATYYADSVKGRFTISRDDSKNSLYLQMNSLRAEDTAVYYCVRHGNFGNSYVSWFAYWGKGTLVTVSSASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 452 aa, synthetic construct
  REGION 1..452: note = synthetic
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGDGTTVTVSSASTKGPSVFPLAPGSSKSTSGTAALGCLVKDYFPEPVTVSWNSGALTSGVHTFPAVLQSSGLYSLKSVVTVPSSSLGTQTYICNVNHKPSNTKVDKKVEPKSCDGGGGSGGGGSQAVVTQEPSLTVSPGGTVTLTCRSSTGAVTTSNYANWVQDKPGQDPRGLIGGTNKRAPWTPARFSGSLLGGKAALTLSGAQAEDEADYYCALWYSNLWVFGGGTKLTVLRTVAAPSVFIFPPSDEQLKSGTASVVCLLNNFYPREAKVQWKVDNALQSGNSQESVTEQDSKDSTYSLKSTLTLSKADYEKHKVYACEVTHQGLSSPVTKSFNRGEC

SEQ ID NO: 23 - protein, 452 aa, synthetic construct; identical to SEQ ID NO: 19
  REGION 1..452: note = synthetic

SEQ ID NO: 24 - protein, 214 aa, synthetic construct; identical to SEQ ID NO: 20
  REGION 1..214: note = synthetic

SEQ ID NO: 25 - protein, 691 aa, synthetic construct
  REGION 1..691: note = synthetic
EVQLVESGGGLVQPGRSLRLSCAASGFTFNDYAMHWVRYAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFTFSTYAMNWVRKAPGKGLEWVGRIRSKYNNYATYYADEVKGRFTISRDDSKNSLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 216 aa, synthetic construct
  REGION 1..216: note = synthetic
RAVVTQEPSLTVSPGGTVTLTCRSSTGAVTTSNYANWVQDKPGQAPRGLIGGTNKRAPWTPARFSGSLLGGKAALTLSGAQAEDEADYYCALWYSNLWVFGGGTKLTVLRTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 27 - protein, 452 aa, synthetic construct
  REGION 1..452: note = synthetic
EVQLVESGGGLVQPGRSLRLSCAASGFTFNDYAMHWVRYAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 28 - protein, 214 aa, synthetic construct
  REGION 1..214: note = synthetic
EIVLTQSPATLSLSPGERATLSCRASQSVSSYLAWYQRKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 455 aa, synthetic construct
  REGION 1..455: note = synthetic
EVQLVESGGGLVQPGGSLRLSCAASGFTFSTYAMNWVRKAPGKGLEWVGRIRSKYNNYATYYADEVKGRFTISRDDSKNSLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 452 aa, synthetic construct
  REGION 1..452: note = synthetic
EVQLVESGGGLVQPGRSLRLSCAASGFTFNDYAMHWVRYAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSRAVVTQEPSLTVSPGGTVTLTCRSSTGAVTTSNYANWVQDKPGQAPRGLIGGTNKRAPWTPARFSGSLLGGKAALTLSGAQAEDEADYYCALWYSNLWVFGGGTKLTVLRTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 31 - protein, 452 aa, synthetic construct; identical to SEQ ID NO: 27
  REGION 1..452: note = synthetic

SEQ ID NO: 32 - protein, 214 aa, synthetic construct; identical to SEQ ID NO: 28
  REGION 1..214: note = synthetic

SEQ ID NO: 33 - protein, 715 aa, synthetic construct
  REGION 1..715: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 204 aa, synthetic construct
  REGION 1..204: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 35 - protein, 452 aa, synthetic construct; identical to SEQ ID NO: 11
  REGION 1..452: note = synthetic

SEQ ID NO: 36 - protein, 214 aa, synthetic construct; identical to SEQ ID NO: 12
  REGION 1..214: note = synthetic

SEQ ID NO: 37 - protein, 479 aa, synthetic construct
  REGION 1..479: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 440 aa, synthetic construct
  REGION 1..440: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 39 - protein, 452 aa, synthetic construct; identical to SEQ ID NO: 11
  REGION 1..452: note = synthetic

SEQ ID NO: 40 - protein, 214 aa, synthetic construct; identical to SEQ ID NO: 12
  REGION 1..214: note = synthetic

SEQ ID NO: 41 - protein, 677 aa, synthetic construct
  REGION 1..677: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 232 aa, synthetic construct
  REGION 1..232: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 452 aa, synthetic construct
  REGION 1..452: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 44 - protein, 214 aa, synthetic construct
  REGION 1..214: note = synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 441 aa, synthetic construct
  REGION 1..441: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 468 aa, synthetic construct
  REGION 1..468: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 452 aa, synthetic construct; identical to SEQ ID NO: 43
  REGION 1..452: note = synthetic

SEQ ID NO: 48 - protein, 214 aa, synthetic construct; identical to SEQ ID NO: 44
  REGION 1..214: note = synthetic

SEQ ID NO: 49 - protein, 690 aa, synthetic construct
  REGION 1..690: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLKSVVTVPSSSLGTQTYICNVNHKPSNTKVDKKVEPKSSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 216 aa, synthetic construct
  REGION 1..216: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLRTVAAPSVFIFPPSDEQLKSGTASVVCLLNNFYPREAKVQWKVDNALQSGNSCESVTEQDSKDSTYSLDSTLTLSKADYEKHKVYACEVTHQGLSSPVTKSFNRGES

SEQ ID NO: 51 - protein, 452 aa, synthetic construct; identical to SEQ ID NO: 11
  REGION 1..452: note = synthetic

SEQ ID NO: 52 - protein, 214 aa, synthetic construct; identical to SEQ ID NO: 12
  REGION 1..214: note = synthetic

SEQ ID NO: 53 - protein, 454 aa, synthetic construct
  REGION 1..454: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPACLQSSGLYSLKSVVTVPSSSLGTQTYICNVNHKPSNTKVDKKVEPKSSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 452 aa, synthetic construct
  REGION 1..452: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLRTVAAPSVFIFPPSDEQLKSGTASVVCLLNNFYPREAKVQWKVDNALQSGNSCESVTEQDSKDSTYSLDSTLTLSKADYEKHKVYACEVTHQGLSSPVTKSFNRGES

SEQ ID NO: 55 - protein, 452 aa, synthetic construct; identical to SEQ ID NO: 11
  REGION 1..452: note = synthetic

SEQ ID NO: 56 - protein, 214 aa, synthetic construct; identical to SEQ ID NO: 12
  REGION 1..214: note = synthetic

SEQ ID NO: 57 - protein, 695 aa, synthetic construct
  REGION 1..695: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
  REGION 1..214: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 452 aa, synthetic construct; identical to SEQ ID NO: 11
  REGION 1..452: note = synthetic

SEQ ID NO: 60 - protein, 214 aa, synthetic construct; identical to SEQ ID NO: 12
  REGION 1..214: note = synthetic

SEQ ID NO: 61 - protein, 459 aa, synthetic construct
  REGION 1..459: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 450 aa, synthetic construct
  REGION 1..450: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 63 - protein, 452 aa, synthetic construct; identical to SEQ ID NO: 11
  REGION 1..452: note = synthetic

SEQ ID NO: 64 - protein, 214 aa, synthetic construct; identical to SEQ ID NO: 12
  REGION 1..214: note = synthetic

SEQ ID NO: 65 - protein, 122 aa, synthetic construct
  REGION 1..122: note = synthetic
EVQLVESGGGLVQPGRSLRLSCAASGFTFNDYAMHWVRKAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 66 - protein, 104 aa, synthetic construct
  REGION 1..104: note = synthetic
ASTKGPSVFPLAPGSSKSTSGTAALGCLVKDYFPEPVTVSWNSGALTSGVHTFPAVLQSSGLYSLESVVTVPSSSLGTQTYICNVNHKPSNTKVDKKVEPKSCD

SEQ ID NO: 67 - protein, 125 aa, synthetic construct
  REGION 1..125: note = synthetic
EVQLVESGGGLVQPGGSLRLSCAASGFTFSTYAMNWVREAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 68 - protein, 104 aa, synthetic construct
  REGION 1..104: note = synthetic
ASTKGPSVFPLAPSSKSTSGGTAILGCLVKDYFPEPVTVSWNSGALTSGVHTSPAVLQSSGLYMLASAVTVPSSSLGTQTYICNVNHKPSNTKVDKKVEPKSCD

SEQ ID NO: 69 - protein, 226 aa, synthetic construct
  REGION 1..226: note = synthetic
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 109 aa, synthetic construct
  REGION 1..109: note = synthetic
QAVVTQEPSLTVSPGGTVTLTCRSSTGAVTTSNYANWVQKKPGQAPRGLIGGTNKRAPWTPARFSGSLLGGKAALTLSGAQAEDEADYYCALWYSNLWVFGGGTKLTVL

SEQ ID NO: 71 - protein, 107 aa, synthetic construct
  REGION 1..107: note = synthetic
RTVAAPSVAIFPPSDEQLKSGTASVVCVLNNFYPREAKVQWKVDNALQSGNSQESVTEQDSKDSTYALFSVLTLSKADYEKHKVYACEVTHQGLSSPVTKSFNRGEC

SEQ ID NO: 72 - protein, 107 aa, synthetic construct
  REGION 1..107: note = synthetic
EIVLTQSPATLSLSPGERATLSCRASQSVSSYLAWYQEKPGQAPRLLIYDASNRATGIPARFSGSGSGTDFTLTISSLEPEDFAVYYCQQRSNWPITFGQGTRLEIK

SEQ ID NO: 73 - protein, 107 aa, synthetic construct
  REGION 1..107: note = synthetic
RTVAAPSVFIFPPSDEQLKSGTASVKCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 122 aa, synthetic construct
  REGION 1..122: note = synthetic
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 75 - protein, 104 aa, synthetic construct
  REGION 1..104: note = synthetic
ASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 76 - protein, 125 aa, synthetic construct
  REGION 1..125: note = synthetic
EVQLVESGGGLVQPGGSLRLSCAASGFTFSTYAMNWVRQAPGKGLEWVGRIRSKYNNYATYYADSVKGRFTISRDDSKNSLYLQMNSLRAEDTAVYYCVRHGNFGNSYVSWFAYWGQGTLVTVSS

SEQ ID NO: 77 - protein, 104 aa, synthetic construct
  REGION 1..104: note = synthetic
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSVD

SEQ ID NO: 78 - protein, 109 aa, synthetic construct
  REGION 1..109: note = synthetic
QAVVTQEPSLTVSPGGTVTLTCRSSTGAVTTSNYANWVQQKPGQAPRGLIGGTNKRAPWTPARFSGSLLGGKAALTLSGAQAEDEADYYCALWYSNLWVFGGGTKLTVL

SEQ ID NO: 79 - protein, 107 aa, synthetic construct
  REGION 1..107: note = synthetic
RTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEV

SEQ ID NO: 80 - protein, 107 aa, synthetic construct
  REGION 1..107: note = synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 81 - protein, 107 aa, synthetic construct [sequence omitted]

SEQ ID NO: 82 - protein, 122 aa, synthetic construct
  REGION 1..122: note = synthetic
EVQLVESGGGLVQPGRSLRLSCAASGFTFNDYAMHWVRYAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 83 - protein, 104 aa, synthetic construct
  REGION 1..104: note = synthetic
ASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCD

SEQ ID NO: 84 - protein, 226 aa, synthetic construct
  REGION 1..226: note = synthetic
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 85 - protein, 125 aa, synthetic construct
  REGION 1..125: note = synthetic
EVQLVESGGGLVQPGGSLRLSCAASGFTFSTYAMNWVRKAPGKGLEWVGRIRSKYNNYATYYADSVKGRFTISRDDSKNSLYLQMNSLRAEDTAVYYCVRHGNFGNSYVSWFAYWGKGTLVTVSS

SEQ ID NO: 86 - protein, 122 aa, synthetic construct
  REGION 1..122: note = synthetic
EVQLVESGGGLVQPGRSLRLSCAASGFTFNDYAMHWVRDAPGKGLEWVSTISWNSGSIGYADSVKGRFTISRDNAKKSLYLQMNSLRAEDTALYYCAKDIQYGNYYYGMDVWGDGTTVTVSS

SEQ ID NO: 87 - protein, 104 aa, synthetic construct
  REGION 1..104: note = synthetic
ASTKGPSVFPLAPGSSKSTSGTAALGCLVKDYFPEPVTVSWNSGALTSGVHTFPAVLQSSGLYSLKSVVTVPSSSLGTQTYICNVNHKPSNTKVDKKVEPKSCD

SEQ ID NO: 88 - protein, 104 aa, synthetic construct
  REGION 1..104: note = synthetic
ASTKGPSVFPLAPSSKSTSGGTAALGCLVDDYFPEPVTVSWNSGALTSGVHTFPAVLQSSGLYSLSSVVTVPSSSLGTQTYICNVNHKPSNTKVDKKVEPKSCD

SEQ ID NO: 89 - protein, 109 aa, synthetic construct
  REGION 1..109: note = synthetic
QAVVTQEPSLTVSPGGTVTLTCRSSTGAVTTSNYANWVQDKPGQDPRGLIGGTNKRAPWTPARFSGSLLGGKAALTLSGAQAEDEADYYCALWYSNLWVFGGGTKLTVL

SEQ ID NO: 90 - protein, 107 aa, synthetic construct
  REGION 1..107: note = synthetic
RTVAAPSVFIFPPSDEQLKSGTASVVCLLNNFYPREAKVQWKVDNALQSGNSQESVTEQDSKDSTYSLKSTLTLSKADYEKHKVYACEVTHQGLSSPVTKSFNRGEC

SEQ ID NO: 91 - protein, 107 aa, synthetic construct
  REGION 1..107: note = synthetic
EIVLTQSPATLSLSPGERATLSCRASQSVSSYLAWYQKKPGQKPRLLIYDASNRATGIPARFSGSGSGTDFTLTISSLEPEDFAVYYCQQRSNWPITFGQGTRLEIK

SEQ ID NO: 92 - protein, 107 aa, synthetic construct
  REGION 1..107: note = synthetic
RTVAAPSVFIFPPSDEQLKSGTASVVCLLNNFYPREAKVQWKVDNALQSGNSQESVTEQDSKDSTYSLDSTLTLSKADYEKHKVYACEVTHQGLSSPVTKSFNRGEC

SEQ ID NO: 93 - protein, 125 aa, synthetic construct
  REGION 1..125: note = synthetic
EVQLVESGGGLVQPGGSLRLSCAASGFTFSTYAMNWVRKAPGKGLEWVGRIRSKYNNYATYYADEVKGRFTISRDDSKNSLYLQMNSLRAEDTAVYYCVRHGNFGNSYVSWFAYWGQGTLVTVSS

SEQ ID NO: 94 - protein, 109 aa, synthetic construct
  REGION 1..109: note = synthetic
RAVVTQEPSLTVSPGGTVTLTCRSSTGAVTTSNYANWVQDKPGQAPRGLIGGTNKRAPWTPARFSGSLLGGKAALTLSGAQAEDEADYYCALWYSNLWVFGGGTKLTVL

SEQ ID NO: 95 - protein, 107 aa, synthetic construct
  REGION 1..107: note = synthetic
RTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 96 - protein, 107 aa, synthetic construct
  REGION 1..107: note = synthetic
EIVLTQSPATLSLSPGERATLSCRASQSVSSYLAWYQRKPGQAPRLLIYDASNRATGIPARFSGSGSGTDFTLTISSLEPEDFAVYYCQQRSNWPITFGQGTRLEIK

SEQ ID NO: 97 - protein, 132 aa, synthetic construct
  REGION 1..132: note = synthetic
EDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRYGPP

SEQ ID NO: 98 - protein, 95 aa, synthetic construct
  REGION 1..95: note = synthetic
PDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 99 - protein, 104 aa, synthetic construct
  REGION 1..104: note = synthetic
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 100 - protein, 104 aa, synthetic construct
  REGION 1..104: note = synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 101 - protein, 107 aa, synthetic construct
  REGION 1..107: note = synthetic
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 107 aa, synthetic construct
  REGION 1..107: note = synthetic
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 103 aa, synthetic construct
  REGION 1..103: note = synthetic
ASTKGPSVFPLAPSSKSTSGGTAALGCLVKDYFPEPVTVSWNSGALTSGVHTFPACLQSSGLYSLKSVVTVPSSSLGTQTYICNVNHKPSNTKVDKKVEPKSS

SEQ ID NO: 104 - protein, 107 aa, synthetic construct
  REGION 1..107: note = synthetic
RTVAAPSVFIFPPSDEQLKSGTASVVCLLNNFYPREAKVQWKVDNALQSGNSCESVTEQDSKDSTYSLDSTLTLSKADYEKHKVYACEVTHQGLSSPVTKSFNRGES

SEQ ID NO: 105 - protein, 103 aa, synthetic construct
  REGION 1..103: note = synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 106 - protein, 108 aa, synthetic construct
  REGION 1..108: note = synthetic
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECD

SEQ ID NO: 107 - protein, 8 aa, synthetic construct
GFTFNDYA

SEQ ID NO: 108 - protein, 8 aa, synthetic construct
ISWNSGSI

SEQ ID NO: 109 - protein, 15 aa, synthetic construct
AKDIQYGNYYYGMDV

SEQ ID NO: 110 - protein, 6 aa, synthetic construct
QSVSSY

SEQ ID NO: 111 - skipped

SEQ ID NO: 112 - protein, 9 aa, synthetic construct
QQRSNWPIT

SEQ ID NO: 113 - protein, 8 aa, synthetic construct
GFTFSTYA

SEQ ID NO: 114 - protein, 10 aa, synthetic construct
IRSKYNNYAT

SEQ ID NO: 115 - protein, 16 aa, synthetic construct
VRHGNFGNSYVSWFAY

SEQ ID NO: 116 - protein, 9 aa, synthetic construct
TGAVTTSNY

SEQ ID NO: 117 - skipped

SEQ ID NO: 118 - protein, 9 aa, synthetic construct
ALWYSNLWV